SEQUENCE LISTING (30 sequences)

SEQ ID NO: 1 - protein, 20 aa, Streptomyces thermoautotrophicus
  misc_feature (18)..(18): Xaa can be any naturally occurring amino acid
ALPQTELRPMGKPILRKXDP

SEQ ID NO: 2 - protein, 30 aa, Streptomyces thermoautotrophicus
MFPNAFKYEAPASVDEAVRLLAEYGYDGKV

SEQ ID NO: 3 - protein, 18 aa, Streptomyces thermoautotrophicus
MKIRVKVNGTLYEADVEP

SEQ ID NO: 4 - protein, 33 aa, Streptomyces thermoautotrophicus
MFELPPLPYPYDALEPYFDAKKMEIHYYGGHGA

SEQ ID NO: 5 - DNA, 2493 bp, Streptomyces thermoautotrophicus
gtggcactgccgcagactgaactgcgcccgatgggcaaaccgattctccgcaaggaggatccccggctgatccgcgggaagggccggtttgtggacgacatcctgttgccgaatatgctccatctttgcatcttgcggagcccgtacgcccacgcccgcattcgccgcatcgatacgtcgaaagcagaagccgcgccgggcgtcaagctggtgctcacgggagaagatctggccaagatgaacctcgcctggatgccgaccttggcgggggacgtgcagatggtgctggcgacgggcaaggtcctgttccagtaccaggaggtcgcggcggtcgtcgcggagacgcgcgcccaggccgaggacgcgattcagctgatcgaggtcgactacgagcccctgccggtggtggtcgatccgttcaaggcgctggagccggacgcgcccatcctccgggaggacaaggagaaaaagtcgaaccacatctggcactgggaggcgggcgaccgggaagagaccgacgcgatcttccgcgaagcgccggtcgtcgtcaagcaggatgtgcgttttcagcgcgtccatccctcgccgcttgaaccgtgcggctgcgtggccgactacaacccggcgacggggaagctcgtggtctacgtcacgtcgcaggcgccgcacgtccaccggacggcgatcgctttgacgacgggcttccccgaacacatgattcaggtcatttcgcccgatgtgggcggcgggttcgggaacaaggtgcccctctaccccggctacgtggtggcgatcgtcgcttccttgaagctgggagtccccgtgaagtggatcgagacgcggacggaaaacatcgccagcacccacttcgcccgcgattaccacatgacggcggagatcgcggcgacggaagacggcaagatgctggcgctccgcgtgaagacgatcgccgaccacggcgcgttcgacgcgaccgccaacccgaccaaataccccgccggattgtacagcatcgtgacggggtcgtacgacttcaaggcggcgttcgtcgaagtggacggtgttcatacgaacaaaccgccgggcggtgtggcctaccgctgctcgttccgggtcacggaagcctcctatctgattgaacgcgtcgtggacgttttggcccgtcggctcaagatggatccggccgagttgcgcctgcgcaatttcattcgcaaggagcagttcccgtaccgcagcccgacgggatgggtgtacgacagcggggattacgaaaagacgttcaagctcgcgctggagcgcatcggatatgaagagctgcgcaaggagcagaaggagaagtgggcccggggagaattcatgggcatcggcatctccaccttcacggagatcgtcggcgcgggtccggcgcactccttcgatattctcggcatcaagatgttcgacagcgcggagatccgcgtccatccgacgggcaaggtgatcgcccggctcggcgtgcgccatcagggacaggggcatgagacgacgttcgcccagatcatcgccgaggagctggggctcagcgtcgacgacgtcgtggtcgaagaaggcgataccgacacggccccctacgggttgggcacgtacgccagccgttccacgccgacggccggggcggcggcggccctctgtgcgcgccggatccgggacaaggcgcgtaagatcgcggcccatttgctggaggtcaacgaagacgacgtcgtctgggacggcgccgccttttcggtcaagggacttccgggccgttcggtgacgatgaaagatgtggcctttgccgcctacacgaacgtgcccgacggcatcgagccgggcttggaggcgtcgtactactacaatccgccgaacctcaccttcccctacggggcctacatcgccgtggtcgacatcgacaagggaacgggcgccgtgaaggtgcggcggttcttggccgtcgacgattgcggcaacgtgatcaatccgatgatcgtcgaaggtcaggtgcacggcggcctgacggaaggatttgcgatcgcgttcatgcaggacatcccgtatgacgccgacggcaactgcctggcgccgaactggatggactacctggttcccaccgcttgggacacgccccagctggagacggatcggacggtcacgccctcgcctcaccatccgcttggcgccaaaggggtcggcgagtcgcccaacgtcggttcgccggcggcgttcgtcaatgcggtgctggacgcgctgtcgccgctcggcgtagaacacatcgacatgccgatctatccgtggaaggtgtggaagatcttgcgggacacggcattacggagtgattcgatggccattcctgcgtcattccagagcgcgaggagggaaaagcccggaggcggtatagcctccgggcccatcaaatggacaacctctgggagacagcgagggcgttggatgaacgcgcggagccttacgtctgggtga

SEQ ID NO: 6 - DNA, 516 bp, Streptomyces thermoautotrophicus
atgaagatccgggtcaaagtcaacgggacgctgtacgaggcggacgtggaaccgcggacgcttctggcgtactttctgcgcgaggaattgaagttgacgggcacgcacatcggctgcgacacgaccacctgcggagcttgcacggtgcttttggacgggaaggcggtcaagtcgtgcacggtcctcgcggtgcaggcgaacggacgcgaggtcatgacggtcgaagggctggaaaaagacggccagctgcatcctctgcaagtcgcgttctgggaagaacacgcgcttcattgcggatattgcacgcccggtatgttgatggcctcttacgcgctgttgcaagaaaatccgatgcccaccgaggaagagattcgttttggattgtccgggaacgtctgccgttgcaccggttacatgaacatcgtcaaggccgttcaatccgcggcgcgcaggctttccggcgcgtccggcgaagccgttggggaggtggcgaccagtggcactgccgcagactga

SEQ ID NO: 7 - DNA, 885 bp, Streptomyces thermoautotrophicus
gtgtttcccaatgcgttcaagtacgaggcgccggcatcggtcgacgaggccgtccgtctgctggccgagtacggctacgacggaaaggtgttggcgggcgggcagagcttgctcccgatgatgaagctgcgcgtcgcggcgccggccgtgctcatcgacatcaacggcatcgatgcgctccaggggtggcgcgaggtcgacgggaaactgcgggtgggcgcgatgacgcgccacgccgaactggagcatgccaaagagctccgcgacacgtatccgctgtttttccagacggcccgatggatcgccgatccgctcatccgcaaccgcgggaccatcggaggctcgctcgcgcacgccgatcccggctccgactggggggcggcgatgatcgcgcttcgggccgaagtggaagcgcgaggcccccagggaagccggctcattcccatcgacgaattttttgtcgatacgtttgcaaccgctttaaatgaagacgaactcgccgtcgcggtgcacgtgccgacgccgaaggggccggcggcctcccggtatatgaagctggagcgccgggcgggcgatttcgccatcgccgcgctcgccgtccacgtcgccctcggaaccgacggccgcgtgtccgaagccggcatcggcatttgcgcgtgcggtccgatccccctccgggcagccaaagcggaggcggcgctcatcggccggccgctgacggaagaggtcatcgtcgaggcgtcgaggctggttccggaagatgccgagcccgccgacgatctgcgaggaagcgcggaatataagcgcgacgtgttgcgcgtgtttgccgcgcgcgccctccgcgacatcgccaaagagctgcaaggaaaggtggggatccaatga

SEQ ID NO: 8 - DNA, 624 bp, Streptomyces thermoautotrophicus
atgttcgaactgccgccgcttccgtacccctacgacgcgctggagccgtattttgacgccaagacgatggaaattcactacaacgggcaccacggcgcttacgtcaagaacctgaacgccgccctcgaaaaatatcccgcatggcaaaataagccgattgaagagctgcttcagtccctcgaccaactgccggaagacatccggacggcggtccggaacaacggcgggggccactacaaccacagcttctggtggccgatgctgaagaaaaacgaagggggccagccggtcggcaagtttgccgaagcgatcaaccgggacttcggcagctttgaggcctttaaggacgccttttccaaggcggcggcgggacggttcggaagcggctgggcgtgggtcgtcgtcgaaccggatgggaagctcaccgtcacgacgacgccgaaccaggacaacccggtcatggaagggaagacggtcgtcttcggcctcgacgtctgggagcacgcctactacctgaagtatcagaaccggcggccggagtacatccaggcgttctggaacgtcgtcaactgggacgtcgtcaacgagcggtacgaagaagcgctgaaaaagttcgggcggtaa

SEQ ID NO: 9 - DNA, 24206 bp, Klebsiella pneumoniae
ggtaacccgctacggcttgagattatccgcatccttgccgacggcagcgagcagagctgtaacgccctgcgtcacgaagatgtggcgaagtcgaccatgacccaccactggcgcgtcctgcgcgacagcggtgtgatctggcagcgcccacaggggcgggagaacttgatttcgctgcgccgggaagatttagacgcgcgctttcccggcctgctggatacgctgcttaaggtcatgcagcaggagaactaaaggcccgctactcctcgccggccagccgccgatactgggcaaagcgggcccgcgcgtcctcctcggttcggctaaagagcgcatccgccagatgcggcgtcgttttgtgcagcgaggcgtagcgcacttcgccaagcaaaaagtcgcggaagctctcctccggctcttcggaatcgagcataaacggcgtcttaccttccgcttcccgctgcggatgatagcgccacaggtgccagtatcccgcctcaaccgcccgtttcgcctcgcgctggctgcagcgcataccggctttcagcccgtggttaatgcaggcggcgtaggcaatcaccagcgacggtcccggccaggcttcggcctcggcgatcgcccgtagggtctgatctttatcagcgcccatcgcgacctgggccacgtacacattgccgtagctcatcgccatcatgccgagatcttttttccgcgtgcgtttgccctgcgcggcaaacttcgcgatggccgccaccggggtcgatttagacgactggccgccggtattggagtaaacctcggtgtcaaacaccagaatattgacgtcttccccgctcgccagcacgtgatcgagaccgccgaagccgatatcgtaggcccagccgtcgccgccgaaaatccactgcgaacgacgaacaaaatagtcgcggttctgccacagctgctccaacagcggcacgccctctttttccgccgccagccgttcgctgagccggtccgcgcgctcgcgggtgccctcgccttcatcctgcttcgccagccactggcgcattgcgtcgctaagttcgtcgctgaccggtagcgccagcgcggcggtcatatcatcggcgatttgttgacgcaccgcctggccgccgagcatcatgccgaggccaaactccgcattatcctcaaacagcgagttcgcccatgccgggccatggccgcggtggttggtggtatagggaatcgacggcgcgctggctccccagatagaagagcagccggtggcgttagcgatcagcatccggtcgccaaacagctgggttatcaggcgggcataaggcgtttcaccgcatcccgcgcaggcgccggaaaactccagcagcggggtttcaaactggctgcctttgaccgtcgtcttacgaaacggattgctcttcggcgtcagcgccagcgcatagtcccagaccggcgccatctgacgctggctatcgagagactgcatttttaacgccttgccgcgcgcgggacagatatccacgcagttgccgcagccggaacaatccagcggcgagatagccagatggtagtgatactccttcgctccctgcgcgggtttgctcagcagcccaaccggcgcggcgtcatgctcttcgccgttgagcagcgccgggcggatcgccgcatgcgggcagataaaggcgcactggttacactgcgtgcagccctccggctgccagaccggcacttccagcgcgatcccgcgtttctcccacgcggcggtgcccgaaggaaaggtcccgtcctccataccgacgaacgcgctcaccggcagctggtcgccgcactggcggttcatcggctgcagaatatcgcggatgaaatccggcatcatggctgatgcttgcgccgcgggttcatccagcgtcgcccagtgcgccggaatcgtcacctgatgcagcgaggccatgcccagctcgatcgcccgctggttcatctcaatcaccgccgcccctttgctgccgtagcttttttcaaccgcctgcttgaggtaatccgccgcggtctgcgggtcgataatcgccgccagcttaaagaacgccgcctgcatcagcatattaaagcgcccgcccagcccgagctcgcgggcgatatccacggcgttcagggtataaaaatggatattttcccgcgccagatagcgtttaaagccgaccggcagatgctgctccagctccgcatcggaccagctgcagttgagtaaaaaggtcccgcccggctttaatccgtccagcagatcgtagcgctcaacgtaggactgctgcgaacaggagataaaatcggcccgatggatcaggtagggcgaattgatcggccggtcgccgaagcgtaaatgtgaaacggtaatgccgccggattttttcgagtcataagaaaagtaggcctgcgcgtagagcggcgttttatcgccgataattttgatcgcgcttttattggccccgacggtgccgtccgagcccatgccccaaaatttacaggcggtgatgccgtcatgcgagaccgccagcgtctgctgggccggcggtaacgaagtaaaggttacatcatcgacaatcccgagggtaaacccgtccatcggcagcggtttattgaggttatcaaagacggccgcgatatcgttgggcagaacatccttcccgccaagcgcatagcggccgccgacgattagcggcgcatcgtcgtggtggtagaaggcgtttttcacatccaggcacagcggttcagcctgagcgccgggctctttggtacggtcaaggacggcaatccgctgcacggttttcggcagctgggcgaagaagtgggccagcgaaaaagggcgaaacagatgcacgctgagcagcccgaccttctctcccgccgcgttcagcgtatccaccacttcctgaacggtatcgcagaccgatcccattgcgataatcacccgttcggcatccgccgcgccggtatagttaaacagatgatactcccggccggtgagcgcgctgatttgcgtcatatagctttcgacaatgtcgggcagcgcctgataaaaacggttgcccgcctcccgctcctggaagtagatatccgggttctgcgccgttccgcggatgaccggatgatccggatgcagcgcgttacggcggaagctgtcgagcgcgggccggtccagcagcgtcgccagctgctcatattccaacacctcgattttttgaatttcgtgcgaggtgcgaaaaccgtcgaagaagttaacaaacgggatgcgtcccttaatcgccgccagatgcgccaccgccgacaaatccatcacctgctgcacgttgttctccgccagcatcgcgcagccggtctggcggaccgccatcacatcctggtgatcgccaaaaatattcagcgaattggtcgccagcgcccgggcgctgacgtgaaagacgcccggcagcagttcaccggcgattttgtacatgttggggatcatcagcagcagcccctgggaggccgtataggtggtggtgagcgccccggcctgcagcgcgccgtggaccgcgcctgccgcgccggcctccgactgcatctccattaagcgcaccggctggccaaaaaggttctttttcccctgcgccgcccactcgtcgacgttttccgccatcggcgtggagggggttatggggtaaatcgccgcgacctcggtaaaggcataagagatccaggccgccgcggcgttgccatccattgttttcatttttccggacattgttcaatcctcgaaggtgagaggcatcttcgccgcctcaaataagcggcaaacccagttgttgcctcaagcacagcctgtgccagctcgcggatgacagaagagttagcgcgaattcaacgcgttatgaagagagtcgccgcgcagcgcgccaagagattgcgtggaataagacacagggggcgacaagctgttgaacaggcgacaaagcgcccatggccccggcaggcgcaattgttctgtttcccacatttggtcgccttattgtgccgttttgttttacgtcctgcgcggcgacaaataactaacttcataaaaatcataagaatacataaacaggcacggctggtatgttccctgcacttctctgctggcaaacactcaacaacaggagaagtcaccatgaccatgcgtcaatgcgctatttacggtaaaggcggtatcggtaaatccaccaccacgcagaacctcgtcgccgcgctggcggagatgggtaagaaagtgatgatcgtcggctgcgatccgaaggcggactccacccgtctgattctgcacgccaaagcacagaacaccattatggagatggccgcggaagtcggctcggtcgaggacctcgaactcgaagacgtgctgcaaattggctacggcgatgtgcgctgcgcggaatccggcggcccggagccaggcgtcggctgcgcgggacgcggcgtgatcacggcgatcaactttcttgaagaagaaggcgcctacgaggacgatctcgatttcgtgttctatgacgtgctcggcgacgtggtctgcggcggcttcgccatgccgatccgcgaaaacaaagcccaggagatctacatcgtctgctccggcgaaatgatggcgatgtacgcggccaacaatatctccaaagggatcgttaaatacgccaaatccggcaaggtgcgcctcggcggcctgatctgtaactcacgtcagaccgaccgtgaagacgaactgattattgccctggcggaaaagctcggtacccagatgatccactttgtgccccgcgacaacatcgtgcagcgcgcggagatccgccgcatgacggttatcgagtacgaccccgcctgtaaacaggccaacgaataccgcaccctggcgcagaagatcgtcaacaacaccatgaaagtggtgccgacgccctgcaccatggatgagctggaatcgctgctgatggagttcggcatcatggaagaggaagacaccagcatcattggcaaaaccgccgccgaagaaaacgcggcctgagcacaggacaattatgatgaccaacgcaacgggcgaacgtaatctggcgctgatccaggaagtcctggaggtgttcccggaaaccgcgcgaaaagagcgcagaaagcacatgatggtcagcgatccgaaaatgaagagcgtcggcaagtgcattatctctaaccgcaaatcacaacccggcgtaatgaccgtacgcggctgcgcctacgccggttccaaaggggtggtatttgggccgattaaggatatggcccatatttcgcacggaccggctggctgcggccagtattcccgcgccgaacgacgcaactactacaccggagtcagcggcgtcgatagcttcggcacgctgaacttcacctctgattttcaggagcgcgacatcgtcttcggcggcgataaaaagctcagcaagctgattgaagagatggagttgctgttcccgctcaccaaagggatcaccattcagtcggaatgcccggtggggctgatcggtgatgatatcagcgcggtggccaacgccagcagcaaggcgctggataaaccggtgatcccggtacgctgcgaaggctttcgcggcgtgtcgcagtctctggggcaccatatcgccaacgacgtggtgcgcgactggatcctgaacaatcgcgaaggacagccgtttgaaaccaccccttacgatgtggcgatcatcggcgactacaacatcggcggcgacgcctgggcctcgcgcattctgctggaagagatggggctacgggtagtcgcgcagtggtccggcgacggcacgctggtggagatggagaataccccattcgtcaagctgaacctggttcactgctaccgttcgatgaactatatcgcccgccatatggaggagaaacatcagattccgtggatggagtacaacttcttcgggccgaccaaaatcgccgaatcgctgcgcaaaatcgccgaccagttcgacgataccattcgcgcgaacgccgaagcggtgatcgcccggtatgaggggcagatggcggcgattatcgccaaatatcgcccgcgcctggaggggcgtaaggtgctgctctatatcggaggcctgcggccgcgccacgttattggcgcctatgaggatctcgggatggagatcatcgccgccggctacgagtttgcccataacgatgattacgaccgcaccctgccggatctgaaagagggcacgctgctgttcgatgacgccagcagctacgagctggaagcgttcgtcaaggcgctgaagcccgaccttatcggctccggcatcaaggaaaaatatatcttccagaaaatgggcgtgccgttccgccagatgcactcgtgggactattccggcccgtaccacggctacgatggtttcgccattttcgcccgcgatatggatatgaccctgaacaacccggcgtggaacgaactgaccgctccgtggctgaagtctgcgtgattgcccactcactgtcccgtctgttcaccgatttgtggcgcgggaggagaacaccatgagccaaacgattgataaaattaatagctgttatccgctattcgaacaggatgaataccaggagctgttccgcaataagcggcagctggaagaggcgcacgatgcgcagcgcgtgcaggaggtctttgcctggaccaccaccgccgagtatgaagcgctgaatttccgacgcgaggcgctgaccgttgacccggcgaaagcctgccagccgcttggcgcggtgctttgctcgctgggatttgccaacaccctgccgtatgtgcacggctctcaggggtgcgtggcctactttcgcacctattttaaccgccatttcaaagagccgatcgcctgcgtctccgactcgatgaccgaagacgcggcggtcttcggcggcaacaacaatatgaacctgggcctgcagaacgccagcgcgctgtacaaaccggagatcattgcggtgtccaccacctgcatggcggaagttatcggcgatgacctgcaggcgtttatcgccaacgctaaaaaagatggcttcgtcgacagcagcatcgccgtgccccacgcccatacgccaagctttatcggcagccacgtcaccggctgggataacatgtttgaaggcttcgccaaaaccttcactgcggactaccaggggcagccgggcaaattgccgaagctcaatctggtgaccggctttgaaacctatctcggcaacttccgcgtattaaagcggatgatggaacagatggcggtgccgtgcagcctgctctccgatccgtcggaagttctcgacacgcccgccgacggtcactatcggatgtattccggcggcaccacgcagcaggagatgaaagaggcccctgacgccatcgatacgctgctcctgcagccgtggcagctgctgaagagcaaaaaagtggtgcaggagatgtggaaccagcccgccaccgaggtcgccattccgctggggctggccgccaccgatgaactgctgatgaccgtcagccagcttagcggcaagccgattgccgacgccctcacccttgagcgcggccggctggttgacatgatgctcgactcccacacctggctgcacggcaagaagtttggcctgtacggcgatccggacttcgtgatgggcctcacccgcttcctgctggagctgggctgcgagccaacggtgatcctgagccataacgccaacaaacgctggcaaaaagcgatgaacaaaatgctcgatgcctcgccgtacgggcgcgatagcgaagtgtttatcaactgcgatttgtggcacttccgttcgctgatgttcacccgtcagccggactttatgatcggcaactcctacggcaagtttatccagcgcgataccctggcgaagggtaaagcctttgaagtgccgcttatccgcctcggctttccgctgttcgaccgccaccatctgcaccgccagacaacctggggttatgaaggggcgatgaacattgtgacgacgctggtgaacgccgtgctggagaaactggatagcgataccagccagctgggcaaaaccgattacagcttcgatctcgtccgttaaccatcaggtgccccgcgtcatgcggcggcaggagggagtatgcccatcgtgattttccgtgagcgcggcgcggacctgtacgcctatatcgcgaaacaggatctggaagcgcgagtgatccagattgagcataacgacgctgaacgctggggcggcgcgatttcgctggaggggggacgccgctactacgtgcatccgcagccggggcgtcccgtctttccgataagcctgcgcgcgacgcgcaataccttgatataaggagctagtgatgtccgacaacgataccctattctggcgtatgctggcgctgtttcagtctctgccggacctacagccggcgcaaatcgtcgactggctggcgcaggagagcggcgagacgctgacgccagagcgtctggcgaccctgacccagccgcagctggccgccagctttccctccgcgacggcggtgatgtcccccgctcgctggtcgcgggtgatggcgagcctgcagggcgcgctgcccgcccatttacgcatcgttcgccctgcccagcgcacgccgcagctgctggcggcattttgctcccaggatgggctggtgattaacggccatttcggccagggacgactgttttttatctacgcgttcgatgaacaaggcggctggttgtacgatctgcgccgctatccctccgccccccaccagcaggaggccaacgaagtgcgcgcccggcttattgaggactgtcagctgctgttttgccaggagataggcgggcccgccgccgcgcggccgatccgccatcgcatccacccgatgaaagcgcagcccgggacgacgattcaggcacagtgcgaggcgatcaatacgctgctggccggccgtttgccgccgtggctggcgaagcggcttaacagggataaccctctggaagaacgcgttttttaatccctgttttgtgcttgttgcccgctgaccccgcgggctttttttcgcgtatggacgctcttccccacgttacgctcaggggaatattccgttcacggttgttccgggcttcttgatgcgcctaaccccctcgctgccagcctttcatcaacaaatagccatcccagcgcgataggtcataaagcatcacatgccgccatcccttgtccgattgttggctttgtcgcaaagccaacaacctcttttctttaaaaatcaaggctccgctctggagcgcgaattgcatcttccccctcatcccccaccgtcaacgaggtcactatgaagggaaatgaaattctggcgctgctggatgaaccggcctgtgaacacaaccataaacaaaaatccggctgcagcgcgcccaaacccggcgccaccgccgcgggctgcgcgttcgacggcgcgcagataaccctgctgcccatcgccgacgtggcgcatctggtccacggccccatcggctgcgccggaagctcatgggataaccgcggcagcgccagctccggccccacccttaatcggctcgggttcaccaccgatctcaacgaacaggacgtgattatgggccgcggcgaacgccgactgtttcacgccgtgcgccatatcgtcacccgctatcatccggcggcggtctttatctacaacacctgcgtaccggccatggagggcgatgacctggaagcggtatgccaggccgcgcagaccgccaccggcgtaccggttatcgctattgacgccgccggtttctacggcagtaaaaatctcggtaaccggccggcgggcgacgtcatggtcaaacgggtcatcggccagcgcgagcccgccccctggccggagagcacgctctttgccccggagcagcgtcacgatattggcctgattggcgaattcaatattgccggcgagttctggcatattcagccgctgctcgacgaactggggatccgcgtgctcggcagcctctccggtgatggccgcttcgccgagatccagaccatgcaccgggcgcaggccaatatgctggtctgctcgcgggcgttaattaacgtcgccagagccctggagcagcgctacggcacgccgtggttcgaaggcagcttttacgggatccgcgccacctctgacgccctgcgccagctggcggcgctgctgggcgacgacgaccttcgccagcgcaccgaagcgctgattgcgcgggaggaacaggcggcggaactggcgctacagccgtggcgcgaacagctgcgcggccgcaaagcgctgctctataccggcggggtgaaatcctggtcggtggtatcggcgctgcaggatttgggcatgaccgtggtggcaaccggcacgcgtaaatccaccgaagaggataaacagcggatccgcgagctgatgggcgaagaggcggtaatgctggaagagggcaacgcccgcacgctgctggatgtggtctatcgctatcaggccgacctgatgattgccggcggacgcaatatgtacaccgcctataaagccaggctgccgtttctcgatatcaatcaggagcgcgaacacgccttcgctggctatcaggggatcgtcaccctcgcccgccagctgtgtcagaccatcaacagccccatctggccgcaaacccattctcgcgccccgtggcgctaaggagctcaccatggcagacattttccgcaccgataagccgctggcggtcagccccatcaaaaccggccagccgctcggcgcaatcctcgccagcctcgggatcgaacacagcatccctctggtccacggcgcgcaggggtgcagcgccttcgccaaagtcttttttattcaacatttccacgacccggttcccctgcagtcgacggcgatggaccccacgtcgacgattatgggcgcggacggcaatatttttaccgccctggataccctctgccagcgcaacaatccgcaggctatcgtactgctcagcaccgggctgtcggaggcccagggcagcgatatttcccgcgtggttcgccagtttcgcgaagagtatccccggcataagggggtggcgatattgacggttaacacgccggatttttatggctccatggagaacggcttcagcgcggtgttagagagcgtcattgagcagtgggtgccgccggcgccgcgcccggctcagcgcaatcgccgggtcaatctgctggtcagccatctctgttcgccgggcgatatcgagtggctgcgccgatgcgtcgaagcctttggtctgcagccgataatcctgccggacctggcgcaatcgatggacggccacctggcgcagggcgatttctcgccgctgacccagggcgggacgccgctgcgccagatagagcagatggggcaaagcctgtgcagcttcgccattggcgtctcccttcatcgcgcctcatcgctgctggccccgcgctgccgcggcgaggttatcgccctgccgcacctgatgaccctcgaacgctgcgacgcctttattcatcaactggcgaaaatttccggacgcgccgttcccgagtggctggaacgccagcgcggccagctacaggatgcgatgatcgactgccatatgtggctccagggccagcgcatggcgatagcggcggaaggcgatttgctggcggcgtggtgtgatttcgccaacagccaggggatgcagcccggcccgctggtggcccctaccggtcatcccagcctgcgccagctgccggtggaacgggtggtgccgggggatctggaggatctgcaaaccctgctgtgcgcgcatcccgccgacctgctggtggcgaactcgcacgcccgcgacctggcggagcagtttgcgctgccgctggtgcgcgcgggttttccgctctttgacaagctcggcgaattccgccgggtgcgacaggggtatagcgggatgcgcgatacgctgtttgagctggcaaacctgatacgcgagcgtcaccaccacctcgcccactaccgatcgccgctgcgccagaaccccgaatcgtcactctccacaggaggcgcttatgccgccgattaaccgtcagtttgatatggtccactccgatgagtggtctatgaaggtcgccttcgccagctccgactatcgtcacgtcgatcagcacttcggcgctaccccgcggctggtggtgtacggcgtcaaggcggatcgggtcactctcatccgggtggttgatttctcggtcgagaacggccaccagacggagaagatcgccaggcggatccacgccctggaggattgcgtcacgctgttctgcgtggcgattggcgacgcggtttttcgccagctgttgcaggtgggcgtgcgtgccgaacgcgttcccgccgacaccaccatcgtcggcttactgcaggagattcagctctactggtacgacaaagggcagcgcaaaaatcagcgccagcgcgacccggagcgctttacccgtctgctgcaggagcaggagtggcatggggatccggacccgcgccgctagccgtgtcgtttctgtgacaaagcccacaaaacatcgcgacactgtaggacgaaccttgtcaggactaatacacaaccatttgaaaaatattaattttattctctggtatcgcaattgctagttcgttatcgccaccgcgcttccgcggtgaaccgcgccccggcgttttccgtcaacatccctggagctgacagcatgtggaattactccgagaaagtgaaagaccatttttttaacccccgcaatgcgcgcgtggtggacaacgccaacgcggtaggcgacgtcggttcgttaagctgcggcgacgccctgcgcctgatgctgcgcgtcgacccgcaaagcgaaatcattgaggaggcgggcttccagaccttcggctgcggcagcgccatcgcctcctcctccgcgctgacggagctgattatcggccataccctcgccgaagccgggcagataaccaatcagcagattgccgattatctcgacggactgccgccggagaaaatgcactgctcggtgatgggccaggaggccctgcgcgcggccatcgccaactttcgcggcgaaagccttgaagaggagcacgacgagggcaagctgatctgcaaatgcttcggcgtcgatgaagggcatattcgccgcgcggtacagaacaacgggctgaccacccttgccgaggtgatcaactacaccaaagcgggcggcggctgcacctcttgccacgaaaaaatcgagctggccctggcggagatcctcgcccagcagccgcagacgacgccagccgtggccagcggcaaagatccgcactggcagagcgtcgtcgataccatcgcagaactgcggccgcatattcaggccgacggcggcgatatggcgctactcagcgtcaccaaccaccaggtgaccgtcagcctctccggcagctgtagcggctgcatgatgaccgatatgaccctggcctggctgcagcaaaaactgatggaacgtaccggctgttatatggaagtggtggcggcctgagccggcgttaactgacccaagggggacaagatgaaacaggtttatctcgataacaacgccaccacccgtctggacccgatggtcctggaagcgatgatgccctttttgaccgatttttacggcaacccctcgtcgatacacgattttggcattccggcccaggcggctctggaacgcgcgcatcagcaggctgcggcgctgctgggcgcggagtatcccagcgagatcatctttacctcctgcgccaccgaagccaccgccaccgccatcgcctcggcgatcgccctgctgcctgagcgtcgcgaaatcatcaccagcgtggtcgaacatccggcgacgctggcggcctgcgagcacatggagcgcgagggctaccggattcatcgcatcgcggtagatggcgagggggcgctggacatggcgcagttccgcgcggcgctcagcccgcgcgtcgcgttggtcagcgtgatgtgggcgaataacgaaaccggggtgcttttcccgatcggcgaaatggcggagctggcccatgaacaaggggcgctgtttcactgcgatgcggtgcaggtggtcgggaaaataccgatcgccgtgggccagacccgcatcgatatgctctcctgctcggcgcataagttccacgggccaaaaggcgtaggctgtctttatctgcggcggggaacgcgctttcgcccgctgctgcgcggcggtcaccaggagtacggtcggcgagccgggacagaaaatatctgcggaatcgtcggcatgggcgcggcctgcgagctggcgaatattcatctgccgggaatgacgcatatcggccaattgcgcaacaggctggagcatcgcctgctggccagcgtgccgtcggtcatggtgatgggcggcggccagccggcggtgcccggcacggtgaatctggcctttgagtttattgaaggtgaagccattctgctgctgttaaaccaggccgggatcgccgcctccagcggcagcgcctgcacctcaggctcgctggaaccctcccacgtgatgcgggcgatgaatatcccctacaccgccgcccacggcaccatccgcttttctctctcgcgctacacccgggagaaagagatcgattacgtcgtcgccacgctgccgccgattatcgaccggctgcgcgcgctgtcgccctactggcagaacggcaagccgcgcccggcggacgccgtattcacgccggtttacggctaaggcggaggtggctgatggaacgcgtgctgattaacgataccaccctgcgcgacggcgagcagagccccggcgtcgcctttcgcaccagcgaaaaggtcgccattgccgaggcgctttacgccgcaggaataacggcgatggaggtcggcaccccggcgatgggcgacgaggagatcgcgcggatccagctggtgcgtcgccagctgcccgacgcgaccctgatgacctggtgtcggatgaacgcgctggagatccgccagagcgccgatctgggcatcgactgggtggatatctcgattccggcttcggataagctgcggcagtacaaactgcgcgagccgctggcggtgctgctggagcggctggcgatgtttatccatcttgcgcataccctcggcctgaaggtatgcatcggctgcgaggacgcctcgcgggccagcggccagaccctgcgcgctatcgccgaggtcgcgcagcaatgcgccgccgcccgcctgcgctatgccgatacggtcggcctgctcgacccttttaccaccgcggcgcaaatctcggccctgcgcgacgtctggtccggcgaaatcgaaatgcatgcccataacgatctgggtatggcgaccgccaatacgctggcggcggtaagcgccggggccaccagcgtgaatacgacggtcctcggtctcggcgagcgggcgggcaacgcggcgctggaaaccgtcgcgctgggccttgaacgctgcctgggcgtggagaccggcgtgcatttttcggcgctgcccgcgtcctgtcagagggtcgcggaagccgcgcagcgcgccatcgacccgcagcagccgctggtcggcgagctggtgtttacccatgagtcaggtgtccacgtggcggcgctgctgcggcacagcgagagctaccagtccatcgccccttccctgatgggccgcagctaccggctggtgctgggcaaacactccgggcgtcaggcggtcaacggcgtttttgaccagatgggctatcacctcaacgccgcgcagattaaccagctgctgcccgccatccgccgcttcgccgagaactggaagcgcagcccgaaagattacgagctggtggctatctacgacgagctgtgcggtgaatccgctctgcgggcgagggggtaatgatggagtggttttatcaaattcccggcgtggacgaacttcgctccgccgaatctttttttcagtttttcgccgtcccctatcagcccgagctgcttggccgctgcagcctgccggtgctggcaacgtttcatcgcaaactccgcgcggaggtgccgctgcaaaaccggctcgaggataacgaccgcgcgccctggctgctggcgcgaagactgctcgcggagagctatcagcaacagtttcaggagagcggaacatgagaccgaaattcacctttagcgaagaggtccgcgtcgtacgcgcgattcgtaacgacggcaccgtggcgggcttcgcgcccggcgcgctgctggtcaggcgcggcagcaccggctttgtgcgcgactggggcgtttttttgcaagatcagattatctaccagatccactttccggaaaccgatcggatcatcggctgccgcgagcaggagctgatccccatcacccagccgtggctggccggaaatttgcaatacagggatagcgtgacctgccagatggcgctcgcggtcaacggcgatgtggtcgtgagcgccggccagcggggacgcgttgaggctaccgatcggggagagctcggcgacagctacaccgtcgactttagcggccgctggttcagggtcccggtgcaggccatcgcccttatagaggaaagagaagaatgaacccgtggcaacgttttgcccggcagcggctggcgcgcagccgctggaatcgcgatccggcggccctggatccggccgacacgccggcttttgaacaggcctggcaacgccagtgccatatggagcagacgatcgtcgcgcgggtccctgaaggcgatattccggcggcgttgctggagaatatcgctgcctcccttgccatctggctcgacgagggggattttgcgccgcccgagcgcgctgccatcgtgcgccatcacgcccggctggaactcgccttcgccgatatcgcccgccaggcgccgcagccggatctctccacggtacaggcatggtatctgcgccaccagacgcagtttatgcgcccggaacagcgtctgacccgccatttactgctgacggtcgataacgaccgcgaagccgtgcaccagcggatcctcggcctgtatcggcaaatcaacgcctcgcgggacgctttcgcgccgctggcccagcgccattcccactgcccgagcgcgctggaagagggtcgtttaggctggattagccgtggcctgctctatccgcagctcgagaccgcgctgttttcactggcggaaaacgcgctaagccttcccatcgccagcgaactgggctggcatcttttatggtgcgaagcgattcgccccgccgcgcccatggagccgcagcaggcgctggagagcgcgcgcgattatctttggcagcagagccagcagcgccatcagcgccagtggctggaacagatgatttcccgtcagccgggactgtgcgggtagcctcggcggctacccgttaacgcctacagcacggtgcgtttaatctcctcaagccagctcgccagacgcgcttcggtctggtcgaactggttatcctgatccagcaccagcccaacaaagcggtcgccttccagcgccgaggacgcgctgaattcataaccctcatttggccagctgccaatcatctgcgcgccgcgcgcgctcagggcgtcgaacagcgggcgcatcccgctgacgaagttgtccggatagcctctctgatcgccgaggccgaacagcgccacggttttccctttcaggctggcgtcgtcgaggccgctgataaattcgctccatgactcgctttcgcatccggcctccagccccggcagctggccgtcgccgagcgtcggcgtgcccagcagcagcaccggataggccataaagtcgtccagcgtcgtgcggttaatgttgaccggggcatccgccagctcgcccagttgcttatggatcattttcgcgattttgcgggttttaccggtatcggtgccaaagaaaataccaatgttcgccatgttgcgctcctgtcggaaaagggggttgaaaatacgcgttctcgcaggggtattgcgaaggctgtgccaggttgctttgcactaccgcggcccatccctgccccaaaacgatcgcttcagccctctcccgccgcgcgcggcggggctggcggggcgcttaaaatgcaaaaagcgcctgcttttcccctaccggatcaatgtttctgcacatcacgccgataagggcgcacggtttgcatggttatcaccgttcggaaaacaccgcggcgtccctgtcacggtgtcggacaaattgtcataactgcgacacaggagtttgcgatgaccctgaatatgatgctcgataacgccgtacccgaggcgattgccggtgcgctgactcaacaacatccggggctgttttttacaatggtcgaacaggcatcggtagcgatttccctcaccgatgcccgggcgaatattacctacgccaacccggcgttttgccgccagactggatactcgctggcgcaattgctcaatcaaaacccgcgcctgctggccagcagccagacgccgcgcgagatctaccaggagatgtggcaaaccctgctccagcgccagccgtggcgcggtcagctaattaatcaggcccgcgacggcggcctgtatctggtagatatcgatatcacgccggtgctgaatccgcagggcgagctggagcattatctggcgatgcagcgggatatcagcgtcagctataccctggaacagcggctgcgcaatcatatgacgctaatggaagcggtgctcaataacatccccgccgccgtggtcgtggtcgatgagcaggatcgggtggtgatggataatctcgcctacaaaacgttctgcgcggactgcggcgggaaagagctgctggtcgagctccaggtttccccgcgcaaaatggggcccggcgcggagcaaatcctgccggtggtggttcgcggcgcggtccgctggctgtcggtaacctgctgggcgctgcccggcgtgagtgaagaagccagccgctacttcgtcgacagcgccccggcgcgcacgctgatggtgatcgccgactgtacccagcagcgccagcagcaggagcagggccggctcgaccgtctgaaacagcaaatgaccgccggtaagctgctggccgcgattcgcgagtcgctggacgcggcgctgattcagcttaattgcccaatcaatatgctggcggcggcccgccggctgaacggcgaaggcagcggcaacgtggcgctggacgcggcgtggcgcgaaggtgaagaggccatggcgcgcctgcagcgctgccgcccttctcttgagctggaaagcaatgccgtctggccgcttcagcccttttttgacgacctgtacgccctctaccgcacccgctttgacgatcgcgcgcggctgcaggtggacatggcatcgccgcatctggtcggcttcggccagcgtacccagctgctggcctgcttgagtttatggctcgaccggacgctggccctcgccgccgagctgccctccgtaccgctggagatcgagctttacgccgaagaggacgagggctggctctctttgtatctcaacgacaatgtcccgctgctgcaggtgcgctacgcccactcccccgatgccctaaactctcccggcaaagggatggagctgcggctgatccaaacgctggtcgcctaccaccgcggcgcgattgaactggcttcgcgaccgcagggaggcaccagcctggttctgcgtttcccgctctttaataccctgaccggaggtgagcaatgatccataaatccgattcggacaccaccgtcagacgtttcgatctctcccagcagtttaccgccatgcagcggataagcgtggtcctgagtcgcgccaccgaagcgagcaaaaccctgcaggaggttctgagcgtgctacataacgatgcctttatgcagcacgggatgatttgcctgtacgacagccagcaggagatcctgagcatcgaagcgctgcagcaaacggaagatcagacgctgcccggcagtacgcaaattcgctaccggccgggggaaggattagtcggtaccgtgctggcgcagggccagtcgctggtgctgccgcgcgtcgccgacgaccagcgttttctcgatcgtctgagcctgtacgactatgacctgccgtttatcgccgttccgctgatgggcccccactcccggcccatcggcgtactggcggcgcacgcgatggcgcgtcaggaagagcggctgcccgcctgcacgcgctttctcgaaaccgtcgccaatctgatcgcccagacgattcgcctgatgatcctgccaacctccgccgcgcaggcgccgcagcagagccccagaatagagcgcccgcgcgcctgtaccccttcgcgcggtttcggcctggaaaatatggtcggtaaaagcccggcgatgcggcagattatggatattattcgtcaggtttcccgctgggataccacggtgctggtacgcggcgagagcggcaccgggaaagagctcatcgccaacgccatccaccataattctccgcgcgccgccgcggcgttcgtcaaatttaactgcgcggcgctgccggacaacctgctggagagcgagctgtttggtcatgagaaaggcgcgtttaccggcgcggtgcgccagcggaaaggccgctttgagctggcggacggcggcaccttattcctcgatgagatcggcgaaagcagcgcctcgtttcaggctaagctactgcgtattctgcaagagggggagatggagcgcgtcggcggcgacgaaaccctgcgggtcaacgtgcgcattatcgcggcgaccaaccgccatctggaagaggaggtgcggctgggtcatttccgcgaggatctatactaccgcctgaacgtaatgcctatcgcgctgccgccgctgcgcgagcgccaggaggatatcgccgagctggcgcactttctggtgcgaaaaatcgcccacagccaggggcgaacgctgcgcatcagcgatggggcgattcgcctgctgatggagtacagctggccgggaaacgtgcgcgaactggaaaactgtctcgaacgttcggcggtgctgtcggaaagcggcctgatagaccgggacgtgattctgttcaaccatcgcgataacccgccgaaagcgctcgccagcagcggcccggcggaggacggctggctcgataacagcctcgacgagcgccagcggctgatcgccgccctggaaaaagcgggctgggtgcaggccaaagcggcgcggctgctcggcatgaccccgcgccaggtggcgtatcgcattcagattatggatatcaccatgccgcgactgtgaagccttatgtgagattcaggacattgtcgccagcgcggcggaattgcgacaattcagggacgcgggttgccggttaaaaagtctacttttcatgcggttgcgaaattaacctctggtacagcatttgcagcaggaaggtatcgcccaaccacgaaggtacgaccatgacttcctgctcctctttttctggcggcaaagcctgccgcccggcggatgacagcgcattgacgccgcttgtggccgataaagctgccgcgcacccctgctactctcgccatgggcatcaccgtttcgcgcggatgcatctgcccgtcgcgcccgcctgcaatttgcagtgcaactactgtaatcgcaaattcgattgcagcaacgagtcccgccccggggtatcgtcaacgctgctgacgcctgaacaggcggtcgtgaaagtgcgtcaggtcgcgcaggcgatcccgcagctttcggtggtgggcatcgccgggcccggcgatccgctcgccaatatcgcccgcacctttcgcaccctggagctgatccgcgaacagctgccggacctgaaattatgcctgtcgaccaacggactggtgctgcctgacgcggtggaccgcctgctggatgtcggcgttgaccacgtcacggtcaccattaacaccctcgacgcggagattgccgcgcaaatctacgcctggctatggctggacggcgaacgctacagcgggcgcgaagcgggagagatcctgattgcccgtcagcttgagggcgtacgcaggctgaccgccaaaggcgtgctggtgaaaataaattcggtgctgatccccggtatcaacgatagcggcatggccggcgtgagccgcgcgctgcgggccagcggcgcgtttatccataatattatgccgctgatcgccaggccggagcacggcacggtgtttggcctcaacggccagccggagccggacgccgagacgctcgccgccacccgcagccggtgcggcgaagtgatgccgcagatgacccactgccaccagtgtcgcgccgacgccattgggatgctcggcgaagaccgcagccagcagtttacccagcttccggcgccagagagtctcccggcctggctgccgatcctccaccagcgcgcgcagctgcacgccagcattgcgacccgcggcgaatctgaagccgatgacgcctgcctggtcgccgtggcgtcaagccgcggggacgtcattgattgtcactttggtcacgccgaccggttctacatttacagcctctcggccgccggtatggtgctggtcaacgagcgctttacgcccaaatattgtcaggggcgcgatgactgcgagccgcaggataacgcagcccggtttgcggcgatcctcgaactgctggcggacgttaaagccgtattctgcgtgcgtatcggccatacgccgtggcaacagctggaacaggaaggcattgaaccctgcgttgacggcgcgtggcggccggtctccgaagtgctgcccgcgtggtggcaacagcgtcgggggagctggcctgccgcgttgccgcataagggggtcgcctgatgccgccgctcgactggttgcggcgcttatggctgctgtaccacgcggggaaaggcagctttccgctgcgcatggggcttagcccgcgcgattggcaggcgctgcggcggcgcctgggcgaggtggaaacgccgctcgacggcgagacgctcacccgtcgccgcctgatggcggagctcaacgccacccgcgaagaggagcgccagcagctgggcgcctggctggcgggctggatgcagcaggatgccgggccgatggcgcagattatcgccgaggtttcgctggcgtttaaccatctctggcaggatcttggtctggcatcgcgcgccgaattgcgcctgctgatgagcgactgctttccacagctggtggtgatgaacgaacacaatatgcgctggaaaaagttcttttatcgtcagcgctgtttgctgcaacagggggaagttatctgccgttcgccaagctgcgacgagtgctgggaacgcagcgcctgttttgagtagccgtttcccgaagggggcgctgcaaacaaaaagccggaggtttccctccggcttttcacatcatcaaatgtgattatgcgacgtcttcgtactgcggcaccgggttgcggaagcttttggtcacgcaggcctccgtagaccagaccaataccgccccagatcaggccgagaaccatggagctctcttcgaggttaatccacagtgcgccgacggtcagcgcgccgcagaccggcagaatcagatagttgaagtggtctttcagcgttttgttgcgcttttcacggatccagaactgggagatcaccgacaggttaacgaaggtgaacgccaccagcgcgccgaggttaatcggcgccgtcgccgtgacgaggtcgagtttaatcgccagcagcgcgatcgcgcaaccagcagcacgttccatgccggagtacgccgtttcggatgcacgtagccgaagaaacgcgtcgggaacacgccgtcgcggcccatcacgtacatcagacgggaaacgcccgcgtgcgcggccgtgccggatgccagtacggtaacgctggagaaaatcagcacgccccactggaaggttttgcccgccacgtacagcatgatttcaggctgcgaggcgtccggatctttgaagcgcgagatgtccgggaagtacagctgcag

SEQ ID NO: 10 - DNA, 4983 bp, Azotobacter vinelandii
  misc_feature (3994)..(3994): n is a, c, g, or t
cccgggcccagatagggaacgatgtcgcccgagccgagctgggcgaggatttcctttaataagctgtcggtcactgaactctcctgctgagggaagggcaagaatcgacaccttattgcaataagtgtgccaagatttcgttgtttaactaattgaatttaaaagaaatcattggtgatttcggaatggcttgtcgtatccgtgggccaggatggggcgtggcttcacgacaattgtcagttttgtcacagggggccggaccaggatggtggacgctcgatggggatgtcgggccattgttcggttgtagcaattacacacatgtcggagtagggggattgtgagggggattgttgtgtatcaccccctgcagctcccgtcgatggataattaatcatttaaaatcaatggtttatttatgtgttgcgggtgctggcacagacgctgcattacctttggtgcgcggagttgttcgggcttacggccgaacgttcaagtggaaatgcaacctgaggaaattaactatggctatgcgtcaatgcgccatctacggcaaaggtggtatcggtaagtccaccactactcagaacctggtggcagccctggctgagatgggcaagaaggtcatgatcgttggttgtgacccgaaagctgactccacccgcctgatcctgcactccaaggcccagaacaccatcatggaaatggctgccgaagccggtaccgtggaagatctggagctggaagacgtgctgaaggctggctacggcggcgtcaagtgcgttgagtccggtggtccggagccgggcgttggctgcgccggccgtggtgttatcacagcaatcaacttcctggaagaggaaggcgcctacgaagacgatctggacttcgtattctacgacgtcctgggcgacgtggtgtgtggcggcttcgccatgccgatccgcgagaacaagccccaagaaatctacatcgtctgctccggtgagatgatggccatgtacgccgccaacaacatctccaagggcatcgtgaagtatgccaactccggcagcgtgcgtctgggcggcctgatctgcaacagccgtaacaccgaccgcgaagacgagctgatcatcgctctggccaacaagctgggcacccagatgatccacttcgtgccgcgtgacaacgtcgtgcagcgcgccgaaatccgccgcatgaccgtgatcgaatacgatccgaaagccaagcaagccgacgaataccgcgctctggcccgcaaggtcgtcgacaacaaactgctggtcatcccgaacccgatcaccatggacgagctcgaagagctgctgatggaattcggtatcatggaagtcgaagacgaatccatcgtcggcaaaaccgccgaagaagtctgatagccgctccggtttcagaaggactttacagggcagattggctctgtcggggtggcgccccccgcattgggcgggcgcccacccgttacccgcattatgaacgctaaggcaagaggagtcatacccatgacccgtatgtcgcgcgaagaggttgaatccctcatccaggaagttctggaagtttatcccgagaaggctcgcaaggatcgtaacaagcacctggccgtcaacgacccggcggttacccagtccaagaagtgcatcatctccaacaagaagtcccagcccggtctgatgaccatccgcggctgcgcctacgccggttccaaaggcgtggtctggggccccatcaaggacatgatccacatctcccacggtccggtaggctgcggccagtattcgcgcgccggccgtcgtaactactacatcggtaccaccggtgtgaacgccttcgtcaccatgaacttcacctcggacttccaggagaaggacatcgtgttcggtggcgacaagaagctcgccaaactgatcgacgaagtggaaaccctgttcccgctgaacaagggtatctccgtccagtccgagtgcccgatcggcctgatcggcgacgacatcgaatccgtgtccaaggtcaagggcgccgagctcagcaagaccatcgtaccggtccgttgcgaaggcttccgcggcgtttgccagtccctgggccaccacatcgccaacgacgcagtccgcgactgggtcctgggcaagcgtgacgccgacaccaccttcgccagcactccttacgatgtggccatcatcggcgactacaacatcggcggcgacgcctggtcttcccgcatcctgctggaagaaatgggcctgcgttgcgtagcccagtggtccggcgacggctacatctcccaaatcgagctgaccccgaaggtcaagctgaacctggttcactgctaccgctcgatgaactacatctcccgtcacatggaagagaagtacggtatcccatggatggagtacaacttcttcggcccgaccaagaccatcgagtcgctgcgtgccatcgccgccaagttcgacgagagcatccagaagaagtgcgaagaggtcatcgccaagtacaagcccgagtgggaagcggtggtcgccaagtaccgtccgcgcctggaaggcaagcgcgtcatgctctacatcggtggcctgcgtccgcgccacgtgatcggcgcctacgaagacctgggcatggaagtggtgggtaccggctacgagttcgcccacaacgacgactatgaccggaccatgaaagaaatgggtgactccaccctgctgtacgatgacgtgaccggcatggaattcgaagaattcgtcaagcgcatcaagcccgacctgatcggctccggtatcaaggagaagttcatcttccagaagatgggcatccccttccgtcaaatgcactcctgggattattccggcccctaccacggcttcgatggcttcgccatcttcgcccgtgacatggacatgaccctgaacaatccgtgctggaagaaactgcaggctccctgggaagcttccgaaggcgccgagaaagtcgccgccagcgcctgatagcagagcaatcgtacgcaacgtccgctgcgggcggtttccgccggccgacattccgctaacgccgttcacagatgagtgaggcgtaggagagagtcatgagccagcaagtcgataaaatcaaagccagctacccgctgttcctcgatcaggactacaaggacatgcttgccaagaagcgcgacggcttcgaggaaaagtatccgcaggacaagatcgacgaagtattccagtggaccaccaccaaggaataccaggagctgaacttccagcgcgaagccctgaccgtcaacccggccaaggcttgccagccgctgggcgccgttctctgcgccctcggtttcgagaagaccatgccctacgtgcacggttcccagggttgcgtcgcctacttccgctcctacttgaaccgtcatttccgcgagccggtttcctgcgtttccgactccatgaccgaagacgcggcagtgttcggcggccagcagaacatgaaggacggtctgcagaactgtaaggctacctacaagcccgacatgatcgcagtgtccaccacctgcatggccgaggtcatcggtgacgacctcaacgccttcatcaacaactcgaagaaggaaggtttcattcctgacgagttcccggtgccgttcgcccataccccgagcttcgtgggcagccacgtgaccggctgggacaacatgttcgaaggcattgctcgctacttcaccctgaagtccatggacgacaaggtggttggcagcaacaagaagatcaacatcgtccccggcttcgagacctacctgggcaacttccgcgtgatcaagcgcatgctttcggaaatgggcgtgggctacagcctgctctccgatccggaagaagtgctggacaccccggctgacggccagttccgcatgtacgcgggcggcaccactcaggaagagatgaaggacgctccgaacgccctcaacaccgtcctgctgcagccgtggcacctngagaagaccaagaagttcgtcgagggtacctggaagcacgaagtaccgaagctgaacatcccgatgggcctggactggaccgacgagttcctgatgaaagtcagcgaaatcagcggccagccgattccggcgagcctgaccaaggagcgtggccgtctggtcgacatgatgaccgactcccacacctggctgcacggcaagcgtttcgccctgtggggtgatccggacttcgtgatgggcctggtcaagttcctgctggaactgggttgcgagccggtacacattctctgccacaacggcaacaagcgttggaagaaggcggtcgacgccatcctcgccgcttcgccctacggcaagaatgctaccgtctacatcggcaaggacctgtggcacctgcgttcgctggtcttcaccgacaagccggacttcatgatcggcaacagctacggtaagttcatccagcgcgacaccctgcacaagggcaaggagttcgaggttccgctgatccgtatcggcttcccgatcttcgaccgtcatcacctgcatcgctccaccaccctgggttacgagggcgccatgcagatcctgaccaccctggtgaactcgatcctggaacgtctggacgaggaaacccgcggtatgcaggccaccgactacaaccacgacctggtacgctaagtcgtcggttcaagtggtatcggccggagcggcgcaagctgctctcccttggcggcggccgcaggtggtcgggccttttgcccgcgatctgcggcaaccgccaaacccgtctaaggagcaagcccatgcccagcgtcatgattcgccgcaacgacgaaggccaactgaccttctatatcgccaagaaagaccaggaagagatcgtggtgtccctggagcatgacagccccgaactctggggtggcgaagtcaccctcggcgacggttcgacctatttcatcgagccgataccgcaacccaagctgccgatc

SEQ ID NO: 11 - DNA, 150 bp, Nicotiana tabacum
gctctagttggatttgctcccccgccgtcgttcaatgagaatggataagaggctcgtgggattgacgtgagggggcagggatggctatatttctgggagcgaactccgggcgaatttgaagcgcttggatacagttgtagggagggatcc

SEQ ID NO: 12 - DNA, 210 bp, Cauliflower Mosaic Virus
tccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcacgtgttgag

SEQ ID NO: 13 - DNA, 395 bp, Nicotiana tabacum
gatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcga

SEQ ID NO: 14 - DNA, 210 bp, Cauliflower Mosaic Virus
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtga

SEQ ID NO: 15 - DNA, 792 bp, Artificial Sequence
  Spectinomycin resistance gene aadA
atgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcttgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 16 - DNA, 2686 bp, Artificial Sequence
  Cloning vector pUC19
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 224 bp, Nicotiana tabacum
gggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttacc

SEQ ID NO: 18 - DNA, 1627 bp, Nicotiana tabacum
cttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccccaaaaaaaagaagggagctacgtctgagttaaacttggagatggaagtcttctttcctttctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttagcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctgcgccagggaaaagaatagaagaagcatct

SEQ ID NO: 19 - DNA, 1625 bp, Nicotiana tabacum
actacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctttatgatagcacccaatttttccgattcggcggttcgatctatgatttatcattcatggacgttgataagatccatccatttagcagcaccttaggatggcatagccttaaaagtgaagggcgaggttcaaacgaggaaaggcttacggtggatacctaggcacccagagacgaggaagggcgtagtaatcgacgaaatgcttcggggagttgaaaataagcatagatccggagattcccgaatagggcaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagcccgaatgctgcaccctagatggcgaaagtccagtagccgaaagcatcactagcttatgctctgacccgagtagcatggggcacgtggaatcccgtgtgaatcagcaaggaccaccttgcaaggctaaatactcctgggtgaccgatagcgaagtagtaccgtgagggaagggtgaaaagaacccccatcggggagtgaaatagaacatgaaaccgtaagctcccaagcagtgggaggagccagggctctgaccgcgtgcctgttgaagaatgagccggcgactcataggcagtggcttggttaagggaacccaccggagccgtagcgaaagcgagtcttcatagg

SEQ ID NO: 20 - DNA, 6743 bp, Artificial Sequence
  Chloroplast transformation vector pCTV
gtttaaaccggtcttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccccaaaaaaaagaagggagctacgtctgagttaaacttggagatggaagtcttctttcctttctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttagcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctgcgccagggaaaagaatagaagaagcatctggcgcgccgcgaaattaatacgactcactatagggagaccacgccgtcgttcaatgagaatggataagaggctcgtgggattgacgtgagggggcagggatggctatatttctgggagcgaactccgggcgaatttgaagcgcttggatacgcatgcaggaggtatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataataggatcgtttatttacaacggaatggtatacaaagtcaacagatctcaactcgagacctcaatgaattcattggaccgcggatcaaggtaccatagatatcattagctagcactaactagtagtagtcgacatcaagagctcattccacatatgactggaggatccacaaggcctatcaaggcgccattaattaaaggccggccaatttaaatacaagcttgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcgacctgcagactacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctttatgatagcacccaatttttccgattcggcggttcgatctatgatttatcattcatggacgttgataagatccatccatttagcagcaccttaggatggcatagccttaaaagtgaagggcgaggttcaaacgaggaaaggcttacggtggatacctaggcacccagagacgaggaagggcgtagtaatcgacgaaatgcttcggggagttgaaaataagcatagatccggagattcccgaatagggcaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagcccgaatgctgcaccctagatggcgaaagtccagtagccgaaagcatcactagcttatgctctgacccgagtagcatggggcacgtggaatcccgtgtgaatcagcaaggaccaccttgcaaggctaaatactcctgggtgaccgatagcgaagtagtaccgtgagggaagggtgaaaagaacccccatcggggagtgaaatagaacatgaaaccgtaagctcccaagcagtgggaggagccagggctctgaccgcgtgcctgttgaagaatgagccggcgactcataggcagtggcttggttaagggaacccaccggagccgtagcgaaagcgagtcttcatagggcggccgcccgggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgac

SEQ ID NO: 21 - protein, 830 aa, Streptomyces thermoautotrophicus
MALPQTELRPMGKPILRKEDPRLIRGKGRFVDDILLPNMLHLCILRSPYAHARIRRIDTSKAEAAPGVKLVLTGEDLAKMNLAWMPTLAGDVQMVLATGKVLFQYQEVAAVVAETRAQAEDAIQLIEVDYEPLPVVVDPFKALEPDAPILREDKEKKSNHIWHWEAGDREETDAIFREAPVVVKQDVRFQRVHPSPLEPCGCVADYNPATGKLVVYVTSQAPHVHRTAIALTTGFPEHMIQVISPDVGGGFGNKVPLYPGYVVAIVASLKLGVPVKWIETRTENIASTHFARDYHMTAEIAATEDGKMLALRVKTIADHGAFDATANPTKYPAGLYSIVTGSYDFKAAFVEVDGVHTNKPPGGVAYRCSFRVTEASYLIERVVDVLARRLKMDPAELRLRNFIRKEQFPYRSPTGWVYDSGDYEKTFKLALERIGYEELRKEQKEKWARGEFMGIGISTFTEIVGAGPAHSFDILGIKMFDSAEIRVHPTGKVIARLGVRHQGQGHETTFAQIIAEELGLSVDDVVVEEGDTDTAPYGLGTYASRSTPTAGAAAALCARRIRDKARKIAAHLLEVNEDDVVWDGAAFSVKGLPGRSVTMKDVAFAAYTNVPDGIEPGLEASYYYNPPNLTFPYGAYIAVVDIDKGTGAVKVRRFLAVDDCGNVINPMIVEGQVHGGLTEGFAIAFMQDIPYDADGNCLAPNWMDYLVPTAWDTPQLETDRTVTPSPHHPLGAKGVGESPNVGSPAAFVNAVLDALSPLGVEHIDMPIYPWKVWKILRDTALRSDSMAIPASFQSARREKPGGGIASGPIKWTTSGRQRGRWMNARSLTSG

SEQ ID NO: 22 - protein, 171 aa, Streptomyces thermoautotrophicus
MKIRVKVNGTLYEADVEPRTLLAYFLREELKLTGTHIGCDTTTCGACTVLLDGKAVKSCTVLAVQANGREVMTVEGLEKDGQLHPLQVAFWEEHALHCGYCTPGMLMASYALLQENPMPTEEEIRFGLSGNVCRCTGYMNIVKAVQSAARRLSGASGEAVGEVATSGTAAD

SEQ ID NO: 23 - protein, 294 aa, Streptomyces thermoautotrophicus
MFPNAFKYEAPASVDEAVRLLAEYGYDGKVLAGGQSLLPMMKLRVAAPAVLIDINGIDALQGWREVDGKLRVGAMTRHAELEHAKELRDTYPLFFQTARWIADPLIRNRGTIGGSLAHADPGSDWGAAMIALRAEVEARGPQGSRLIPIDEFFVDTFATALNEDELAVAVHVPTPKGPAASRYMKLERRAGDFAIAALAVHVALGTDGRVSEAGIGICACGPIPLRAAKAEAALIGRPLTEEVIVEASRLVPEDAEPADDLRGSAEYKRDVLRVFAARALRDIAKELQGKVGIQ

SEQ ID NO: 24 - protein, 207 aa, Streptomyces thermoautotrophicus
MFELPPLPYPYDALEPYFDAKTMEIHYNGHHGAYVKNLNAALEKYPAWQNKPIEELLQSLDQLPEDIRTAVRNNGGGHYNHSFWWPMLKKNEGGQPVGKFAEAINRDFGSFEAFKDAFSKAAAGRFGSGWAWVVVEPDGKLTVTTTPNQDNPVMEGKTVVFGLDVWEHAYYLKYQNRRPEYIQAFWNVVNWDVVNERYEEALKKFGR

SEQ ID NO: 25 - DNA, 2566 bp, Artificial Sequence
  DNA segment containing sdnL gene optimized for expression in chloroplasts
ggtaccaggaggtatttatggctttgcctcaaactgaactacgacctatggggaaacccatattaaggaaagaggacccacgattaatccgaggtaagggtcgttttgttgatgatatattattaccaaatatgttacacttatgtattttaaggtccccctatgctcacgctaggatacgacgtatcgatacctcaaaagcagaggcagctcctggcgttaaattagttcttactggtgaagatttagctaaaatgaatcttgcctggatgcccactttggctggcgatgtccaaatggtcttagccacaggtaaggtactttttcaataccaagaagttgcagcagtagttgctgaaactagagcgcaggcagaggatgctattcaattaatagaagtagattatgaacctttgcctgtggtagtagatccctttaaagctcttgaaccagacgctccaatcttacgtgaagataaagaaaaaaaatcaaatcatatctggcattgggaggccggtgatagagaagaaacagatgctatatttcgagaggcccctgtggttgtaaaacaagatgtacgatttcaaagagttcatccctccccacttgaaccttgtggatgtgtcgctgattacaatccagctactggaaaacttgtagtatatgttacgtcacaagcgccacatgtacatagaacagcaattgcattgaccacaggatttccagaacacatgatacaggttattagtccggatgtagggggtggattcggaaataaagttcctctttatcctggttatgttgtggctattgtagcatctttaaaattaggtgttcctgttaaatggattgagaccagaacggaaaatattgcttctacacattttgccagagactatcacatgaccgctgaaattgccgctacggaagatggtaaaatgttagcccttcgtgttaaaacaattgctgatcatggtgcctttgacgctacagctaatcctaccaaatatcctgctggactttactctatagttacaggaagttacgactttaaggcagcctttgttgaagtagatggtgtacacactaacaaacctccgggaggcgtagcctaccgatgctcctttagagttacagaagcgagttatttgatagaacgagtggttgatgtcttggctagacgattaaaaatggaccccgctgaattaagactaaggaacttcattcgtaaggagcaatttccttatagaagtcccactggctgggtatacgattcaggtgattatgaaaaaacgttcaaattagctcttgagagaatagggtatgaagaactacgtaaagagcaaaaagaaaaatgggctagaggagaatttatgggtatcggcatcagtacttttacagaaattgtgggagcaggaccagcccattcattcgatatattagggataaaaatgttcgattcagcagaaatcagagtgcatcctaccggaaaggttattgctcgtttaggtgttagacatcagggccaaggtcatgagacaacttttgcacaaattattgcagaagaacttggcctttcagttgatgatgttgtagtagaggagggtgatacggatacagcgccttatggacttggaacctatgcctctcgaagtacaccaactgccggggcagctgcggctttgtgtgctcgaagaattagagataaagcaagaaaaatcgcagctcatcttcttgaggtaaacgaagacgatgtagtatgggatggcgcagctttttctgtgaaaggtttaccaggacgttctgtcactatgaaggatgtagcatttgctgcctataccaatgtgccagatggcatcgaaccgggtctagaggctagttattattataatccgccaaacttaacttttccttatggtgcctacatagcagtcgttgacattgataaaggaactggagcggttaaagtacgaagatttttagctgtagatgattgcggaaatgtaataaatccgatgatagtagaaggacaagtccatgggggtttaacagaaggttttgcaatagcgtttatgcaagatataccttatgatgcagatgggaactgtctagctcctaattggatggattaccttgtaccaacggcatgggatactccgcaattagagacagatagaactgtgacccctagtcctcatcatcctttgggagcaaaaggagttggagagtctcccaatgtcggatctcccgccgcattcgtaaatgctgttctagatgccctatctccactaggtgtagaacatattgatatgcctatttatccttggaaagtctggaaaatattacgagacaccgcccttcgttctgattctatggctattccagcttctttccaaagtgcacgacgagagaaacctggcggaggtattgcatctggacccattaagtggactacatctggacgtcaacgagggagatggatgaatgctcgttctttaacttctggctaataggatcgtttatttacaacggaatggtatacaaagtcaacagatctcaagctagc

SEQ ID NO: 26 - DNA, 2220 bp, Artificial Sequence
  DNA segment containing sdnS - sdnM - sdnO genes optimized for expression in chloroplasts
gctagcaggaggtatttatgaaaattagagtaaaggttaacggaaccttatatgaagctgatgttgaaccgcgtaccttattggcttatttcttacgtgaagaacttaaattaacgggcactcatattggatgtgatacgacaacttgcggggcttgtactgtactacttgatggaaaagcggttaaatcttgcactgtactagccgtacaagctaacggcagagaggttatgacagtggaaggacttgaaaaggatggtcaacttcatcctttacaggttgctttttgggaggaacatgccctacattgtggatactgtacacccggtatgttgatggctagttatgctttgttacaggaaaatccgatgccgaccgaggaagagattagattcggactttcagggaatgtttgtcgatgtactggctatatgaatatagtcaaagctgtacaatcagcagcaagacgtcttagtggagcttctggtgaagctgttggagaggtagcaacttctggcactgctgctgactaataggatcgtttatttacaacggaatggtatacaaagtcaacagatctcaaaggaggtatttatgtttcccaatgccttcaaatatgaggctccagcttcagtagatgaagcagtacgtctattagccgagtatggatatgatggtaaggttttagctggcggtcaatccttgctacctatgatgaaactacgagtcgctgctcctgccgtacttattgatataaatggtattgatgcgttacaaggatggcgtgaagttgatgggaaattacgtgtcggagccatgacacgtcatgcggaattagaacatgcaaaagagcttagggatacttatcctttgttcttccaaactgcgcgttggattgctgatccgttaatccgaaatagaggaacaattggaggaagtctagctcatgctgatccagggtctgactggggggcagcaatgattgctttacgagctgaggtggaagcccgtggtcctcaagggtctcgtttaattcccattgacgaattttttgttgatacttttgccaccgctttaaatgaggatgaattggccgttgccgtacatgtaccgacacctaaagggcctgctgcatcacgatacatgaaactagaacgtcgagcaggtgattttgctatagccgctttggcagtacatgtcgcattaggtacagatggtcgtgtctctgaagctggtattgggatatgtgcttgtggtcccattccgctaagagccgccaaagctgaagcggctttgatcggacgtcccttaactgaagaagtaatagtagaagcgtctagattggttccagaagatgctgaacctgccgatgacttacgaggttctgccgaatataaacgagatgtacttagggtattcgccgcccgagctttaagagatatagcaaaagaacttcagggcaaggttggaatacaataataggatcgtttatttacaacggaatggtatacaaagtcaacagatctcaaaggaggtatttatgtttgaattaccacctttaccatatccgtacgacgctttggaaccgtatttcgatgcaaagactatggaaattcattataatggtcatcacggtgcatacgtcaagaatctaaatgctgctttagaaaagtatcctgcctggcaaaataagcccattgaagaattattgcaatctttagatcagttaccggaagatattcgtactgctgttcgaaataacggaggcggacattataaccatagtttttggtggcctatgttgaaaaagaatgaggggggtcaacctgtaggaaaatttgccgaagctataaatcgtgattttggtagttttgaagcgtttaaggatgctttttccaaagccgcagctgggcgttttggatctggctgggcttgggttgtagttgagccggatggaaaattaacggtcaccacaactcccaatcaagataatcctgttatggaagggaagactgtagtgtttggtttggatgtttgggaacatgcttattatttaaaatatcaaaatagacgtccggaatacatacaggctttttggaatgtcgtaaattgggatgtagtaaatgaacgatatgaagaagctctaaaaaaattcggccgttaataggatcgtttatttacaacggaatggtatacaaagtcaacagatctcaacatatg

SEQ ID NO: 27 - DNA, 11455 bp, Artificial Sequence
  pCTV-StNitrogenase vector
gtttaaaccggtcttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccccaaaaaaaagaagggagctacgtctgagttaaacttggagatggaagtcttctttcctttctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttagcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctgcgccagggaaaagaatagaagaagcatctggcgcgccgcgaaattaatacgactcactatagggagaccacgccgtcgttcaatgagaatggataagaggctcgtgggattgacgtgagggggcagggatggctatatttctgggagcgaactccgggcgaatttgaagcgcttggatacgcatgcaggaggtatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataataggatcgtttatttacaacggaatggtatacaaagtcaacagatctcaactcgagacctcaatgaattcattggaccgcggatcaaggtaccaggaggtatttatggctttgcctcaaactgaactacgacctatggggaaacccatattaaggaaagaggacccacgattaatccgaggtaagggtcgttttgttgatgatatattattaccaaatatgttacacttatgtattttaaggtccccctatgctcacgctaggatacgacgtatcgatacctcaaaagcagaggcagctcctggcgttaaattagttcttactggtgaagatttagctaaaatgaatcttgcctggatgcccactttggctggcgatgtccaaatggtcttagccacaggtaaggtactttttcaataccaagaagttgcagcagtagttgctgaaactagagcgcaggcagaggatgctattcaattaatagaagtagattatgaacctttgcctgtggtagtagatccctttaaagctcttgaaccagacgctccaatcttacgtgaagataaagaaaaaaaatcaaatcatatctggcattgggaggccggtgatagagaagaaacagatgctatatttcgagaggcccctgtggttgtaaaacaagatgtacgatttcaaagagttcatccctccccacttgaaccttgtggatgtgtcgctgattacaatccagctactggaaaacttgtagtatatgttacgtcacaagcgccacatgtacatagaacagcaattgcattgaccacaggatttccagaacacatgatacaggttattagtccggatgtagggggtggattcggaaataaagttcctctttatcctggttatgttgtggctattgtagcatctttaaaattaggtgttcctgttaaatggattgagaccagaacggaaaatattgcttctacacattttgccagagactatcacatgaccgctgaaattgccgctacggaagatggtaaaatgttagcccttcgtgttaaaacaattgctgatcatggtgcctttgacgctacagctaatcctaccaaatatcctgctggactttactctatagttacaggaagttacgactttaaggcagcctttgttgaagtagatggtgtacacactaacaaacctccgggaggcgtagcctaccgatgctcctttagagttacagaagcgagttatttgatagaacgagtggttgatgtcttggctagacgattaaaaatggaccccgctgaattaagactaaggaacttcattcgtaaggagcaatttccttatagaagtcccactggctgggtatacgattcaggtgattatgaaaaaacgttcaaattagctcttgagagaatagggtatgaagaactacgtaaagagcaaaaagaaaaatgggctagaggagaatttatgggtatcggcatcagtacttttacagaaattgtgggagcaggaccagcccattcattcgatatattagggataaaaatgttcgattcagcagaaatcagagtgcatcctaccggaaaggttattgctcgtttaggtgttagacatcagggccaaggtcatgagacaacttttgcacaaattattgcagaagaacttggcctttcagttgatgatgttgtagtagaggagggtgatacggatacagcgccttatggacttggaacctatgcctctcgaagtacaccaactgccggggcagctgcggctttgtgtgctcgaagaattagagataaagcaagaaaaatcgcagctcatcttcttgaggtaaacgaagacgatgtagtatgggatggcgcagctttttctgtgaaaggtttaccaggacgttctgtcactatgaaggatgtagcatttgctgcctataccaatgtgccagatggcatcgaaccgggtctagaggctagttattattataatccgccaaacttaacttttccttatggtgcctacatagcagtcgttgacattgataaaggaactggagcggttaaagtacgaagatttttagctgtagatgattgcggaaatgtaataaatccgatgatagtagaaggacaagtccatgggggtttaacagaaggttttgcaatagcgtttatgcaagatataccttatgatgcagatgggaactgtctagctcctaattggatggattaccttgtaccaacggcatgggatactccgcaattagagacagatagaactgtgacccctagtcctcatcatcctttgggagcaaaaggagttggagagtctcccaatgtcggatctcccgccgcattcgtaaatgctgttctagatgccctatctccactaggtgtagaacatattgatatgcctatttatccttggaaagtctggaaaatattacgagacaccgcccttcgttctgattctatggctattccagcttctttccaaagtgcacgacgagagaaacctggcggaggtattgcatctggacccattaagtggactacatctggacgtcaacgagggagatggatgaatgctcgttctttaacttctggctaataggatcgtttatttacaacggaatggtatacaaagtcaacagatctcaagctagcaggaggtatttatgaaaattagagtaaaggttaacggaaccttatatgaagctgatgttgaaccgcgtaccttattggcttatttcttacgtgaagaacttaaattaacgggcactcatattggatgtgatacgacaacttgcggggcttgtactgtactacttgatggaaaagcggttaaatcttgcactgtactagccgtacaagctaacggcagagaggttatgacagtggaaggacttgaaaaggatggtcaacttcatcctttacaggttgctttttgggaggaacatgccctacattgtggatactgtacacccggtatgttgatggctagttatgctttgttacaggaaaatccgatgccgaccgaggaagagattagattcggactttcagggaatgtttgtcgatgtactggctatatgaatatagtcaaagctgtacaatcagcagcaagacgtcttagtggagcttctggtgaagctgttggagaggtagcaacttctggcactgctgctgactaataggatcgtttatttacaacggaatggtatacaaagtcaacagatctcaaaggaggtatttatgtttcccaatgccttcaaatatgaggctccagcttcagtagatgaagcagtacgtctattagccgagtatggatatgatggtaaggttttagctggcggtcaatccttgctacctatgatgaaactacgagtcgctgctcctgccgtacttattgatataaatggtattgatgcgttacaaggatggcgtgaagttgatgggaaattacgtgtcggagccatgacacgtcatgcggaattagaacatgcaaaagagcttagggatacttatcctttgttcttccaaactgcgcgttggattgctgatccgttaatccgaaatagaggaacaattggaggaagtctagctcatgctgatccagggtctgactggggggcagcaatgattgctttacgagctgaggtggaagcccgtggtcctcaagggtctcgtttaattcccattgacgaattttttgttgatacttttgccaccgctttaaatgaggatgaattggccgttgccgtacatgtaccgacacctaaagggcctgctgcatcacgatacatgaaactagaacgtcgagcaggtgattttgctatagccgctttggcagtacatgtcgcattaggtacagatggtcgtgtctctgaagctggtattgggatatgtgcttgtggtcccattccgctaagagccgccaaagctgaagcggctttgatcggacgtcccttaactgaagaagtaatagtagaagcgtctagattggttccagaagatgctgaacctgccgatgacttacgaggttctgccgaatataaacgagatgtacttagggtattcgccgcccgagctttaagagatatagcaaaagaacttcagggcaaggttggaatacaataataggatcgtttatttacaacggaatggtatacaaagtcaacagatctcaaaggaggtatttatgtttgaattaccacctttaccatatccgtacgacgctttggaaccgtatttcgatgcaaagactatggaaattcattataatggtcatcacggtgcatacgtcaagaatctaaatgctgctttagaaaagtatcctgcctggcaaaataagcccattgaagaattattgcaatctttagatcagttaccggaagatattcgtactgctgttcgaaataacggaggcggacattataaccatagtttttggtggcctatgttgaaaaagaatgaggggggtcaacctgtaggaaaatttgccgaagctataaatcgtgattttggtagttttgaagcgtttaaggatgctttttccaaagccgcagctgggcgttttggatctggctgggcttgggttgtagttgagccggatggaaaattaacggtcaccacaactcccaatcaagataatcctgttatggaagggaagactgtagtgtttggtttggatgtttgggaacatgcttattatttaaaatatcaaaatagacgtccggaatacatacaggctttttggaatgtcgtaaattgggatgtagtaaatgaacgatatgaagaagctctaaaaaaattcggccgttaataggatcgtttatttacaacggaatggtatacaaagtcaacagatctcaacatatgactggaggatccacaaggcctatcaaggcgccattaattaaaggccggccaatttaaatacaagcttgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcgacctgcagactacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctttatgatagcacccaatttttccgattcggcggttcgatctatgatttatcattcatggacgttgataagatccatccatttagcagcaccttaggatggcatagccttaaaagtgaagggcgaggttcaaacgaggaaaggcttacggtggatacctaggcacccagagacgaggaagggcgtagtaatcgacgaaatgcttcggggagttgaaaataagcatagatccggagattcccgaatagggcaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagcccgaatgctgcaccctagatggcgaaagtccagtagccgaaagcatcactagcttatgctctgacccgagtagcatggggcacgtggaatcccgtgtgaatcagcaaggaccaccttgcaaggctaaatactcctgggtgaccgatagcgaagtagtaccgtgagggaagggtgaaaagaacccccatcggggagtgaaatagaacatgaaaccgtaagctcccaagcagtgggaggagccagggctctgaccgcgtgcctgttgaagaatgagccggcgactcataggcagtggcttggttaagggaacccaccggagccgtagcgaaagcgagtcttcatagggcggccgcccgggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgac

SEQ ID NO: 28 - DNA, 22 bp, Artificial Sequence
  Primer P1
caaaatagaatactcaatcatg

SEQ ID NO: 29 - DNA, 20 bp, Artificial Sequence
  Primer P2
agatatagcaaaagaacttc

SEQ ID NO: 30 - DNA, 20 bp, Artificial Sequence
  Primer P3
cgtggtgattgatgaaactg

(19) United States
(12) Patent Application Publication
(10) Pub. No.: US 2017/0335294 A1
(43) Pub. Date: Nov. 23, 2017

(54) PLANTS CAPABLE OF NITROGEN FIXATION

(71) Applicant: Nitrogenics LLC, St. Louis, MO (US)

(72) Inventor: Alexander KRICHEVSKY, St. Louis, MO (US)

(21) Appl. No.: 15/309,713

(22) PCT Filed: May 4, 2015
(86) PCT No.: PCT/US2015/029044

§ 371 Date: Nov. 8, 2016

Related U.S. Application Data
(60) Provisional application No. 62/091,046, filed on Dec. 12, 2014.
(60) Provisional application No. 62/008,597, filed on Jun. 6, 2014.
(60) Provisional application No. 61/991,103, filed on May 9, 2014.

(51) Int. Cl. C12N 9/02; C12N 15/82

(52) U.S. Cl. CPC C12N 9/0095; C12N 15/8243; C12Y 118/06001

(57) ABSTRACT

Present invention discloses plants and plant cells comprising Streptomyces thermoautotrophicus nitrogenase and capable able of nitrogen fixation. Methods to generate said plants and plant cells are disclosed. This invention is instrumental for producing plants, including agriculturally important crops, with reduced or abolished requirements for nitrogen fertilizer.

### CROSS REFERENCE TO RELATED APPLICATIONS

This application claims benefit under 35 USC §119(e) of U.S. Provisional Application No. 61/991,103 filed on May 9, 2014, U.S. Provisional Application No. 62/008,597, filed on Jun. 6, 2014, and U.S. Provisional Application No. 62/091,046, filed on Dec. 12, 2014, the contents of each are herein incorporated by reference in their entirety.

### INCORPORATION OF SEQUENCE LISTING

The present application is being filed along with a Sequence Listing in electronic format. The Sequence Listing is provided as a file entitled, “KRI0001_401_PC_Sequence_Listing_20150504”, created May 4, 2015, which is 104,033 bytes in size. The information in the electronic format of the Sequence Listing is incorporated herein by reference in its entirety.

### BACKGROUND OF THE INVENTION

### Field of the Invention

Nitrogen fixation.

### Technical Background

Nitrogen fixation is one of the key processes required for life on Earth. Nitrogen is an essential building block for basic biological molecules, such as DNA and proteins. While 78% of the Earth's atmosphere is comprised of nitrogen gas (N₂), most organisms, plants and animals included, are unable to directly utilize atmospheric nitrogen for metabolic purposes as it must first be converted into a water soluble compound. In the nitrogen fixation process, molecular nitrogen is reduced to water-soluble form (for ex. ammonia), and becomes available for use by living organisms (Rees et al, Philos Transact A Math Phys Eng Sci, 2005, 363: 971-984).

In nature, one of the key sources of bioavailable nitrogen are diazotrophs, microorganisms converting atmospheric nitrogen into soluble nitrogenous compounds through the nitrogen fixation process. To increase nitrogen bioavailability, some plants (e.g. legumes) are known for their ability to establish symbiosis with nitrogen fixing microorganisms (e.g. rhizobia). Other natural sources of bioavailable nitrogen are known, such as fixation by lightning or decomposition of living matter. In agriculture, nitrogen is often provided to crops in the form of fertilizer generated chemically under high temperature and pressure through the Haber-Bosch process. Approximately 10⁸ tons of nitrogen are fixed on an annual basis by the chemical industry to maintain appropriate levels of agricultural production to feed the growing world's population (Rees et al, Philos Transact A Math Phys Eng Sci, 2005, 363:971-984).

Plants are typically not capable of fixing nitrogen on their own and must rely on the aforementioned external supply sources of bioavailable nitrogen. In nature, nitrogenase is the enzyme responsible for biological fixation of nitrogen (Rees et al, Philos Transact A Math Phys Eng Sci, 2005, 363: 971-984; Cheng Q, J Int Plant Biol, 2008, 50(7):784-96). A variety of nitrogenases from different organisms are known in the art. Transgenic plants comprising a nitrogenase, and thus capable of fixing nitrogen on their own, would greatly enhance agricultural productivity and reduce costs due to decrease or elimination of nitrogen fertilizer use. The reduction of nitrogen fertilizer use will also decrease the harsh impacts of fertilizer run-offs on the environment and human health. Thus it would be of great economic and social benefit to generate plants capable of fixing nitrogen on their own.

For over a century, since the discovery of nitrogen fixation, myriads of scientists and laymen alike contemplated and prophesized about creating plants capable of nitrogen fixation using essentially any known biological mechanism and a nitrogenase system. However, as of today, no one has ever been able to create such plants.

### Related Art

In the mid 1990's, Meyer's group published a number of articles with an initial description of Streptomyces thermoautotrophicus nitrogenase system: (i) Gadkari et al, Appl Environ Microbiol, 1990, 56(12):3727-34; (ii) Gadkari et al, J Bacteriol, 1992, 174(21):6840-3; and (iii) Ribbe et al, J Biol Chem, 1997, 272(42):26627-33. However, these publications neither contemplated, demonstrated nor enabled the use of Streptomyces thermoautotrophicus nitrogenase in plants or plant cells. This work provided only initial and very limited information in regard to the biochemistry, biology, genetics or functionality of Streptomyces thermoautotrophicus nitrogenase, and shed no light on its compatibility to other biological systems.

Patent application US 2014/0011261, by Wang et al., prophetically contemplates the use of prokaryotic nif encoded nitrogenases, specifically driven by T7 promoter(s), in eukaryotic cells to hypothetically enable nitrogen fixation. Yet another application US 2014/0196178, by Zaltsman, also prophetically proposes the use of nif genes to generate plants capable of nitrogen fixation. These publications have no bearing or relation to the present invention as nif nitrogenase system and genes have no biochemical, mechanistic, genetic, evolutionary, or any other relation to Streptomyces thermoautotrophicus nitrogenase, which is well known in the art to be an exceptional and unusual mechanism for nitrogen fixation (for ex. see Giller and Mapfumo, Encyclopedia of Soil Science, 2006 by Taylor & Francis). Moreover, Streptomyces thermoautotrophicus and its nitrogenase system are not disclosed or contemplated in these prophetic applications.

Nitrogen fixation is a very broad field of science with a large amount of data collected. Hence there certainly are additional patents, applications and publications in this field, such as for example the work of Cocking (U.S. Pat. No. 7,470,427 and related art), contemplating the use of bacteria living intracellularly within plant cells to enable plants to fix nitrogen. However, these works, similarly to the other publications mentioned herein, have no relation or bearing on the present invention besides all being affiliated with the broad field of nitrogen fixation.

Importantly, within the past few years, Streptomyces thermoautotrophicus nitrogenase system came under strong skepticism in the art. For example, presenters at the 18th International Congress on Nitrogen Fixation in Miyazaki, Japan, suggested that heterologous expression of Streptomyces thermoautotrophicus nitrogenase only yields hydrogen production and not necessarily fixes nitrogen. This and other occurrences have led to a prevailing belief in the field—fueled by failures of others, which in the scientific community typically not published but rather communicated directly between scientists in meetings and conferences—that Streptomyces thermoautotrophicus nitrogenase is not fit for nitrogen fixation. Additional examples of such communications are well known to those skilled in the art, for instance, a presentation by a large group of scientists at the 11th European Nitrogen Fixation Conference entitled “The genome of Streptomyces thermoautotrophicus does not contain sequences of classical or non-classical nitrogenases and three independent isolates do not fix nitrogen” by Drew MacKellar and Pamela Silver of Harvard Medical School, Boston, Mass., USA; Tony Bolger, Bjorn Usadel and Jurgen Prell of RWTH Aachen University, Aaachen, Germany; Cory Tobin of California Institute of Technology, Calif., USA; James Murray and Bill Rutherford of Imperial College, London, UK; Lucas Lieber of Universidad Nacional de Rosario, Argentina; Jeffery Norman and Maren Friesen of Michigan State University, Mich., USA.

### SUMMARY OF THE INVENTION

For many decades there was a long-felt and persistent need to create plants capable of nitrogen fixation. This concept has even been referred by those skilled in the art as the “holy grail” of agricultural biotechnology. Yet, despite multiple attempts well known to those skilled in the art, until the present invention, no one was able to create plants capable of nitrogen fixation.

The instant invention daringly defies currently accepted perception and state of the art in the field, showing that, unexpectedly and conversely to what is presently known to those skilled in the art, Streptomyces thermoautotrophicus nitrogenase can be used to fix nitrogen, and further can be used to generate plants capable of fixing nitrogen.

Nitrogenases are present in certain organisms in the nature, but not in plants. These enzymes are typically found in specialized organisms and tissues which evolved the capacity to carry out nitrogen fixation (Rees et al, Philos Transact A Math Phys Eng Sci, 2005, 363:971-984). No example, particularly of higher plants capable of nitrogen fixation on their own (e.g. without symbionts), is known in nature today.

For over a century many have dreamed, prophesized and speculated in regards to prospects and benefits of generating plants capable of nitrogen fixation on their own. Essentially all and any known nitrogen fixation system has been a subject to these desires and dreams. However, despite multiple efforts and constant trial, as of today no one has yet been able to create plants capable of fixing nitrogen on their own and bring this concept to reality.

The present invention has been uncovered as a surprising and unexpected result of an attempt to study and further characterize certain parts (St1 and St2, but not St3) of Streptomyces thermoautotrophicus nitrogenase system. Very limited data is available in regards to Streptomyces thermoautotrophicus nitrogenase as of today and additional information is needed to better understand its functionality. As described herein, chloroplast expression, amongst other expression systems considered, was preferred due to its capacity to produce large amounts of recombinant proteins at relatively low cost. This system can be used to express and purify Streptomyces thermoautotrophicus polypeptides of St1 and St2 complexes to study their properties, including purification and detection with anti-nitrogenase (S. thermoautotrophicus) antibodies, enzyme kinetics, functionality at different temperatures, prospective additional molecular partners and cofactors, crystallography studies, and many other aspects. It was not expected that St1 and St2 expressing plants would be able to fix nitrogen directly due to well-known skepticism in the art (see section “Related Art”) as well as further reasons detailed below.

In one embodiment, the instant invention encompasses plants and plant cells comprising Streptomyces thermoautotrophicus nitrogenase, and plants or plant cells capable of nitrogen fixation. In another embodiment, the present invention includes heterologous cells or organisms (e.g. other than Streptomyces thermoautotrophicus) comprising nitrogenase from Streptomyces thermoautotrophicus, and wherein said organisms can also become capable of nitrogen fixation. In yet another embodiment, the present invention includes nitrogenases from other species, particularly those carrying homologs of Streptomyces thermoautotrophicus nitrogenase, i.e. enzymes with homology to carbon monoxide dehydrogenases and having the capacity to fix nitrogen, as well as nitrogenases modified, improved or enhanced via mutagenesis, directed evolution, codon optimization or other methods known in the art. The current invention encompasses any plant, plant cell, heterologous cell or organism comprising Streptomyces thermoautotrophicus nitrogenase, wherein said nitrogenase bestows the trait of nitrogen fixation to said organism, cell or plant.

In one embodiment, the present invention demonstrates, and for the first time enables, a novel and highly advantageous method where a plant becomes capable of fixing nitrogen on its own through expression of nitrogenase components directly within a plant cell. In another embodiment, the instant invention contemplates nitrogenase expression in heterologous unicellular or multicellular organisms, other than plants, which are unable to fix nitrogen on their own, thus resulting in an unusual and novel trait of nitrogen fixation in said heterologous organisms. Also, nitrogenase can be expressed in such heterologous organisms for other reasons, such as study of nitrogenase and its functions in a new cellular environment or production of nitrogenase proteins for research and educational purposes. Non-limiting examples of said heterologous organisms are prokaryotes and eukaryotes including, but not limited to bacteria, cyanobacteria, archea, fungi, protists, algae or animals, which are naturally unable of fixing nitrogen.

In one aspect, the invention relates to a plant or a heterologous cell containing an expressible heterologous nucleotide sequence comprising a nitrogenase gene or genes, wherein the heterologous nucleotide sequence is expressed to render the plant or a heterologous cell capable of fixing nitrogen. In another aspect, the present invention relates to a method of producing a plant, heterologous cell, or organism capable of fixing nitrogen on its own. This method includes transfecting a plant cell or a heterologous cell with a vector comprising an expressible heterologous nucleotide sequence of a nitrogenase gene or genes.

Streptomyces thermoautotrophicus nitrogenase system can be employed for expression in a large number of biological systems, for either generating novel cells and organisms capable of nitrogen fixation, or for protein expression purposes to further research into its functionality, or for other studies. Non-limiting examples of such organisms include mycorrhizal fungi, which can be further used as biofertilizers, or bacterial cells such as E. coli which can be used as model organisms in research or protein expression and purification, or algal cells that can be used for biofuel production.

The present invention is instrumental for producing plants, including agriculturally important crops such as corn and cotton, with reduced or abolished requirements for nitrogen fertilizer, leading to reduced costs of agricultural production. In addition, this novel technology can produce multiple environmental benefits. Reduction in nitrogen fertilizer use will decrease incidence of fertilizer run-offs, helping to reduce negative impact on water supplies, wildlife and human health. Moreover, reduced nitrogen requirements may provide an important advantage to row crops over weeds and lead to reduced use of herbicides in agriculture, further decreasing impact on the environment and human health.

Amongst its many embodiments, the present disclosure encompasses:
A plant cell comprising a nitrogenase and capable of nitrogen fixation.
A plant cell comprising Streptomyces thermoautotrophicus nitrogenase.
The plant cell of the previous embodiment, which is capable of nitrogen fixation.
A heterologous cell, naturally unable of nitrogen fixation, comprising Streptomyces thermoautotrophicus nitrogenase and capable of nitrogen fixation.
The heterologous cell of the previous embodiment, wherein said cell is a bacterial cell, a fungal cell, an algal cell or an animal cell.
A cell of any of the previous embodiments, wherein such cell can be used as biofertilizer or for biofuel production.
A cell of any of the previous embodiments transformed with a vector, wherein said vector is a plastid or a chloroplast transformation vector, a nuclear genome transformation vector, a mitochondrial genome transformation vector, or a vector maintained as an episome in said cell.
The vector of the previous embodiment, wherein said vector contains expressible nucleic acid sequence, and is a plasmid, a viral vector or any other type of vector which can be used in stable or transient transformation.
A plant cell or a heterologous cell of any of the previous embodiments comprising enhanced, optimized, codon-optimized or otherwise modified nitrogenase.
A plant cell or a heterologous cell of any of the previous embodiments, further comprising at least one cofactor for enhancing or modifying nitrogen fixation.
A plant cell or a heterologous cell of any of the previous embodiments, wherein nitrogenase further comprises a plastid or other targeting sequence.
A plant comprising Streptomyces thermoautotrophicus nitrogenase.
The plant of the previous embodiment capable of nitrogen fixation.
Progeny of plant of the previous embodiments, produced sexually or asexually.
A part of plant or progeny of the previous embodiments.
The part of the previous embodiment, which is selected from the group consisting of a protoplast, a cell, a tissue, an organ, a seed, a cutting, and an explant.
A method for making a plant capable of nitrogen fixation, comprising: transfecting at least one plant cell with at least one vector comprising at least one gene of Streptomyces thermoautotrophicus nitrogenase complex, and growing said cell into a mature plant.
A method for making a plant, a plant cell or a heterologous cell capable of nitrogen fixation, and providing means for regulating said nitrogen fixation process, comprising: transfecting at least one plant cell or heterologous cell with at least one vector comprising a gene encoding for a nitrogenase, and providing means for regulation of expression or functionality of said gene or the encoded polypeptide.
A plant, a plant cell or a heterologous cell comprising sequence homologs to Streptomyces thermoautotrophicus nitrogenase and capable of nitrogen fixation.
A plant, a plant cell or a heterologous cell comprising a nitrogenase which bears homology to a carbon monoxide dehydrogenase.
Nitrogenase crosstalk: a vector having a first heterologous nucleotide sequence comprising a nitrogenase sequence operably linked to a first promoter, and a vector having a second heterologous nucleotide sequence operably linked to a second promoter.

### BRIEF DESCRIPTION OF THE DRAWINGS

FIG. 1. Nitrogenase complex of Streptomyces thermoautotrophicus (Ribbe et al, J Biol Chem, 1997, 272 (42):26627-33), comprised of three functional complexes designated as St1, St2 and St3. St1 and St3 are heterotrimers, comprised of subunits L, M and S, whereas St2 is a homodimer comprised of D subunits. Superoxide produced by St3 through the oxidation of CO subsequently reoxidized by St2, which delivers electrons to St1, the nitrogenase. The numbers refer to the molecular weight of polypeptide subunits in kDa; MCD denotes molybdopterin cytosine dinucleotide.

FIG. 2A. Example of plant transformation system. Functional genetic elements of the Agrobacterium binary vector system. The binary vector can be constructed based on a backbone of a commonly used E. coli cloning vector, containing MCS flanked by right and left T-DNA borders (RB/LB). Once the desired selection marker and gene of interest (GOI) are cloned using standard cloning procedures, the binary vector is transferred into an Agrobacterium strain carrying helper Ti plasmid, which provides vir-encoded proteins required for the transformation process (Lee and Gelvin, Plant Physiol, 2008, 146:325-332);

FIG. 2B. Example of plant transformation system. Vectors for expression of multiple transgenes from plant nuclear genome. Multiple transgenes can be expressed from a single, or a number of vectors, used to transform a plant cell nucleus (ex. Tzfira T, Plant Mol Biol, 2005, 57(4):503-16)

FIG. 2C. Example of plant transformation system. Schematic presentation of functional features of a plastid transformation vector (Maliga, TRENDS in Biotech, 2003, 21(1):20-28). Homologous recombination machinery of the chloroplast promotes targeting of the integrating DNA into specific genomic location (e.g. LTL/RTR) via homology with sequences flanking the expression cassette. If multigene expression is desired, chloroplast polycistronic gene expression machinery allows expression of several GOIs (genes of interest) from a single operon-like structure, simplifying construction of the transformation vector and permitting integration of multiple transgenes in a single transformation step. Chloroplast and plastid transformation with multiple genes can be carried out using a single vector or a number of vectors, as known in the art.

FIG. 3A. Schematic map of chloroplast transformation vector pCTV. MCS* element within the pCTV, between the 3′ of aadA and the 5′ of TpsbA, is comprised of the following restriction sites: EcoRI-SaclI-KpnI-EcoRV-NheI-SpeI-SalI-SacI-NdeI-BamHI-StuI-KasI-PacI-FseI-SwaI-HindIII.

FIG. 3B. Schematic map of chloroplast transformation vector pCTV-StNitrogenase.

FIG. 4A. Table summarizing functional elements and expected fragments of exemplary enzymatic digests of the actual pCTV-StNitrogenase vector.

FIG. 4B. Exemplary enzymatic digests of the actual pCTV-StNitrogenase vector resolved on an ethidium bromide stained 1% agarose gel. Molecular weight marker: 1 kb DNA ladder (New England Biolabs). Schematic positioning of restriction sites in pCTV-StNitrogenase is shown in FIG. 3B and full sequence provided in SEQ ID NO: 27.

FIG. 4C. First generation of Nicotiana tabacum plants comprising Streptomyces thermoautotrophicus nitrogenase demonstrate a chimeric/heteroplastomic phenotype. Additional 2-3 cycles of regeneration of whole Nicotiana tabacum plants from leaf explants on spectinomycin supplemented media, as known in the art, were employed to obtain non-chimeric plants.

FIG. 5A. Confirmation of plants comprising Streptomyces thermoautotrophicus nitrogenase using PCR. DNA was prepared, using methods known in the art, from the leaves of aseptically grown plants transformed with pCTV-StNitrogenase, and carrying Streptomyces thermoautotrophicus nitrogenase in their genome, as well as wild type (non-transformed) plants. The DNA was used as a template in a PCR reaction with primers P1 and P2 (SEQ ID NOs: 28 and 29, respectively) and Taq polymerase (Takara). Reaction products were resolved on 1% ethidium bromide stained agarose gel. First 6 lanes from the left demonstrate formation of highly specific PCR product of correct size (approx. 1 kb) in plants containing Streptomyces thermoautotrophicus nitrogenase complex (lanes designated as “StNit plants”), while DNA from wild-type tobacco plants (two lanes following “StNit plant” lanes) failed to produce said PCR products, positively confirming presence of Streptomyces thermoautotrophicus nitrogenase in the experimental plants (“StNit plants”). First lane from the right shows 1 kb DNA ladder (New England Biolabs).

FIG. 5B. Confirmation of plants comprising GUS using PCR. PCR testing of plants generated using pCTV-GUS was conducted in a similar manner as for pCTV-StNitrogenase generated plants, except primers P1 and P3 (SEQ ID NOs: 28 and 30, respectively) were used to specifically confirm GUS gene presence. As shown, pCTV-GUS transformed plants generated the expected PCR product of correct size (approx. 1 kb; first 4 lanes from the left designated as “GUS plants”), while wild-type control plants did not (second lane from the right), positively confirming GUS presence in the transformed plants.

FIG. 5C. Confirmation of plants comprising GUS using histochemical staining. Histochemical staining using X-Gluc of GUS carrying plants (left) and wild type control plants (right). Strong histochemical staining of GUS carrying plants, but not of the control wild type plants, confirms strong expression of GUS in the transformed plants.

FIG. 6A. Plants comprising Streptomyces thermoautotrophicus nitrogenase show phenotype highly resistant to nitrogen deficiency. Typical symptoms of tobacco nitrogen deficiency appearing in foliage of 10 day old control tobacco plants, aseptically grown on N-free MSO medium (left), and manifested as “fired” appearance of the bottom leaves browning and curling at the leaf tips, but not in the experimental Streptomyces thermoautotrophicus nitrogenase comprising plants (right).

FIG. 6B. Plants comprising Streptomyces thermoautotrophicus nitrogenase show phenotype highly resistant to nitrogen deficiency. 10 day old control plants grown on N-free MSO medium demonstrated nitrogen-deficiency stimulated root growth, developing on average twice as many roots than Streptomyces thermoautotrophicus nitrogenase comprising plants (19.9 vs. 9.6 roots per plant on average, respectively).

FIG. 6C. Plants comprising Streptomyces thermoautotrophicus nitrogenase show capacity of nitrogen fixation from the air. Experimental plants comprising Streptomyces thermoautotrophicus nitrogenase (right) demonstrate approx. ˜20% increase in enrichment of 15N isotope levels, after incubation for 6-7 days in atmosphere containing 5% (vol/vol) of 15N isotope as compared to control plants (left) (average delta 15N of ˜297 vs. ˜367 for control and experimental plants, respectively).

FIG. 7A. Streptomyces thermoautotrophicus nitrogenase enables nitrogen fixation trait in a variety of plant species. Nicotiana sylvestris plants transformed with Streptomyces thermoautotrophicus nitrogenase (right-hand side of the panel, designated as “Experimental Plants”) are compared to wild-type Nicotiana sylvestris plants (left-hand side of the panel, designated as “Control Plants”) on nitorgen-free MSO medium. After 7-10 days of growth on N-free MSO medium, N. sylvestris plants carrying Streptomyces thermoautotrophicus nitrogenase retained notably greener appearance, and thus showed considerably reduced effect of nitrogen deprivation, as compared to their wild-type counterparts. Side view of the magenta box comprising: nitrogen-free MSO medium, experimental and control plants.

FIG. 7B. The top view of FIG. 7A.

### DETAILED DESCRIPTION OF THE INVENTION

The following detailed description of the invention is provided to aid those skilled in the art in practicing the present invention. Even so, the following detailed description should not be construed to unduly limit the present invention, as modifications and variations in the embodiments herein discussed may be made by those of ordinary skill in the art without departing from the spirit or scope of the present inventive discovery.

Those of ordinary skill in the art will recognize that any and all features, combinations of features, or permutations of features discussed or possible herein, including those in the description, figures, sequence listings, examples and claims, is (are) linked and are clearly and unambiguously intended to be included within the scope of the present disclosure and claims, provided that the features included in any such combination or permutation are not mutually inconsistent as will be apparent from the context, this specification, and the knowledge of one of ordinary skill in the art. Thus, additional advantages and aspects of the present invention beyond those specifically described herein will be readily apparent and enabled to those of ordinary skill in the art from the entirety of the present disclosure and claims. The contents of all publications, patent applications, patents and other references mentioned herein are incorporated by reference herein in their entirety. In case of conflict, the present specification, including explanations of terms, will control.

The term “consisting” as used herein in a claim means that the invention necessarily includes the listed ingredients, but is opened to unlisted ingredients that do not materially affect the properties of the invention. The term “comprising” in a claim herein is open-ended and means that the claim must have all the features specifically recited herein, but there is no bar on additional features that are not recited, thus leaving the claim open for the inclusion of other unspecified features. The term “consisting essentially of” in a claim is an intermediate term between claims that are written in a “consisting” format and those drafted in an open “comprising” format. All these terms can be used interchangeably herein. The use of the term “including”, as well as other related forms such as “includes” and “included”, is not limiting.

“Control” or “control level” means the level of an enzymatic or biological activity normally or typically found in nature. A control level is also referred to as a wild type or base-line level. A control plant, i.e. a plant that does not contain the recombinant DNA that confers a particular trait (ex. nitrogen fixation capacity), is used as a baseline for comparison to identify or characterize said particular trait. A suitable control plant may be a non-transgenic wild-type plant, or it may also be a transgenic plant line that comprises an empty vector, a selection marker or a marker gene, but does not contain the recombinant DNA that encodes said particular trait (ex. nitrogen fixation capacity).

The term “about” as used herein is a word with a flexible meaning akin to “nearly”. The term “about” indicates that exactitude is not claimed, but rather a contemplated variation. Thus, as used herein, the term “about” means within one or two standard deviations from the recited value, or +/− a range of up to 50%, up to 25%, up to 10%, up to 5%, or up to 4%, 3%, 2% or 1% as compared to the recited value.

As used herein, the term “Streptomyces thermoautotrophicus nitrogenase” refers to the nitrogenase of the UBT1 strain of Streptomyces thermoautotrophicus described by Ribbe et al, J Biol Chem, 1997, 272(42):26627-33 (DSMZ 41605; ATCC 49746), as well as to the nitrogenases from subspecies, varieties, strains, accessions or other close taxonomic relatives of UBT1 having the same or similar (i.e., within the range of from about 50% to about 200%, or more) of the UBT1 nitrogenase ability to fix atmospheric N₂. Similar subspecies, varieties, strains, accessions or other close relatives of UBT1 possessing such nitrogenases may include, for example, those disclosed in, but not limited to, Bergey's Manual of Systematic Bacteriology (Book 5), Springer-Verlag, second edition, 2012, pages 1554 and 1744; Kim et al, International Journal of Systematic Bacteriology, 1999, 49: 7-17; as well as accessions available from ATCC, DSM, DPDU, KCTC, NRRL, ISP, NCIM, CUB, IFO, IMSNN and other depositories or culture collections, as well as those exhibiting physical, biochemical, physiological and other characteristics consistent with those of strain UBT1 disclosed in Ribbe et al, J Biol Chem, 1997, 272(42):26627-33.

Plants and Plant Cells Comprising Streptomyces thermoautotrophicus Nitrogenase

This invention has been uncovered as a surprising and unexpected result during an attempt to study and further characterize Streptomyces thermoautotrophicus nitrogenase polypeptides. As of today, very limited data is available in regards to Streptomyces thermoautotrophicus nitrogenase system and additional information is needed to better understand its functionality. Nicotiana tabacum (tobacco) chloroplast expression system, known to produce large amounts of expressed transgenes at a very low cost, has been selected to express and purify Streptomyces thermoautotrophicus nitrogenase polypeptides (using, for example, monoclonal-antibody conjugated columns and other methods known in the art) and to study their properties, including but not limited to enzyme kinetics, stability, crystallography and other aspects. For brevity, here we demonstrate expression of Streptomyces thermoautotrophicus polypeptides in chloroplasts, which is given by way of illustration only and is not limitative of the presently disclosed embodiments. Expression of Streptomyces thermoautotrophicus polypeptides can also be achieved from nuclear or mitochondrial genomes, or episomal units, and combinations of any of the foregoing, using methods and technologies well known in the art and is not presented here for brevity.

For a host of reasons, in addition to those detailed in section “Related Art” above, it was not expected that plants carrying Streptomyces thermoautotrophicus nitrogenase would be able to fix nitrogen directly from the air. To name a few examples, Streptomyces thermoautotrophicus is an extremophile, thriving in physically extreme conditions that are detrimental to most life on Earth, and the functional temperature for its nitrogenase is 65° C. Plants function at much lower temperatures (normally 18-26° C.), at which Streptomyces thermoautotrophicus nitrogenase is not expected to work, i.e. to be functionally active in fixing of atmospheric nitrogen. Furthermore, the reaction is coupled to oxidation of carbon monoxide, found in Streptomyces thermoautotrophicus' natural environment, but not in conventional plant environments. Moreover, Streptomyces thermoautotrophicus nitrogenase is functionally dependent on St3 CO dehydrogenase (see Ribbe et al, J Biol Chem, 1997, 272(42):26627-33), found in very specific aerobic and anaerobic microbes, but which is not typical in plants, for supply of superoxide anion radicals. St3 CO dehydrogenase Cox proteins, an integral part of Streptomyces thermoautotrophicus nitrogenase system, was not expressed in the experiments with the nitrogenase parts St1 and St2 described herein, and hence the expressed partial nitrogenase complex was not expected to be functional in the absence of St3 CO dehydrogenase. Thus, according to the present invention, St1 and St2 are sufficient to catalyze nitrogen fixation in transgenic organisms such as plants. In addition, plants are extremely different in their biochemistry, genetics and other biological aspects from extremophiles like Streptomyces thermoautotrophicus. These broad biological differences manifest in vast differences in protein expression and post-translational modifications, stability, generation of correct ratio of protein complex subunits, availability of correct amounts of co-factors (Mo, Mn, Fe, etc.), and multiple other factors, rendering the possibility of Streptomyces thermoautotrophicus nitrogenase functionality in plants highly unlikely and unexpected. What's more, functional nitrogenase of Streptomyces thermoautotrophicus produces ammonia, which can be toxic to hosts such as plant cells and plants. While Streptomyces thermoautotrophicus have had an evolutionary opportunity to develop biological mechanisms to reduce toxic effects of ammonia, heterologous expression of functional Streptomyces thermoautotrophicus nitrogenase in plants was likely to result in significant cellular damage and death due to the sudden and unexpected appearance of ammonia in the cells. Thus, it was highly unexpected that plant cells could survive and thrive with a functional Streptomyces thermoautotrophicus nitrogenase.

It was noticed in the experiments herein that neglected transgenic Nicotiana tabacum plants comprising Streptomyces thermoautotrophicus nitrogenase had an extent of a different appearance than neglected wild-type plants or other transgenics. The slender difference in appearance might have resulted from differences in nutrient metabolism, and it was decided to investigate further. A very simple experiment of planting Nicotiana tabacum comprising Streptomyces thermoautotrophicus nitrogenase side by side with wild type Nicotiana tabacum on nitrogen free MSO medium was performed. Astoundingly, plants comprising Streptomyces thermoautotrophicus nitrogenase showed diminished signs of nitrogen deficiency as compared to wild type control plants, which was further determined to be a result of Streptomyces thermoautotrophicus nitrogenase activity, a highly unexpected and surprising result.

In one aspect, the present invention encompasses plants and plant cells comprising Streptomyces thermoautotrophicus nitrogenase enzyme or enzyme complex, rendering the transgenic plants capable of fixing nitrogen. In one embodiment, the plant or plant cell comprises components St1 and St2 of Streptomyces thermoautotrophicus nitrogenase (Ribbe et al, J Biol Chem, 1997, 272 (42):26627-33). Optionally, St3 component may be expressed in the same cell to enhance or modify nitrogenase activity. The nitrogenase complex of Streptomyces thermoautotrophicus, a free-living nitrogen fixing bacterium, catalyzes the following reaction:

N₂+4-12MgATP+8H⁺+8e⁻=2NH₃+H₂+4-12MgADP+4-12Pᵢ

Streptomyces thermoautotrophicus in is an extremophile which has been isolated from the covering soil of burning charcoal piles. In the reaction mediated by its nitrogenase, oxidation of carbon monoxide is coupled by a molybdenum-containing CO dehydrogenase (CODH), resulting in transfer of electrons derived from CO oxidation to oxygen and producing O₂ superoxide anion radicals. Reoxidation of the O₂ superoxide anion radicals to molecular oxygen by Mn-containing superoxide oxidoreductase is followed by transfer of the electrons by a MoFeS-dinitrogenase to N₂ and culminates in the production of ammonium ions (and ammonia) (Ribbe et al, J Biol Chem, 1997, 272 (42): 26627-33).

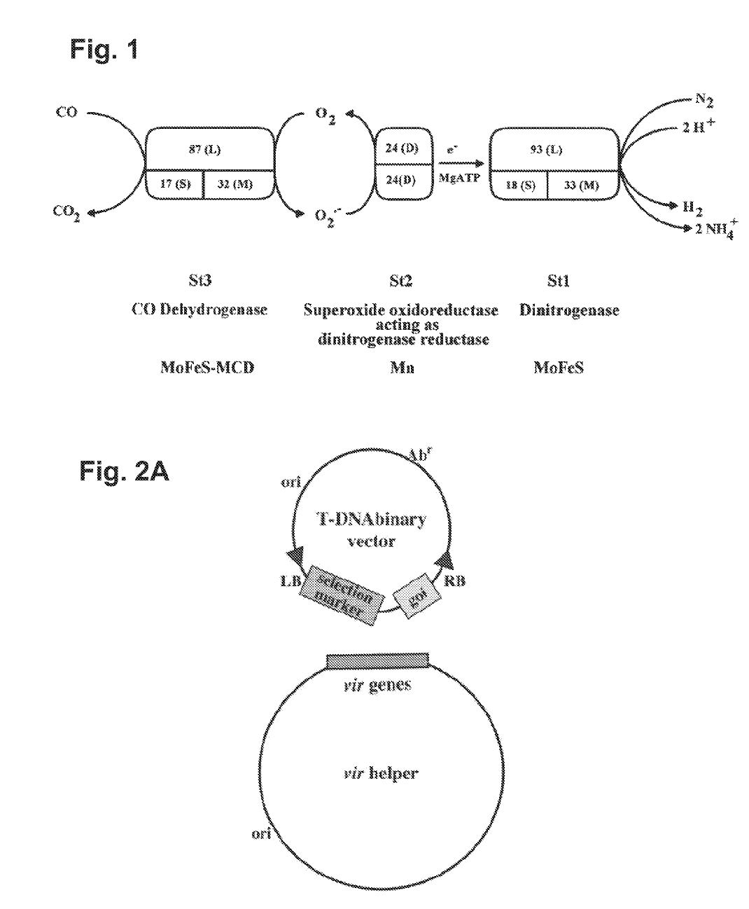

The complete Streptomyces thermoautotrophicus nitrogenase complex is comprised of components designated as St1, St2 and St3 (FIG. 1). Denaturating PAGE suggests St1 to be comprised of 3 polypeptide subunits designated as L, M and S (encoded by sdnL, sdnM and sdnS, respectively), and arranged in a heterotrimeric structure with close to a 1:1:1 subunit ratio. The St2 is a homodimer of the same type of subunit (D), encoded by sdnO. The St3 component is identified as CO dehydrogenase and is comprised of the following polypeptide subunits: CoxL, CoxM and CoxS. St3 is a molybdo-iron-sulfur-flavoprotein containing the MCD type of molybdenum cofactor (Ribbe et al, J Biol Chem, 1997, 272 (42):26627-33).

The genes coding for the nitrogenase polypeptide components (SEQ ID NOs: 5-8) can be isolated from the genome of Streptomyces thermoautotrophicus UBT1 (DSMZ 41605; ATCC 49746) (Ribbe et al, J Biol Chem, 1997, 272 (42):26627-33), or other species carrying similar nitrogenase systems. As noted above, other similar nitrogenase genes can also be isolated from subspecies, varieties, strains, accessions, or other close relatives of UBT1 having the same or similar (i.e., within the range of from about 50% to about 200%, or more) of the UBT1 nitrogenase ability to fix atmospheric N₂. Exemplary partial amino acid sequences of St1 and St2 components from Streptomyces thermoautotrophicus are shown in SEQ ID NOs: 1-4 (per Ribbe et al, J Biol Chem, 1997, 272 (42):26627-33). Exemplary full length DNA sequences of St1 and St2 components of Streptomyces thermoautotrophicus nitrogenase are shown in SEQ ID NOs: 5-8 (Genebank accession numbers: KF951061, KF951060, KF951059 and KF956113). Exemplary full length polypeptide sequences of St1 and St2 of Streptomyces thermoautotrophicus nitrogenase are shown in SEQ ID NOs: 21-24. Due to the degeneracy of the genetic code many alternate nucleic sequences to those specifically described herein can encode the nitrogenase subunits and other amino acid sequences discussed herein, and therefore those are encompassed by the present invention as well.

This invention encompasses sequence homologs of Streptomyces thermoautotrophicus nitrogenase components, which can be identified by computer data mining or sequence alignment techniques described in this specification and well known in the art. In addition, while St3 is the actual functional carbon monoxide dehydrogenase donor supplying components St1 and St2 of Streptomyces thermoautotrophicus nitrogenase (Ribbe et al, J Biol Chem, 1997, 272 (42):26627-33), component St1 itself bears a certain degree of homology to carbon monoxide dehydrogenase (CODH) types of enzymes (other names in common use include carbon-monoxide dehydrogenase, anaerobic carbon monoxide dehydrogenase, carbon monoxide oxygenase, and carbon-monoxide:(acceptor) oxidoreductase). Thus other homologs of CODH enzymes, which utilize N₂ as a substrate instead or in addition to CO gas, are functional homologues of Streptomyces thermoautotrophicus nitrogenase. Thus this invention encompasses all such enzymes, being either sequence homologs of Streptomyces thermoautotrophicus nitrogenase or being CODH enzymes capable of fixing nitrogen. In addition, component St2 bears homology to reductase types of enzymes, including superoxide dismutases (SOD), which can be used in conjunction with St1 type of enzymes. Methods to identify homologs of these types of enzymes are well known in the art, and may include data mining techniques, sequence alignment techniques, identification of homologs with similar functional domains, etc.

This invention prospectively and potentially may become applicable to other nitrogenase systems. For instance, the free-living diazotrophic bacterium Klebsiella pneumoniae possess a nitrogenase complex encoded by a cluster of nif genes (Halbleib and Ludden, J Nutr, 2000, 130:1081-4). The three structural subunits of the nitrogenase are encoded by nifHDK genes, with other nif cluster genes involved in auxiliary metabolic functions. Exemplary nif gene sequences of Klebsiella pneumoniae and Azotobacter vinelandii sequences are shown in SEQ ID NOs: 9 and 10, respectively. Additional diazotrophs are found amongst Azotobacter, Rhizobium, certain cyanobacteria, as well as other species. The instant invention contemplates the use of any nitrogenase from any organism, similarly to the use of Streptomyces thermoautotrophicus nitrogenase, which potentially and prospectively can be used in its native or modified form (for instance via mutagenesis, directed evolution, codon optimization and other techniques known in the art) to create plants, plant cells or other heterologous cells and organisms capable of nitrogen fixation.

The nucleotide sequences of the nitrogenase encoding genes may be derived from wild-type organisms. Wild-type refers to the normal gene or organism found in nature without any known mutation. Other nucleotide sequences within the invention include nucleotide sequences that encode variants of the nitrogenase genes and proteins, and nucleotide sequences that encode mutant forms, recombinant forms, or non-naturally occurring variant forms of these genes and proteins, which exhibit about 50% to about 200%, or more, of the biological/enzymatic activity of the protein in question as determined by the assays known in the art.

Heterologous Cells, other than Plant Cells, Comprising Streptomyces thermoautotrophicus Nitrogenase

Similarly to heterologous expression in plants, Streptomyces thermoautotrophicus nitrogenase can be expressed in other types of organisms and cells, which are not naturally capable of nitrogen fixation, for either generation of novel organisms capable of nitrogen fixation, or for research, or for studies of the nitrogenases. In one particular aspect of the present invention, nitrogenase (or nitrogenase complex) from Streptomyces thermoautotrophicus can be expressed in a variety of organisms where the nitrogenase, or any nitrogenase activity, is not naturally found. Non-limiting examples of desirable unicellular and multicellular organisms for such modification include bacteria (other than Streptomyces thermoautotrophicus) belonging to eubacteria and archea; cyanobacteria; fungi, including yeast, mycorrhizae, molds or mushrooms; protists and algae; or animals. Organisms particularly preferable for Streptomyces thermoautotrophicus nitrogenase expression are certain bacteria, fungi and algae. Any organism or cell type where the specific nitrogenase is not naturally found can be used for heterologous nitrogenase expression, resulting in a novel trait of nitrogen fixation in the heterologous cell or organism.

Microorganisms expressing nitrogenase and producing biologically available nitrogen are particularly useful in agriculture, for instance as biofertilizers which can be applied to soil, seed or plant surfaces. Non-limiting examples of such organisms include rhizobia, mycorrhizal fungi, pink-pigmented facultative methylotrophs (PPFM bacteria) and plant-growth promoting and plant colonizing microorganisms, for example yeast, algae, bacteria, etc. and methods of use, as known in the art. In one embodiment, these organisms express heterologous nitrogenase from Streptomyces thermoautotrophicus. By way of example only, heterologous nitrogenase from Streptomyces thermoautotrophicus can be expressed in bacteria such as E. coli, for instance, for studies of the nitrogenase complex or for expression and purification of the nitrogenase proteins. In another embodiment, nitrogenases can be expressed in unicellular or multicellular algae, which can be used as biofertilizers or in the production of biofuels. Methods for construction of transformation vectors and stably or transiently transforming unicellular and multicellular organisms, including bacteria, fungi, algae or animals, are well known in the art and are not described here for brevity.

Genetically Modified Plants, Plant Cells and Heterologous Cells Capable of Nitrogen Fixation

The terms “transgenic,” “transformed,” and “transfected” as used herein include any cell, cell line, callus, tissue, plant tissue, plant or organism into which a nucleic acid heterologous to the host cell has been introduced. The term “transgenic” as used herein does not encompass an alteration of the genome (chromosomal or extra-chromosomal) by conventional plant breeding methods or by naturally occurring events, such as random cross-fertilization, non-recombinant viral infection, non-recombinant bacterial transformation, non-recombinant transposition, or spontaneous mutation. The term “transgenic plant” refers to a plant or plant tissue that contains an inheritable heterologous nucleotide sequence. The present invention also encompasses progeny, whether produced sexually or asexually, or through breeding techniques, of plants covered by the present invention or containing sequences disclosed herein.

The term “plant” is used broadly herein to refer to a eukaryotic organism containing a plastid or plastids, and being at any stage of development. The term “plant” as used herein refers to a whole plant or a part of a plant (e.g., a plant cutting, a plant cell, a protoplast, a plant cell culture, a plant organ, a plant seed, and a plantlet), a seed, a cell- or a tissue-culture derived from a plant, plant organ (e.g., embryos, pollen, ovules, seeds, leaves, flowers, branches, fruit, kernels, ears, cobs, husks, stalks, roots, root tips, anthers, etc.), as well as unicellular or multicellular algae. Any plant may be used in this invention. This includes flowering and non-flowering plants, such as algae, monocots or dicots, and C3 and C4 plants. In one aspect for research and other purposes Arabidopsis thaliana or Nicotiana tabacum (tobacco) can be used as they are preferred model organisms in plant research. In another aspect, as marketable products and for agricultural or horticultural purposes, plants such as cotton, corn, wheat, various row crops, other food crop plants, ornamental, horticultural and other plants can be used.

Oilseed plants, e.g. plants that produce seeds or fruit with oil content from about 5% to about 50%, or more. Exemplary oilseed or oil crop plants useful in practicing the methods disclosed herein include, but are not limited to sunflower; sesame; soybean; mustard; coconut; cotton; peanut; rice; wheat; flax (linseed); sunflower; olive; corn; palm; palm kernel; sugarcane; castor bean; switchgrass, as well as, plants in the genera of Brassica (e.g., rapeseed/canola; Brassica napus; Brassica carinata; Brassica nigra; Brassica oleracea); Camelina; Jatropha (Simmondsia chinensis); Miscanthus; Borago officinalis; Ricinus communis; Coriandrum sativum; Echium plantagineum; Cuphea hookeriana; Cuphea plucherrima; Cuphea lanceolata; Crepis alpina; Crambe abyssinica; Vernonia galamensis and Momordica charanita. These include major and minor oil crops used, or being investigated and/or developed to be used as sources of biofuels due to their significant oil production and accumulation. The present invention also encompasses plants that may be used for production of biomass, for example, for biofuel production (ex. ethanol production from plant cell-wall constituents), which may include exemplary crops such as corn, soybeans, grasses, and other plants known in the art.

Agricultural plants, e.g. plants produced by agricultural practices for human food, animal feed and variety of plant products, are highly desirable targets for genetic modification with Streptomyces thermoautotrophicus nitrogenase. Examples of agricultural plants include, but not limited to, corn, cotton, soybeans, wheat, rice, tomatoes, potatoes, sugar cane, palms, beans, fruits and vegetables, sugar beet, sunflower and plethora of additional agricultural plants well known in the art.

The transgenic plant, heterologous cell or organism capable of nitrogen fixation, as used herein, includes at least one cell, i.e. one or more cells. In plants, a “plant cell” refers to any cell of a plant, either taken directly from a seed or plant, or derived through culture from a cell or a tissue taken from a plant. A “plant cell” includes, for example, cells from undifferentiated tissue (e.g., callus), plant seeds, propagules, gametophytes, sporophytes, pollen, microspores, embryos, etc.

The transgenic plant or heterologous cell capable of nitrogen fixation further includes an expressible heterologous nucleotide sequence. The term “expressible,” “expressed,” and variations thereof refer to the ability of a cell to transcribe a nucleotide sequence to mRNA and translate the mRNA to synthesize a peptide or a polypeptide that provides a biological or biochemical function. For purposes of the present invention, this function includes Streptomyces thermoautotrophicus nitrogenase that catalyzes nitrogen fixation.

“Streptomyces thermoautotrophicus nitrogenase”, as applied to the UBT1 nitrogenase, refers to the complex of St1 plus St2 and, optionally, plus St3 (FIG. 1).

As used herein, “heterologous” refers to that which is foreign or non-native to a particular host, genome, gene or protein. Accordingly, a “heterologous nucleotide sequence” or “transgene” refers to a nucleotide sequence that originates from a species foreign to the host organism, or if the nucleotide sequence originates from the same species as the host, the nucleotide sequence is substantially modified from its native form in composition and/or genomic locus by deliberate genetic manipulation, which is present in the genome in a different location from which it is normally found, or which is found in a copy number in which it is not normally present. For instance, Streptomyces thermoautotrophicus nitrogenase is heterologous to any organism that is not Streptomyces thermoautotrophicus, such as plants, algae, other bacteria, animals, fungi, etc. Hence, “heterologous organism” or “heterologous cell”, for the purpose of expression or transformation with Streptomyces thermoautotrophicus nitrogenase, is any organism or cell which is not Streptomyces thermoautotrophicus. The term “nucleotide sequence” refers to a sequence of two or more nucleotides, such as RNA or DNA. A “heterologous protein” refers to a protein that is foreign or non-native to a host cell and is typically encoded by a heterologous nucleotide sequence.

Plants encompassed by the present invention include both monocots and dicots, C3 and C4 plants, agricultural, horticultural and ornamentals plants, oilseed plants, plants utilized for biomass, and algae. Non-limiting examples include plants such as corn, cotton, oil producing palms, various row crops, petunias, grasses and other plants.

Also encompassed by the present invention are parts of such plants including, for example, a protoplast, a cell, a tissue, an organ, a cutting, an explant, a reproductive tissue, a vegetative tissue and biomass. Such parts further include an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, a callus, a homogenate, propagation material, germplasm, cuttings, divisions and propagations.

The term “plant product” as used herein encompasses, but is not limited to, plants, plant parts, biomass and plant molecules that are customarily used for human food or animal feed. Furthermore, as used herein, plant products are not limited to edible products alone, but also include plants, plant parts, biomass and plant molecules such as, for example, pigments, fibers, cellulose, plant oils, lipids, fatty acids, sugars, medicinally active molecules, etc., that are useful in commercial products and processes such as lubricants, paints, pharmaceuticals, biofuels and other useful commercial products and applications known in the art.

The present invention also encompasses progeny, whether produced sexually or asexually, or through breeding techniques, of plants covered by the present invention or containing sequences disclosed herein. In regard to methods of propagating plants encompassed by the present invention, methods of propagation and reproduction of such plants are well known in the art, and include both sexual and asexual techniques. Asexual reproduction is the propagation of a plant to multiply the plant without the use of seeds to assure an exact genetic copy of the plant being reproduced. Any known method of asexual reproduction which renders a true genetic copy of the plant may be employed in the present invention. Acceptable modes of asexual reproduction include, but are not limited to, rooting cuttings, grafting, explants, budding, apomictic seeds, bulbs, divisions, slips, layering, rhizomes, runners, corms, tissue culture, nucellar embryos and any other conventional method of asexual propagation. All these and other methods of propagation and reproduction of plants are encompassed by the present invention.

In one aspect, plants capable of nitrogen fixation are rendered sterile and incapable of reproduction. Methods of introduction of sterility traits into plants are well known in the art and not detailed here for brevity (ex. Mitsuda et al, Plant Biotech J, 2006, 4:325-32).

Vectors

The term “vector” as used herein refers to a vehicle used for introduction of a nucleotide sequence into a host. A vector may be a plasmid, a cosmid, a phage, a transposon, a virus, or any other suitable vehicle. Preferably, the vector is a plasmid. A variety of vectors for transformation of eukaryotes and prokaryotes, plant, bacterial, cyanobacterial, archeal, protist, fungal, algal and animal cells are well known in the art. A vector may include operably linked regulatory sequences useful for expression of a gene product in a host, i.e. an expression vector, including but not limited to a promoter, a ribosomal binding site, a leader sequence, an intercistronic expression element (IEE), an internal ribosome entry site (IRES), an enhancer or a terminator sequences. When operably linked, such regulatory sequences perform their known and expected functions, facilitating gene or other nucleotide sequence expression. In one preferred embodiment, the vector is a vector for transforming a plastid as described below in one of the aspects of the invention.

In one embodiment, the heterologous nucleotide sequence or sequences can be placed in a single vector. For example, all Streptomyces thermoautotrophicus nitrogenase subunit genes (St3 being optional) can be placed in a single vector. In another embodiment heterologous nucleotide sequences, such as genes of Streptomyces thermoautotrophicus ntirogenase complex, can be placed separately in different vectors, which then can be used to transform a target cell. The heterologous nucleotide sequence can additionally include at least one gene encoding a cofactor for enhancing or modifying nitrogenase activity.

Vectors suitable for stable transformation of a plant cell are known in the art, and any suitable vector amongst the many known can be used to generate plants comprising Streptomyces thermoautotrophicus nitrogenase. Accordingly, the nitrogenase genes may be delivered into nuclear, or chloroplast (plastid), or mitochondrial genomes, or maintained as episomes. In one embodiment, for the transformation of nuclear host DNA, the vector is a binary vector (Lee and Gelvin, Plant Physiol, 2008, 146:325-332). A “binary vector” refers to a vector that includes a modified T-region from Ti plasmid, which allows replication in E. coli and in Agrobacterium cells, and usually includes selection marker genes. Examples of binary vectors are described later on. In another embodiment, the vector is a plastid or a chloroplast transformation vector (Lutz et al, Plant Physiol, 2007, 145:1201-1210, and Maliga, Trends Biotechnol, 2003, 21:20-28). Typically, a transgene in a chloroplast transformation vector is flanked by “homologous recombination sites,” which are DNA segments that are homologous to a region of the plastome. The “plastome” refers to the genome of a plastid. The homologous recombination sites enable site-specific integration of a transgene comprising expression cassette into the plastome by the process of homologous recombination. Chloroplast transformation vectors are described in detail later on.

Description of the aforementioned vectors is only exemplary and is not intended to limit the scope of the present invention. Other transformation vectors for transformation of plant, bacterial, fungal, algal or animal cells and methods for such transformations are well known in the art and for brevity are not described here in detail.

Promoters, Terminators and Other Genetic Elements

The heterologous nucleotide sequences or vectors described herein may include regulatory sequences useful for expression of a gene product in a host, such as a promoter, terminator or other genetic elements. The term “promoter” refers to a nucleotide sequence capable of controlling the expression of a coding sequence. A promoter drives expression of an operably linked nucleotide sequence. The term “operably linked” as used herein refers to linkage of a promoter to a nucleotide sequence such that the promoter mediates (drives) transcription of the nucleotide sequence. A “coding sequence” refers to a nucleotide sequence that encodes a specific amino acid sequence. A promoter is typically located upstream (5′) to the coding sequence, while a terminator is typically located downstream (3′) to the coding sequence. A variety of promoters are known in the art and may be used to facilitate expression of a gene. Examples of suitable promoters include constitutive promoters, plant tissue-specific promoters, fungal promoters, algal promoters, bacterial promoters, animal cell promoters, plant development (developmental stage) specific promoters, inducible promoters, circadian rhythm promoters, viral promoters, male or female germline-specific promoters, flower-specific promoters, chloroplast promoters, as well as other promoters well known in the art.

A “constitutive” promoter refers to a promoter that causes a gene to be expressed in all cell types at all times. An example of a constitutive plastid promoter is the chloroplast rrn (16S rRNA gene) promoter (SEQ ID NO: 11); an example of nuclear genomic constitutive plant promoters include the Cauliflower Mosaic Virus (CaMV) 35S promoter (SEQ ID NO: 12), which confers constitutive, high-level expression in most plant cells. Further examples of suitable constitutive promoters include the Rubisco small subunit (SSU) promoter, leguminB promoter, TR dual promoter, ubiquitin promoter, and Super promoter. Different heterologous nucleotide sequences or vectors may contain different promoters to prevent gene silencing when several transgenes are expressed in the same cell. Use of specific suppressors, such as P19 suppressor, to prevent transgene silencing is also well known in the art. Preferred constitutive promoters are strong promoters.

An “inducible” promoter refers to a promoter that is regulated in response to a stress or a stimulus, or is induced by a specific factor. Examples of inducible promoters include tetracycline repressor system, lac repressor system, copper-inducible system, salicylate-inducible system (such as the PR1a system), and alcohol-inducible system. Further examples include inducible promoters that are regulated in response to environmental, hormonal, chemical, and/or developmental stresses or stimuli. Such stresses or stimuli include heat (ex. tomato hsp70 promoter or hsp80 promoter), light, hormones (ex. abscisic acid), chemicals (ex. methyl jasmonate or salicylic acid), increased salinity, drought, pathogen (ex. promoter of the PRP1 gene), heavy metals (ex. heavy metal-inducible metallothionein I promoter and the promoter controlling expression of the tobacco gene cdiGRP) and wounds (ex. pinll promoter).

A “tissue-specific” promoter as used herein refers to a promoter that drives expression of an operably linked nucleotide sequence in a particular tissue. A tissue-specific promoter drives expression of a gene in one or more cell types in a specific organ (such as leaves, or seeds), specific tissues (such as embryo or cotyledon), or specific cell types (such as seed storage cells or leaf parenchyma). Examples include Gentiana triflora promoter for chalcone synthase (NCBI accession AB005484), a seed-specific promoter (such as β-conglycinin, napin promoter and phaseolin) and mature leaves-specific promoter (such as SAG promoter from Arabidopsis). Promoters responsible to the circadian rhythm cycle can also be used in the heterologous nucleotide sequence or vector. Such promoters include the native ELF3 promoter and the promoter from the chlorophyll a/b binding protein (CAB2 promoter). Further, a “developmental stage” promoter as used herein refers to a promoter that drives expression of an operably linked nucleotide sequence at a particular developmental stage of a plant. Examples of developmental stage promoters are known in the art.

Use of promoters of different strengths permits modulation of the level of expression of Streptomyces thermoautotrophicus nitrogenase, allowing modification (i.e. increase or decrease) in the level of the nitrogenase and accompanying nitrogen fixation activity appropriate for various types of plants (and other heterologous host cells and organisms) and environmental conditions, as desired or necessary. Manipulation of Streptomyces thermoautotrophicus nitrogenase gene dosage can also be used, alone or in combination with different strength promoters, to modulate nitrogen fixation activity to a desired level.

The heterologous nucleotide sequence or vector may also include a terminator or other genetic regulatory sequences. Terminator, or transcriptional terminator, is typically a genetic sequence that marks the end of a gene or an operon and promotes transcriptional termination. Examples of terminators include the chloroplast psbA terminator (SEQ ID NO: 13) and the eukaryotic Cauliflower Mosaic Virus (CaMV) 35S terminator (SEQ ID NO: 14). Additional genetic regulatory sequences may include, but are not limited, to elements such as internal ribosome entry sites (IRES), enhancers, leaders, Shine-Dalgarno sequences, PPR binding sequences and intercistronic expression elements (IEE) (Zhou et al, Plant J, 2007, 52(5): 961-972), as well as other regulatory elements known in the art.

Markers

A vector may include a nucleotide sequence for a selectable and/or screenable marker. A “selection marker” refers to a protein necessary for survival or growth of a transformed plant cell grown in a selective culture regimen. Typical selection markers include sequences that encode proteins, which confer resistance to selective agents, such as antibiotics, herbicides or other toxins. Examples of selection markers include genes for conferring resistance to antibiotics, such as spectinomycin, streptomycin, tetracycline, ampicillin, kanamycin, G418, neomycin, bleomycin, hygromycin, methotrexate, dicamba, glufosinate, or glyphosate. Various other selection markers confer a growth-related advantage to the transformed cells over the non-transformed cells. Examples include selection markers for β-glucuronidase (in conjunction with, for example, cytokinin glucuronide), mannose-6-phosphate isomerase (in conjunction with mannose), and UDP-galactose 4-epimerase (in conjunction with, for example, galactose).

Selection markers include those which confer resistance to spectinomycin (e.g. encoded by the resistance gene aadA, SEQ ID NO: 15), streptomycin, kanamycin, lincomycin, gentamycin, hygromycin, methotrexate, bleomycin, phleomycin, blasticidin, sulfonamide, phosphinothricin, chlorsulfuron, bromoxynil, glyphosate, 2,4-D, atrazine, 4-methyltryptophan, nitrate, S-aminoethyl-L-cysteine, lysine/threonine, aminoethyl-cysteine or betaine aldehyde. Preferably, the selection marker is functional when expressed either from plant nuclear, plastid or mitochondrial genomes. Selection markers functional in the heterologous cells and organisms are also useful. Especially preferred are the genes aadA (GeneBank NC_009838), nptlI (GeneBank FM177583), BADH (GeneBank AY050316) and aphA-6 (GeneBank X07753).

After a heterologous nucleotide sequence has been introduced into a host cell, it may be advantageous to remove or delete certain sequences from the targeted genome. For example, it may be advantageous to remove the selection marker gene that has been introduced into a genome if the selection marker is no longer required after the selection phase is complete. Methods for directed deletion of sequences are known in the art. For example, the nucleotide sequence encoding a selection marker preferably includes a homology-based excision element, such as Cre-lox and attB/attP recognition sequences, which allow removal of the selection marker genes using site-specific recombinases (Lutz et al, Nat. Protoc., 2006, 1900-10).

In one embodiment, the heterologous nucleotide sequence or vector includes a reporter gene. Reporter genes encode readily quantifiable proteins which, via their color or enzyme activity, allow assessment of transformation efficiency, selection of the transformed cells, site or time of expression or identification of transformed cells. Examples of reporter genes include green fluorescent protein (GFP), luciferase, β-galactosidase, β-glucuronidase (GUS), R-Locus gene product, β-Lactamase, xy1E gene product, alpha-amylase and tyrosinase.

Functional Elements

The heterologous nucleotide sequence or vector may also include functional elements, which influence the generation, multiplication, function, use, expression and other parameters of the heterologous nucleotide sequence or vector used within the scope of the present invention. Examples of functional elements include replication origins (ORI), which make possible amplification of the heterologous nucleotide sequence or vector according to the invention in, for example, E. coli or in plant plastids; multiple cloning sites (MCSs), which permit and facilitate the insertion of one or more nucleic acid sequences; homologous recombination sites, allowing stable recombination of transgenes into plastid genome; border sequences, which make possible Agrobacterium-mediated transfer of the heterologous nucleotide sequence or vector into plant cells for the transfer and integration into the plant genome, such as, for example, the right or left border of the T-DNA or the vir region. The heterologous nucleotide sequence or vector may optionally include RNA processing signals, e.g. introns, which may be positioned upstream or downstream or within a polypeptide-encoding sequence in the heterologous nucleotide sequence. Intron sequences are known in the art to aid in the expression of heterologous nucleotide sequences in plant cells.

Targeting Sequences

In another embodiment, the heterologous nucleotide sequence includes a targeting sequence, such as a plastid targeting sequence. A “plastid targeting sequence” as used herein refers to a nucleotide sequence that encodes a polypeptide which can direct a second polypeptide to an organelle (ex. a plastid) in a cell. Preferably, plastid targeting sequence is a chloroplast targeting sequence. Mitochondrial and other organelle and compartment targeting sequences are also contemplated by the present invention.

It is known in the art that non-chloroplast proteins may be targeted to the chloroplast or other organelles by use of protein fusions with a peptide encoded by a targeting sequence. For example, nitrogenase genes may be fused with a plastid targeting sequence. When the nitrogenase gene is expressed, the targeting sequence is included in the translated polypeptide. The targeting sequence then directs the polypeptide into a plastid such as a chloroplast.

In one embodiment, the chloroplast targeting sequence is linked to a 5′- or a 3′-end of the nitrogenase genes. Typically, the chloroplast targeting sequence encodes a polypeptide extension (called a chloroplast transit peptide (CTP) or transit peptide (TP)). The polypeptide extension is typically linked to the N-terminus of the heterologous peptide encoded by the heterologous nucleotide sequence. Examples of a chloroplast targeting sequences include a sequence that encodes Nicotiana tabacum ribulose bisphosphate carboxylase (Rubisco) small subunit (RbcS) transit peptide, Arabidopsis thaliana EPSPS chloroplast transit peptide, Petunia hybrida EPSPS chloroplast transit peptide and rice rbcS chloroplast targeting sequence. Further examples of a chloroplast targeting peptide include the small subunit (SSU) of ribulose-1,5,-biphosphate carboxylase and the light harvesting complex protein I and protein II. Those skilled in the art will recognize that various chimeric constructs can be made, if needed, that utilize the functionality of a particular CTP to import a given gene product into a chloroplast. Other CTPs that may be useful in practicing the present invention include PsRbcS derived CTPs (Pisum sativum Rubisco small subunit CTP), AtRbcS CTP (Arabidopsis thaliana Rubisco small subunit 1A CTP), AtShkG CTP (CTP2), AtShkGZm CTP (CTP2synthetic; codon optimized for monocot expression), PhShkG CTP (Petunia hybrida EPSPS; CTP4; codon optimized for monocot expression), TaWaxy CTP (Triticum aestivum granule-bound starch synthase CTPsynthetic, codon optimized for corn expression), OsWaxy CTP (Oryza sativa starch synthase CTP), NtRbcS CTP (Nicotiana tabacum ribulose 1,5-bisphosphate carboxylase small subunit chloroplast transit peptide), ZmAS CTP (Zea mays anthranilate synthase alpha 2 subunit gene CTP) and RgAS CTP (Ruta graveolens anthranilate synthase CTP). Other transit peptides that may be useful include maize cab-m7 signal sequence and the pea (Pisum sativum) glutathione reductase signal sequence. Additional examples of such targeting sequences may include: spinach lumazine synthase, Chlamydomonas ferredoxin, and Rubisco activase transit peptides, and other sequences known in the art.

Variants

The present invention further relates to variants of the nucleotide sequences described herein. Variants may occur naturally, such as a natural allelic variant or variant from a related species. Other variants include those produced by nucleotide substitutions, deletions or additions. The substitutions, deletions or additions may involve one or more nucleotides. These variants may be altered in coding regions, non-coding regions, or both. Alterations in the coding regions may produce conservative or non-conservative amino acid substitutions, deletions or additions. Preferably, the variant is a silent substitution, addition or deletion, which does not alter the properties and activities of the peptide encoded by the nucleotide sequence described herein. Conservative substitutions are also preferred.

Further embodiments of the invention include variant nucleotide or amino acid sequences comprising a sequence having at least 80% sequence identity or homology, and more preferably at least 90%, 91%, 92%, 93%, 94%, 95%, 96%, 97%, 98%, or 99% sequence identity or homology, to the nucleotide or amino acid sequence of the nitrogenase or one of the nitrogenase functional domains or subunits. For example, a variant nucleotide sequence that is at least 95% sequence identical to a nitrogenase sequence is identical to the latter sequence except that the variant nucleotide sequence may include up to five point mutations per each 100 nucleotides of the nitrogenase sequence. In other words, to obtain a variant nucleotide sequence that is at least 95% identical to a nitrogenase nucleotide sequence, up to 5% of the nucleotides in the claimed sequence may be deleted or substituted with another nucleotide, or a number of nucleotides up to 5% of the total nucleotides may be inserted into the nitrogenase sequence.

These mutations of the nitrogenase or nitrogenase functional domain sequences or subunits may occur at the 5′ or 3′ terminal positions of the sequence, or anywhere between those terminal positions, interspersed either individually among nucleotides in the nitrogenase sequence or in one or more contiguous groups within the nitrogenase sequence. The term “sufficiently identical” as used herein refers to a first nucleotide sequence that contains a sufficient or minimum number of identical or equivalent nucleotides to a second nucleotide sequence, such that the first and second nucleotide sequences share common structural domains or motifs and/or a common functional activity. For example, nucleotide sequences that share common structural domains having at least 80%, 85%, 90%, 91%, 92%, 93%, 94%, 95%, 96%, 97%, 98%, 99%, or more identity across the sequences, and share a common functional activity are defined herein as sufficiently identical.

A “nitrogenase protein”, or “subunit thereof”, or any other protein or peptide presently disclosed and utilized in any of the methods and plants, or other organisms disclosed herein, refers to a protein or peptide exhibiting enzymatic or functional activity similar or identical to the enzymatic or functional activity of the specifically named protein or peptide. Enzymatic or functional activities of the nitrogenase proteins and peptides disclosed herein are described in Ribbe et al, J Biol Chem, 1997, 272(42):26627-33. “Similar” enzymatic/functional activity of a protein or peptide can be in the range from about 50% to about 200%, or more, of the enzymatic or functional activity of the specifically named protein or peptide when equal amounts of both proteins or peptides are assayed, tested or expressed as described in Ribbe et al., supra, or below under identical conditions and can therefore be satisfactory substituted for the specifically named proteins or peptides in present methods and transgenic plants, algae and other organisms encompassed herein to catalyze atmospheric nitrogen fixation.

To determine percent identity of two nucleic acid sequences, the sequences are aligned for optimal comparison purposes (e.g., gaps can be introduced in one or both of a first and second nucleotide sequence for optimal alignment). For example, when aligning a first sequence to a second sequence having 10 nucleotides, at least 70%, preferably at least 80%, more preferably at least 90% of the 10 nucleotides between the first and second sequences are aligned. When a position in the first sequence is occupied by the same nucleotide as the corresponding position in the second sequence, then the molecules are identical at that position. The percent identity between the two sequences is a function of the number of identical positions shared by the sequences, taking into account the number of gaps, the length of the sequences, and the length of each gap that need to be introduced for optimal alignment of the two sequences. Computer software and algorithms known in the art may be used to determine percent identity between two given sequences.

As used herein, the phrase “sequence identity” means the percentage of identical nucleotide or amino acids residues at corresponding positions in two or more sequences when said sequences are aligned to maximize sequence matching, i.e. talking into account gaps and insertions. Identity can be readily calculated by known methods, including but not limited to those described in: Biocomputing: Informatics and Genome Projects, Smith D. W., ed., Academic Press, New York, 1993; Sequence Analysis in Molecular Biology, von Heinje G., Academic Press, 1987; Computational Molecular Biology, Lesk A. M., ed., Oxford University Press, New York, 1988; Computer Analysis of Sequence Data, Part I, Griffin A. M. and Griffin H. G., eds., Humana Press, New Jersey, 1994; Sequence Analysis Primer, Gribskov M. and Devereux J., eds., M Stockton Press, New York, 1991; and Carillo H. and Lipman D., SIAM J. Applied Math., 48:1073 (1988). Methods to determine identity can also be found in publicly available computer programs.

Optimal alignment of sequences for comparison can be conducted, for example, by the local homology algorithm of Smith and Waterman, by the homology alignment algorithms, by search for similarity method or, by computerized implementations of these algorithms (BLAST, GAP, BESTFIT, PASTA and TEASTA in the GCG Wisconsin Package, available from Accelrys Inc., San Diego, Calif., USA), or other algorithms and methods known in the art. One example of an algorithm that is suitable for determining percent sequence identity and sequence similarity is the BLAST algorithm, which is available in public access at NCBI/NIH and originally described in Altschul S. et al., NCBI NLM NIH Bethesda, Md., and Altschul S. F. et al., J. Mol. Biol. 215: 403-410 (1990). Software for performing BLAST analyses is publically available through the National Center for Biotechnology Information (NCBI).

Enhanced and/or Modified Nitrogenase Activity

In one aspect, nitrogen fixation levels can be increased or decreased by an increase or decrease in activity or level of enzymes involved in the nitrogen fixation reaction. For example, a nitrogenase can be expressed under a strong promoter, thereby allowing increase in concentration of the nitrogenase and nitrogenase-related proteins within a given cell and thus higher N₂ fixation levels, as compared to a cell with a weaker promoter. Use of a strong constitutive promoter is expected to permit transformed plants to express high levels of the nitrogenase, and fix nitrogen, over prolonged periods of time. Use of inducible, tissue-specific and developmental stage promoters permits transformed plants to express the nitrogenase, and fix nitrogen, during development when and where in the plant specifically needed or desired.

Alternatively, nitrogenase levels, and concomitant nitrogen fixation, can be increased by expressing multiple copies of the enzyme complex, including expression from different organelles, such as plastids, nuclei, mitochondria, or from vector(s) maintained as an episome, or various combinations thereof, simultaneously. Once again, use of a constitutive promoter, an inducible promoter, a tissue-specific promoter, or a developmental stage promoter for such expression can be employed to controllably modulate nitrogen fixation to achieve desired levels as well as stage of development and location within plants.

Thus, nitrogenase levels and concomitant levels of N₂ fixation can be controllably modulated to optimize plant or other host cell, or organism, growth by use of promoters of different strength, tissue specificity, inducibility, developmental stage specificity and duration of transcriptional activity. Nitrogenase levels and levels of N₂ fixation can also be modulated by the extent of gene dosage (copy number) in any single intracellular genome, or combination of genomes. In addition to employing any of these strategies alone, combinations of any of the forgoing methods can be used to optimize N₂ fixation compared to that in control or wild type plants or host cells or organisms, or in prototype versions of transgenic plants or host cells or organisms.

The term “enhance”, “enhanced”, “increase”, “increased”, “decrease”, “decreased”, “modify”, “modified”, “optimize”, “optimized”, “modulate” or “modulated”, and the like, refer to a statistically significant increase or decrease in a parameter or value herein. For the avoidance of doubt, these terms refer to about a 5% increase in a given parameter or value, about 10% increase, about a 15% increase, about a 20% increase, about a 25% increase, about a 30% increase, about a 35% increase, about a 40% increase, about a 45% increase, about a 50% increase, about a 55% increase, about a 60% increase, about a 65% increase, about a 70% increase, about a 75% increase, about an 80% increase, about an 85% increase, about a 90% increase, about a 95% increase, about a 100% increase, or more, over the baseline value, or comparative parameter value in a prototype trangenic organism, and similarly for decreases, modifications, optimizations, etc. These terms also encompass continuous ranges consisting of any lower indicated value or any higher indicated value, including ranges of any pints in between, for example, from about 5% to about 50%, etc. Any ranges that can be formed by any of the values or data presented herein represent further embodiments of the present invention.

In another aspect, use of a variety of translational, transcriptional and other enhancing elements (e.g., IEE sites, enhancers, etc.), as well as co-expression of additional proteins allowing to stabilize, enhance or improve nitrogenase activity, can be used to enhance or modify plant nitrogen fixation.

In yet another aspect, methods to modify and increase nitrogenase and/or other related enzymes activities may include directed evolution, codon optimization, protein engineering, rational design and other similar methods well known in the art. These and other methods are known to significantly improve enzyme activity, selectivity, stability and other parameters, as compared to an identical naturally occurring enzyme that has not undergone these improvement processes (ex. Cobb et al, Curr Opin Chem Biol, 2012, 16(3-4):285-91).

Cofactors

As used herein, the term “cofactor” refers to an organic molecule, an inorganic molecule, a peptide, a protein, or a nucleotide required for or enhancing an enzyme activity. Examples of co-factors useful for enhancing nitrogenase activity may include manganese, molybdenum or iron ions, polypeptides, CoxL, CoxM and CoxS proteins, and other molecules.

Nitrogenase Crosstalk

In one aspect, the invention relates to nitrogenase crosstalk, where a first heterologous sequence includes gene or genes coding for nitrogenase, wherein said first heterologous nucleotide sequence is operably linked to a first promoter. The nitrogenase crosstalk further comprises a vector having a second heterologous nucleotide sequence operably linked to a second promoter.

For example, the promoter for the first heterologous nucleotide sequence, the nitrogenase, is inducible by the second heterologous nucleotide sequence, a transcription factor. An exemplary inducible promoter is T7 promoter, which is activated by T7 RNA polymerase. Yet another exemplary inducible promoter is the UAS promoter, inducible by Gal4 binding domain fused to a VP16 transcriptional activator. There are other multiple examples of inducible promoter/activator pairs known in the art. In one embodiment, the promoter for the second heterologous nucleotide sequence is a tissue-specific promoter. The second heterologous nucleotide sequence, by way of example only, is a T7 RNA polymerase. Accordingly, when the tissue specific promoter is activated, the gene for the T7 RNA polymerase will be transcribed and activate the nitrogenase gene driven by the inducible T7 promoter. Thus, nitrogenase activation is indirect and occurs via nitrogenase crosstalk.

Methods for Producing Plants, Plant Cells and Heterologous Cells Comprising a Nitrogenase

In one aspect, the invention describes creation of genetically modified plants capable of fixing nitrogen on their own. Genetically modified plants capable of nitrogen fixation further include an expressible heterologous nucleotide sequence. The term “expressible,” “expressed,” and variations thereof refer to the ability of a cell to transcribe a nucleotide sequence to mRNA and translate the mRNA to synthesize a polypeptide that provides a biological or biochemical function. Preferably, the cell is a plant cell. As used herein, “heterologous” refers to that which is foreign or non-native to a particular host or genome. Accordingly, a “heterologous nucleotide sequence” or “transgene” refers to a nucleotide sequence that originates from a species foreign to the host organism, or if the nucleotide sequence originates from the same species as the host, the nucleotide sequence is substantially modified from its native form in composition and/or genomic locus by deliberate genetic manipulation, or is present in a location in a genome in which it is not normally found, or is present in more than the usual number of copies. The term “nucleotide sequence” refers to a sequence of two or more nucleotides, such as RNA or DNA. A “heterologous protein” refers to a protein that is foreign or non-native to a host cell and is typically encoded by a heterologous nucleotide sequence.

The term “transfecting” or “transforming” refers to introducing a nucleotide sequence into a host cell or into a plastid of the cell. The nucleotide sequence that is being introduced to the host cell nuclear genome, or plastid genome, or mitochondrial genome, or maintained as an episome, or other location within the cell, may include a heterologous nucleotide sequence or a vector as described above. Transfection of the heterologous nucleotide sequences is achieved by methods known to a skilled artisan. Any method that permits the introduction of a nucleotide sequence into a plant cell is suitable. Examples of such methods include transformation of chemically competent cells, microinjection, electroporation, biolistic bombardment with DNA-coated microparticles (“gene gun” method), permeabilizing a cell with polyethylene glycol, silicon whiskers, fusion with other DNA-comprising units such as minicells, hybridomas, hybrid cells or liposomes. Preferred methods include, for example, biolistic gene delivery and Agrobacterium mediated transformation.

Similarly, a variety of vectors and methods are known in the art to introduce heterologous sequences into bacterial (including cyanobacteria), fungal, algal or animal cells, thus resulting in transgenic organisms expressing the heterologous sequences. These methods are well known to a skilled artisan and can be used to stably or transiently express nitrogenase in a heterologous system. For the sake of brevity, only plant transformation methods will be described in detail and all other methods known in the art are incorporated herein by reference in their entirety.

Methods for regulating biological processes are known in the art, and may include various constitutive or inducible promoters, enhancers or silencing sequences and other means. These methods can be used to up- or down-regulate nitrogen fixation capacity of cells expressing Streptomyces thermoautotrophicus nitrogenase.

Plant Nuclear Genome Transformation

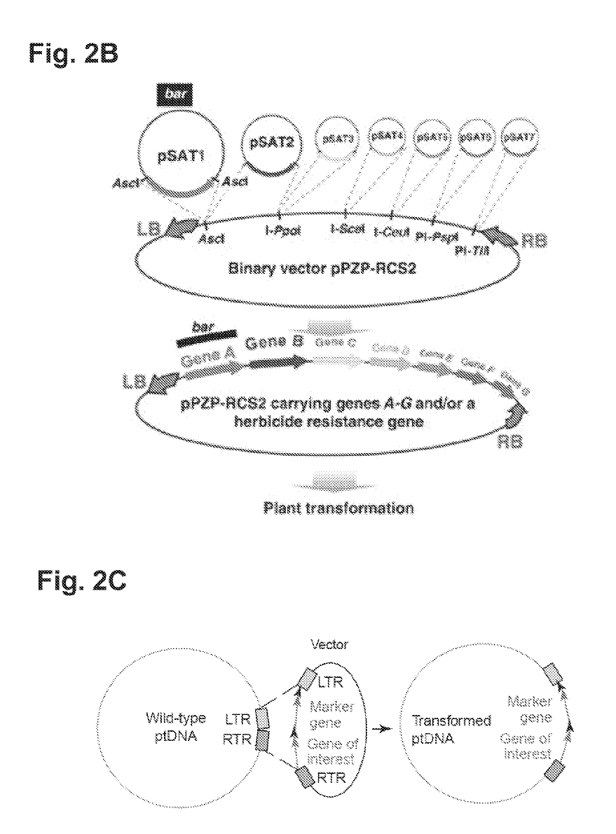

In one aspect, Agrobacterium is an effective tool for transforming plant nuclear genomes. The process of plant genetic transformation by Agrobacterium has been extensively characterized and is well known in the art. Agrobacterium T-DNA is used as a vehicle for delivering the gene or genes of interest (GOI) into the host genome. Initially, this technology was based on cloning GOIs directly into the T-DNA region of the Ti plasmid. However, this approach was technically challenging due to the large size and low copy number of Ti plasmids, leading to difficulties in plasmid isolation and manipulation. It was replaced by binary vector systems (Lee and Gelvin, Plant Physiol, 2008, 146:325-332) composed of two plasmids: (i) the helper plasmid—the Agrobacterium Ti plasmid carrying the vir genes, but lacking a functional T-DNA segment, and (ii) the binary vector constructed on a DNA backbone derived from commonly used E. coli cloning vectors and carrying the GOI flanked by 25 bp-long right and left T-DNA border sequences (RB and LB) (FIG. 2A). The binary system is based on the principle that T-DNA and the molecular machinery required for its transfer, encoded by the vir genes, function in trans and thus can be separated into two different plasmids within the same Agrobacterium cell. Whereas genetic manipulations are performed on the binary plasmid in E. coli using standard cloning procedures, the helper plasmid is usually maintained within Agrobacterium cells. When construction of the binary vector is completed, it is introduced into Agrobacterium carrying the helper plasmid to reconstitute the transformation-competent binary system. Numerous binary vectors have been developed and are known in the art, but most of them are limited to carrying a single selection marker and one or two GOIs. This is mainly dictated by the fact that each monocistronically expressed eukaryotic ORF must contain its own regulatory sequences (e.g. promoters and terminators), thus significantly increasing binary vector size and complicating cloning procedures. Examples of binary vector systems that address this limitation and allow straightforward incorporation of multiple GOIs into a plant genome on the same T-DNA are well known in the art (for example see FIG. 2B and Tzfira et al, Plant Mol Biol, 2005, 57(4):503-16). Yet, nitrogenase gene or genes may be incorporated into plant nuclear genome using single vector, or multiple binary vectors, to allow expression of fully functional enzyme within a plant cell.

Agrobacterium armed with an appropriate binary vector is used to generate transgenic plants. Arabidopsis thaliana and Nicotiana tabacum are among the most commonly used model organisms for both nuclear and chloroplast genetic transformation due to well-developed and efficient protocols for DNA delivery and recovery of transformants. Stable nuclear transformation of Arabidopsis germline cells can be rapidly achieved by the floral-dip method, where flowers are dipped in liquid Agrobacterium culture. Seeds from the dipped plants are collected and geminated on herbicide-supplemented selective media, which allows only transgenic plants expressing the selection marker gene contained in the T-DNA to survive. For tobacco, leaf disk inoculation, is a commonly used method to produce transgenic plants. Leaf disks are submerged in liquid Agrobacterium culture and then transferred to a callus inductive medium that has been supplemented with appropriate herbicide and plant hormones. The herbicide resistance gene contained in the T-DNA ensures that only the transformed cells survive, and the plant hormones promote regeneration of plants from the surviving cells. Another highly preferred method is plant transformation using biolistic DNA delivery, and additional methods of transformation by electroporation and polyethylene glycol (PEG)-mediated DNA delivery are known in the art. Any suitable known method known can be used to produce plants comprising a nitrogenase. Particularly preferred are commercial plants including corn, cotton, various row crops, ornamental plants, as well as any other agriculturally or commercially important plant.

Plastid Genetic Engineering

A plant cell typically contains a “plastid,” which refers to an organelle with its own genetic machinery in a plant cell. Examples of plastids include chloroplasts, chromoplasts, etioplasts, gerontoplasts, leucoplasts, proplastids, amyloplasts, elaioplasts, etc. The prokaryotic nature of chloroplast DNA integration and gene expression mechanisms necessitates chloroplast transformation vectors to be constructed differently than binary vectors. Unlike Agrobacterium-mediated genetic transformation, where T-DNA integrates randomly, chloroplasts support homologous recombination allowing targeted integration of the transgene. Therefore, in chloroplast transformation vectors, the gene or genes of interest (GOI) can be flanked by two sequences homologous to the selected integration site within the genome, also known as LTR and RTR (FIG. 2C, Maliga, TRENDS in Biotech, 2003, 21(1):20-28). Generally, the integration site should be chosen to avoid insertions within essential genome areas that might negatively impact plant development and viability. To date, the most commonly used integration sites are the intergenic regions between the tRNA-Ile (TrnI) and tRNA-Ala (TrnA) genes and between the tRNA-Val (TrnV) and rps12/7 operon. Whereas transgenes integrated into the TmV-rps12 site must be equipped with their own promoter sequences, the TrnI-TrnA integration site is adjacent to the 16S rRNA promoter, which drives read-through transcription through this integration site, potentially allowing the use of promoterless gene or genes of interest (GOIs).

Unlike monocistronically expressed GOIs integrated into the nuclear genome, polycistronic gene expression in chloroplasts allows the use of only one promoter and terminator sequence for multiple ORFs organized in an operon-like gene group. In this arrangement, each ORF requires a separate ribosome binding site (RBS) for translation initiation. Numerous bacterial and plastid RBSs (including Shine-Dalgarno [AGGAGG] sequence), are known in the art. The polycistronic nature of chloroplast gene expression also permits easy cloning of multiple transgenes—particularly those derived from an existing bacterial operon—into chloroplast transformation vectors and their integration into the chloroplast genome in a single transformation step. Alternatively, nitrogenase genes can be arranged as an artificial operon for expression.

In one embodiment, the preferred method of chloroplast transformation is biolistic DNA delivery. The biolistic chloroplast transformation methodology is well known in the art (Verma et al, Nat Prot, 2008, 3:739-758 and Lutz et al, Nat Prot, 2006, 1900-10). Briefly, tobacco leaf explants are bombarded with vector-coated gold microparticles and transplastomic plants are regenerated on a medium containing appropriate hormones and selection agents. Not all plant species can be transformed and regenerated using their leaf explants. Transplastomic plants of agronomically important species, such as cotton and soybean, are produced via somatic embryogenesis. Other methods for plastid transformation known in the art can also be used.

In one embodiment, nitrogenase genes from Streptomyces thermoautotrophicus can be expressed either as monocistronic mRNAs or as an operon (a polycistronic mRNA) from the plastidal genome, by integration via single or multiple vectors. The term “operon” refers to a nucleotide sequence which codes for a group of genes transcribed together. The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression. Some genes can be transcribed into mRNA and translated into polypeptides (structural genes); other genes can be transcribed into RNA (e.g. rRNA, tRNA), and other types of genes function as regulators of expression (regulator genes). Streptomyces thermoautotrophicus nitrogenase genes required for nitrogen fixation can be assembled in an operon and may include genes encoding for L, M and S subunits of component St1 (also referred as sdnL, sdnM and sdnS, respectively), and gene encoding for D subunit of component St2 (also referred as sdnO gene). The operon may optionally include, but is not limited to, genes CoxL, CoxM and CoxS of the component St3, or as required to restore a fully functional nitrogenase system within a specific plant cell.

The nitrogenase sequence (or nitrogenase subunits amino acid sequences) can alternatively be expressed from other nucleic sequences within a plant cell or a heterologous cell including nuclear or mitochondrial genomes (example of mitochondrial genome transformation can be found in Remade et al, PNAS USA, 2006, 103:4771-4776), episomes or other nucleic acid sequences (plasmid, viral, etc) which are known in the art.

### EXAMPLES

The following presented examples are meant to be illustrative and not limiting of the practice or products of the present invention. The examples below show introduction of Streptomyces thermoautotrophicus nitrogenase genes into chloroplasts of a commonly known plant model organism, Nicotiana tabacum, as well as other plant species, and generation of plants comprising nitrogenase and capable of nitrogen fixation. Methods for generation of additional species of transgenic plants, or heterologous transgenic organisms, are well known in the art and are not described here in detail for conciseness.

### Example 1

### Construction of Plant Transformation Vectors

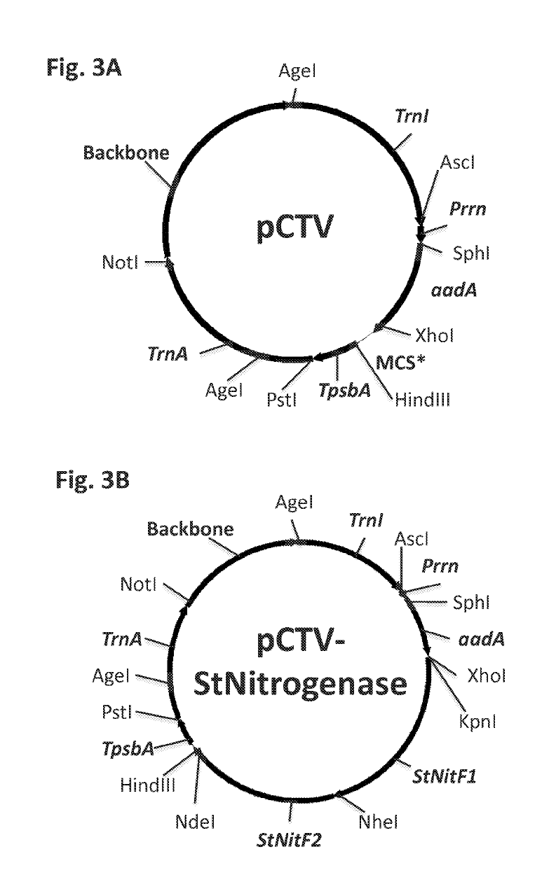

An exemplary chloroplast transformation vector pCTV (Chloroplast Transformation Vector) for nitrogenase expression can be constructed on the basis of essentially any standard cloning vector. For example, a standard cloning vector pUC19 (GeneBank #L09137 and SEQ ID NO: 16) can be used. Any other suitable cloning vector can be used to construct a nitrogenase bearing vector. In this example, pUC19 multiple cloning site (MCS) was replaced by a sequence containing an expanded number of restriction enzyme recognition sites to allow cloning of multiple genetic elements. The new expanded MCS contained the following restriction sites: AgeI-AscI-SphI-BgIII-XhoI-EcoRI-SaclI-KpnI-EcoRV-NheI-SpeI-SalI-SacI-NdeI-BamHI-StuI-KasI-PacI-FseI-SwaI-HindIII-PstI/SbfI-NotI-SmaI. The chloroplast Prm promoter (SEQ ID NO: 11) was cloned as an AscI/SphI PCR fragment; other promoters, such as the chloroplast psbA promoter (GeneBank DQ463359, SEQ ID NO: 17), can be used. The chloroplast psbA terminator sequence (SEQ ID NO: 13) was cloned as a HindIII/PstI fragment; other suitable terminators known in the art can be used. The spectinomycin resistance gene aadA (SEQ ID NO: 15), driven by Shine-Dalgarno (AGGAGG or AGGAGGT) leader sequence was cloned into pCTV as a SphI/XhoI PCR fragment; other suitable selection markers are known in the art and can be used. To make a pCTV vector suitable for integration into the chloroplast genome, homologous recombination (HR) sequences were cloned to flank the nitrogenase expression cassette. In one embodiment, the integration site is the TrnI/TmA locus within tobacco chloroplast genome; other integration sites can be selected. The TrnI HR (SEQ ID NO: 18) was cloned as an AgeI/AscI PCR fragment, followed by TrnA HR (SEQ ID NO: 19) which was cloned as a PstI/NotI PCR fragment. An exemplary pCTV sequence and map are shown in SEQ ID NO: 20 and FIG. 3A, respectively. A nitrogenase gene or genes, containing the desired genetic features (e.g. ribosome binding sites, etc.), can be further cloned into the multiple cloning site between the aadA gene and the psbA terminator (marked as MCS* in FIG. 3A).

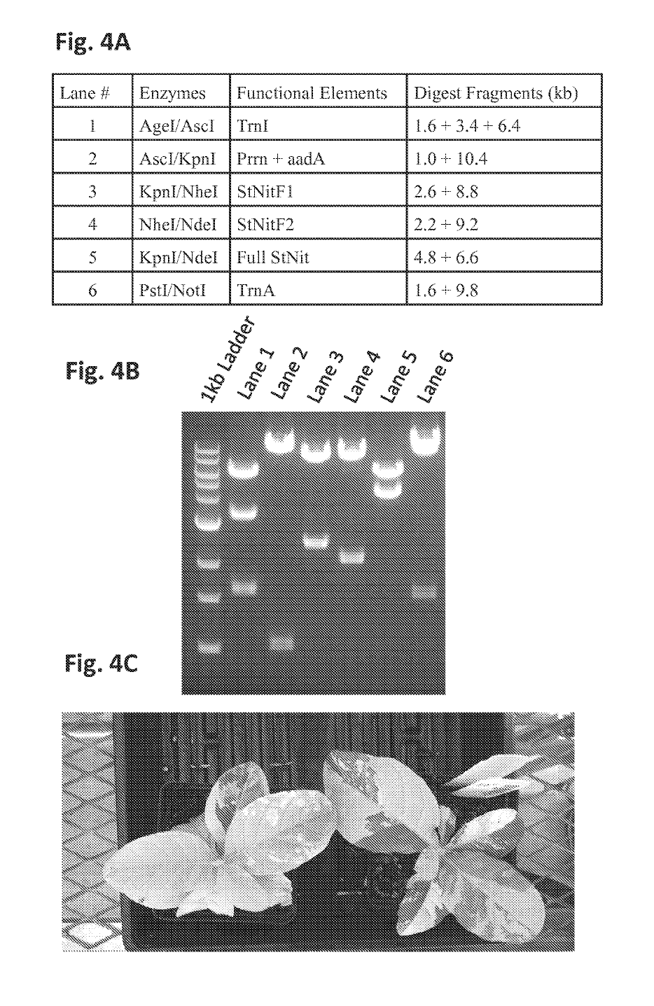

In one embodiment, the nitrogenase compex from Streptomyces thermoautotrophicus is cloned into pCTV for expression in the form of synthetic operon containing genes: sdnL-sdnS-sdnM-sdnO, encoding for Streptomyces thermoautotrophicus nitrogenase proteins (SEQ ID NOs: 21-24). It should be noted, that genes in this operon can optionally be positioned in different order from that described herein. The cloning of the nitrogenase operon was performed, for technical simplicity, as two separate segments. The first segment comprising sdnL gene, optimized for expression in chloroplasts (designated as StNitF1; SEQ ID NO: 25), was cloned as a KpnI/NheI fragment. The second segment comprising sdnS-sdnM-sdnO genes, optimized for expression in chloroplasts (designated as StNitF2; SEQ ID NO: 26), was cloned as an NheI/NdeI fragment. Genes of the operon are driven by Shine-Dalgarno sequences and separated by intercistronic expression elements, resulting in pCTV-StNitrogenase vector suitable for chloroplast transformation (SEQ ID NO: 27 and FIG. 3B). Optionally, the genes can be further regulated by a variety of other elements, for example repressors or enhancers known in the art (e.g. LacO repressor, T7 promoter, PPR binding sequences, a variety of leader sequences, protein stabilizing elements, etc.) to enhance or reduce nitrogenase expression or activity. Reporter genes (ex. GUS or GFP) can also be included in the expression cassette to track gene expression or to identify transgenic plants. Exemplary digest, including table showing expected fragments and the actual digest of the prepared pCTV-StNitrogenase vector resolved on an ethidium bromide stained 1% agarose gel, are shown in FIGS. 4A and 4B, respectively.

In addition to pCTV-StNitrogenase, a number of supplementary vectors have been prepared on the basis of pCTV. First, a vector for preparation of negative control N. tabacum plants have been generated by cloning a reporter beta-glucuronidase (GUS) gene downstream of aadA in pCTV vector. The resulting vector has been designated as pCTV-GUS and utilized to produce transplastomic plants that served as additional negative experimental controls, side by side with wild type tobacco plants. Yet another vector, carrying GUS positioned downstream of Streptomyces thermoautotrophicus nitrogenase in pCTV-StNitrogenase, vector has been constructed to serve as a supplementary experimental tool and was designated as pCTV-StNitrogenase-GUS. All results for plants generated using pCTV-StNitrogenase-GUS were similar to plants generated using pCTV-StNitrogenase and therefore are not detailed here for brevity.

### Example 2

### Generation of Plants Comprising Streptomyces thermoautotrophicus Nitrogenase

Plants comprising Streptomyces thermoautotrophicus nitrogenase (experimental plants), as well as plants comprising GUS (control plants), were produced using methods well known in the art (Verma et al, Nat Prot, 2008, 3:739-758 and Lutz et al, Nat Prot, 2006, 1900-10). Briefly, 0.6 micron gold particles (BioRad) coated with the vector DNA were bombarded into leaves of aseptically grown 4-6 weeks old Nicotiana tabacum plants (cv. Petit Havana) using PDS-1000/He Biolistic Particle Delivery System (system settings: bombardment He pressure approx. 250 psi above rapture disk pressure, [rapture disks of 1,100 psi are typically used]; distance from the top of the chamber 9cm [third slot], chamber vacuum pressure 28 in Hg). The bombarded leaves were incubated at 25-26° C. in the dark for 2-3 days and dissected to 5×5 mm squares, which were placed in deep Petri dishes containing 50 ml of RMOP medium (RMOP per liter: MS salts [Caisson, according to manufacturer's instructions]; 100 mg myo-inositol; 1 mg thiamine HCl; 1 mg 6-benzylamino purine; 0.1 mg 1-naphthaleneacetic acid; 30 gr sucrose; 7-8 g phytoblend [Caisson], pH=5.8 adjusted with KOH, and supplemented with 500 μg/ml of spectinomycin [Sigma]). The Petri dishes were sealed with parafilm and cultivated under cool-white fluorescent lamps (1,900-2,000 lux) with 16 h light/8 h dark cycle at 26° C. Transgenic plants appeared within 4-8 weeks post bombardment. The plants were transferred and further aseptically maintained in magenta boxes on MSO medium (MSO per liter: MS salts [Caisson, according to manufacturer's instructions]; 30 gr sucrose; 7-8 g phytoblend [Caisson], pH=5.8 adjusted with KOH, supplemented with MS Vitamins [Phytotechnology Laboratories, according to manufacturer instructions]), and further grown under cool-white fluorescent lamps (1,900-2,000 lux) with 16 h light/8 h dark cycle at 26° C. Typically, pCTV-StNitrogenase plants regenerated after bombardment have shown chimeric/heteroplastomic phenotype (FIG. 4C) and required additional 2-3 regeneration rounds on RMOP media, as known in the art (Lutz et al, Nat Prot, 2006, 1900-10), to produce non-chimeric plants.

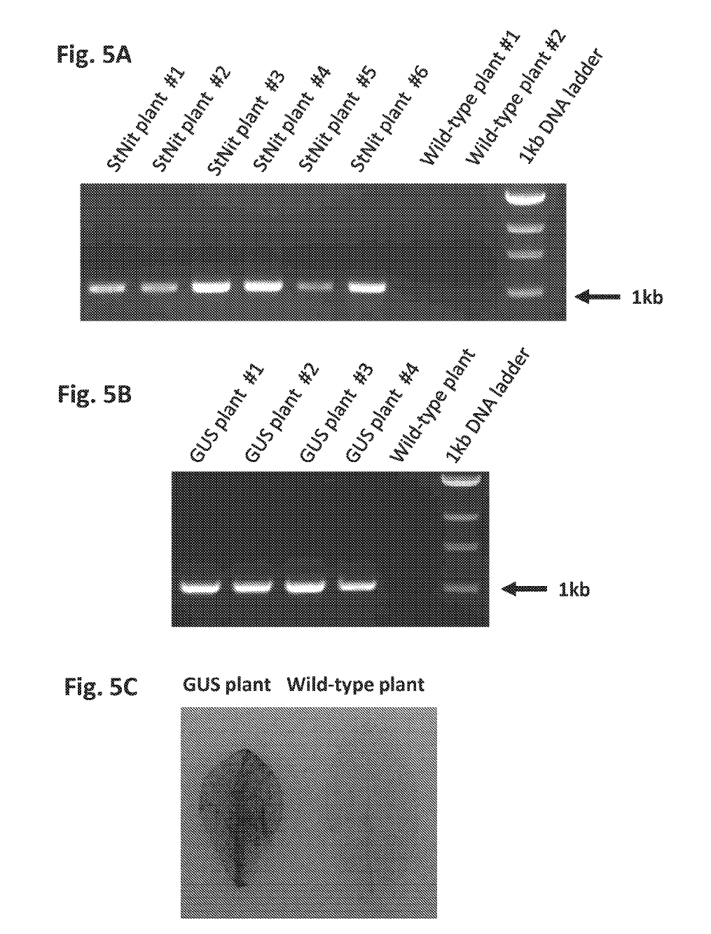

All plants surviving spectinomycin selection regimen have been further validated using PCR and histochemical staining (for GUS expressing plants). Pairs of primers have been designed to specifically and accurately amplify DNA sequences integrated into the plant genome. Primer P1 (SEQ ID NO: 28), directed upstream from the TpsbA terminator, and primer P2 (SEQ ID NO: 29), directed downstream from Streptomyces thermoautotrophicus sequence, have been used to confirm nitrogenase comprising plants. Primers P1 and P3 (SEQ ID NO: 30), directed downstream from the GUS sequence, were used to identify GUS comprising plants. Both pairs of primers, P1+P2 and P1+P3, were designed to produce highly specific diagnostic bands of approx. 1 kb size when used to amplify pCTV-StNitrogenase and pCTV-GUS templates, respectively. DNA from leaves of the transformed aseptically grown plants was prepared using methods known in the art and used as a template in a PCR reaction driven by Taq polymerase (Takara); reaction products were resolved on 1% agarose gel. About half a dozen plants of each type have been positively identified, with exemplary results shown in FIG. 5A for Streptomyces thermoautotrophicus nitrogenase comprising plants (“StNit plants”) and in FIG. 5B for GUS comprising plants (“GUS plants”). Wild-type tobacco DNA was used as negative control, demonstrating high specificity and precision of the PCR reaction.

In addition, GUS carrying plants have been tested and confirmed for GUS expression using X-Gluc and methods well known in the art. Briefly, leaves or leaf parts from aseptically grown GUS expressing plants have been excised and incubated with 0.5 mg/ml of X-Gluc in phosphate buffer for 5-6 hours at 37° C., followed by overnight incubation with 75% EtOH solution for removal of chlorophyll. FIG. 5C demonstrates exemplary staining of leaves of wild-type and GUS-comprising plants, showing strong GUS expression in GUS-comprising plants and lack thereof in wild-type control plants.

### Example 3

### Plants Comprising Streptomyces thermoautotrophicus Nitrogenase Show Phenotype Highly Resistant to Nitrogen Deficiency and are Capable of Direct Nitrogen Fixation from the Atmosphere

Plants carrying Streptomyces thermoautotrophicus nitrogenase in their genome and produced as described in Examples 1 and 2 (“StNitrogenase plants”) showed phenotype highly resistant to nitrogen deficiency. Experimental plants, generated using either pCTV-StNitrogenase or pCTV-StNitrogenase-GUS vectors (both demonstrating similar results), have been compared to control plants, either wild type or plants generated using pCTV-GUS (both demonstrating similar results). Apical cuttings of experimental and control plants have been transferred to nitrogen deficient MSO medium (N-free MSO), comprising

per liter: N-free MS salts (MS Modified Basal Salt: w/o Nitrogen, Phytotechnology Laboratories, cat #M531, according to manufacturer's instructions); MS vitamins (Phytotechnology Labs); 30 gr sucrose; 7-8 g plant tissue culture agar (Sigma, A7921), pH=5.8 adjusted with KOH. Magenta boxes containing aseptically grown plants have been opened in a flow hood and aerated for approx. 5mins every 2-3 days to allow air exchange and atmospheric nitrogen access.

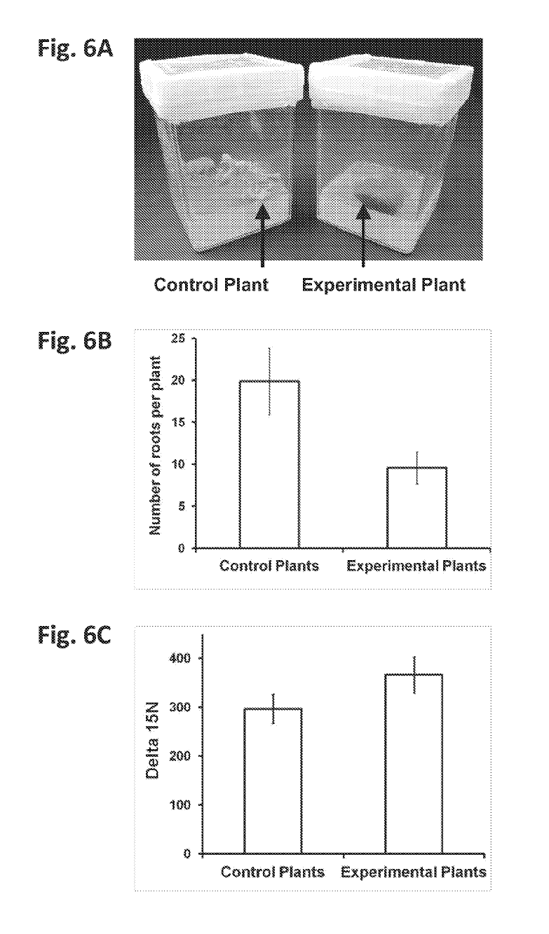

Within 7-10 days, control plants started showing clear signs of nitrogen deficiency, while experimental plants did not. First, typical symptoms of tobacco nitrogen deficiency in foliage started appearing in the control plants manifested as “fired” appearance of bottom leaves (Tucker, NCDA&CS, 1999, pp. 1-9), which started browning and curling at the tips, further spreading towards the leaf base. The experimental StNitrogenase plants, however, did not show these symptoms at this stage (FIG. 6A). Second, it is known that nitrogen deficiency stimulates root growth, allowing the plant to invest in the root system for improvement of nutrient acquisition, and delay of foliage growth until adequate nitrogen is available (Scheible et al, Plant J, 1997, 11(4):671-91). Notably, from about a dozen plants of each type assessed 4-5 days after transfer to N-free MSO, only about 50% of experimental plants started rooting, while 100% of control plants have already rooted. 10 days after transfer to N-free MSO, when all plants have rooted, number of roots per plant was counted. Strikingly, on average control plants had essentially double the number of roots as compared to experimental plants, namely on average 19.9 roots per control plant vs. 9.6 roots on average per experimental plant (FIG. 6B).

These results clearly demonstrate that plants comprising Streptomyces thermoautotrophicus nitrogenase exhibit strong resistance to nitrogen deficiency. While eventually, around three weeks after transfer to N-free MSO medium, the experimental plants also started to succumb and show nitrogen deficiency signs, they clearly showed a robust phenotypical resistance to nitrogen deficiency as compared to control wild type or GUS-only comprising plants. Strategies to further optimize and enhance nitrogenase expression and N₂ fixation in transgenic plants are disclosed in “Promoters, terminators and other genetic elements”, “Enhanced and/or modified nitrogenase activity” and other sections above.

To investigate the mechanism behind nitrogen deficiency resistance of the experimental StNitrogenase plants, their capacity to fix atmospheric nitrogen was tested. Heavy nitrogen isotopes 15N (Sigma, Nitrogen-15N2, 98% atom, cat #364584) have been injected into magenta boxes containing aseptically grown experimental and control plants to a final concentration of 5% (vol/vol). Magenta boxes have been sealed air-tight and incubated for 6-7 days in standard growth conditions under cool-white fluorescent lamps with 16 h light/8 h dark cycle at 26-28° C. Upper plant parts have been collected, dried overnight at 50° C. and ground into a powder using mortar and pestle. To assess 15N enrichment levels, dried plant powder has been encapsulated (5 mg/sample) in tin capsules (COSTECH, cat #NC9464090) and sent for analysis to the Stable Isotope Facility at the University of California, Davis. The results, presented using standardized delta-15N values (Peterson and Fry, Ann Rev Ecol Syst, 1987, 18:293-320), demonstrated sizeable enrichment of approx. ˜20% in 15N content in experimental vs. control plants (average delta 15N of ˜297 vs. ˜367 for control and experimental plants, respectively, FIG. 6C), confirming the ability of Streptomyces thermoautotrophicus nitrogenase comprising plants to fix airborne nitrogen.

Collectively, these results demonstrate that expression of Streptomyces thermoautotrophicus nitrogenase in plants enables them to use airborne N₂ as a source of biologically available nitrogen, leading to strong resistance to nitrogen deficiency as compared to wild type or other control plants.

### Example 4

### Streptomyces thermoautotrophicus Nitrogenase Enables Generation of a Variety of Plant Species Capable of Nitrogen Fixation

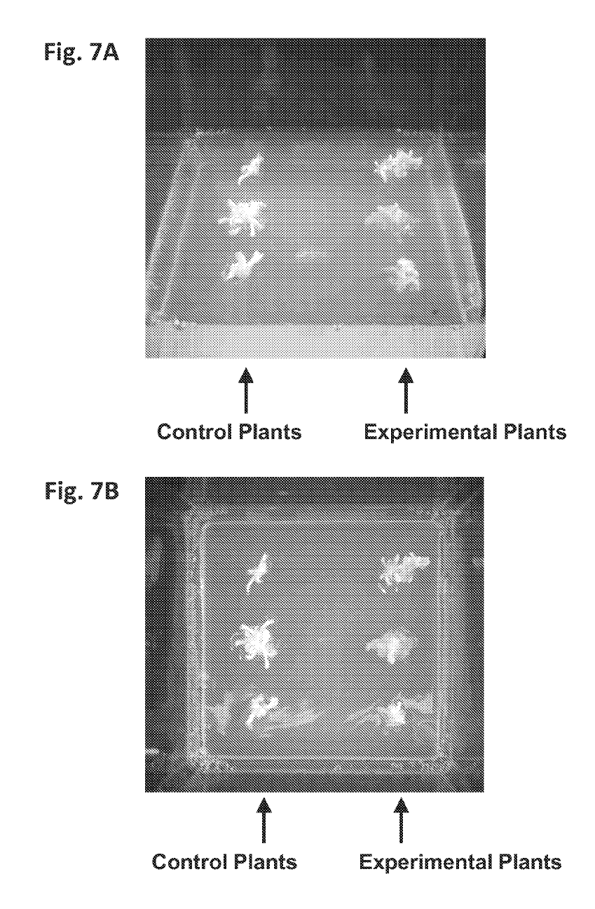

In the preceding examples we focused on generation and characterization of Nicotiana tabacum plants capable of nitrogen fixation. To demonstrate applicability of this technology to other plant species, we transformed additional plant species with Streptomyces thermoautotrophicus nitrogenase. Nicotiana sylvestris (cv. Only the Lonely) transformation has been conducted using constructs and methods described in Examples 1 and 2 above. Plants regenerating from post-bombardment callus have been excised and transferred to N-free MSO medium (as described in Example 3 above) side by side with wild-type N. sylvestris plants regenerated from leaf-derived callus grown on RMOP medium (as described in Example 2, without the antibiotics). As shown in FIG. 7, approximately 7-10 days post transfer, N. sylvestis plants comprising Streptomyces thermoautotrophicus nitrogenase (row of plants on the right side of panels A and B, designated as “Experimental Plants”) retained notably greener appearance, and thus showed considerably reduced effect of nitrogen deprivation, as compared to their wild-type counterparts (row of plants on the left side of panels A and B, designated as “Control Plants”). These results unambiguously demonstrate that Streptomyces thermoautotrophicus nitrogenase enables nitrogen fixation trait in a variety of plant species.

Those skilled in the art will recognize, or be able to ascertain, using no more than routine experimentation, many equivalents to the specific embodiments of the disclosure specifically described herein. Such equivalents are intended to be encompassed within the scope of the following claims.

AMINO ACID AND NUCLEIC ACID SEQUENCES:

SEQ ID NO: 1, Streptomyces thermoautotrophicus St1-L subunit, partial sequence (Ribbe et al, Journal of
Biological Chemistry, 1997, 272 (42):26627-33)
ALPQTELRPMGKPILRKXDP
SEQ ID NO: 2, Streptomyces thermoautotrophicus St1-M subunit, partial sequence (Ribbe et al, Journal of
Biological Chemistry, 1997, 272 (42):26627-33)
MFPNAFKYEAPASVDEAVRLLAEYGYDGKV
SEQ ID NO: 3, Streptomyces thermoautotrophicus St1-S subunit, partial sequence (Ribbe et al, Journal of
Biological Chemistry, 1997, 272 (42):26627-33)
MKIRVKVNGTLYEADVEP
SEQ ID NO: 4, Streptomyces thermoautotrophicus st2-D subunit, partial sequence (Ribbe et al, Journal of
Biological Chemistry, 1997, 272 (42):26627-33)
MFELPPLPYPYDALEPYFDAKKMEIHYYGGHGA
SEQ ID NO: 5, Streptomyces thermoautotrophicus strain St1 putative Mo-hydroxylase (sdnL) gene
(GeneBank KF951061)
GTGGCACTGCCGCAGACTGAACTGCGCCCGATGGGCAAACCGATTCTCCGCAAGGAGGATCCCCGGCTGATCCGC
GGGAAGGGCCGGTTTGTGGACGACATCCTGTTGCCGAATATGCTCCATCTTTGCATCTTGCGGAGCCCGTACGCC
CACGCCCGCATTCGCCGCATCGATACGTCGAAAGCAGAAGCCGCGCCGGGCGTCAAGCTGGTGCTCACGGGAGAA
GATCTGGCCAAGATGAACCTCGCCTGGATGCCGACCTTGGCGGGGGACGTGCAGATGGTGCTGGCGACGGGCAAG
GTCCTGTTCCAGTACCAGGAGGTCGCGGCGGTCGTCGCGGAGACGCGCGCCCAGGCCGAGGACGCGATTCAGCTG
ATCGAGGTCGACTACGAGCCCCTGCCGGTGGTGGTCGATCCGTTCAAGGCGCTGGAGCCGGACGCGCCCATCCTC
CGGGAGGACAAGGAGAAAAAGTCGAACCACATCTGGCACTGGGAGGCGGGCGACCGGGAAGAGACCGACGCGATC
TTCCGCGAAGCGCCGGTCGTCGTCAAGCAGGATGTGCGTTTTCAGCGCGTCCATCCCTCGCCGCTTGAACCGTGC
GGCTGCGTGGCCGACTACAACCCGGCGACGGGGAAGCTCGTGGTCTACGTCACGTCGCAGGCGCCGCACGTCCAC
CGGACGGCGATCGCTTTGACGACGGGCTTCCCCGAACACATGATTCAGGTCATTTCGCCCGATGTGGGCGGCGGG
TTCGGGAACAAGGTGCCCCTCTACCCCGGCTACGTGGTGGCGATCGTCGCTTCCTTGAAGCTGGGAGTCCCCGTG
AAGTGGATCGAGACGCGGACGGAAAACATCGCCAGCACCCACTTCGCCCGCGATTACCACATGACGGCGGAGATC
GCGGCGACGGAAGACGGCAAGATGCTGGCGCTCCGCGTGAAGACGATCGCCGACCACGGCGCGTTCGACGCGACC
GCCAACCCGACCAAATACCCCGCCGGATTGTACAGCATCGTGACGGGGTCGTACGACTTCAAGGCGGCGTTCGTC
GAAGTGGACGGTGTTCATACGAACAAACCGCCGGGCGGTGTGGCCTACCGCTGCTCGTTCCGGGTCACGGAAGCC
TCCTATCTGATTGAACGCGTCGTGGACGTTTTGGCCCGTCGGCTCAAGATGGATCCGGCCGAGTTGCGCCTGCGC
AATTTCATTCGCAAGGAGCAGTTCCCGTACCGCAGCCCGACGGGATGGGTGTACGACAGCGGGGATTACGAAAAG
ACGTTCAAGCTCGCGCTGGAGCGCATCGGATATGAAGAGCTGCGCAAGGAGCAGAAGGAGAAGTGGGCCCGGGGA
GAATTCATGGGCATCGGCATCTCCACCTTCACGGAGATCGTCGGCGCGGGTCCGGCGCACTCCTTCGATATTCTC
GGCATCAAGATGTTCGACAGCGCGGAGATCCGCGTCCATCCGACGGGCAAGGTGATCGCCCGGCTCGGCGTGCGC
CATCAGGGACAGGGGCATGAGACGACGTTCGCCCAGATCATCGCCGAGGAGCTGGGGCTCAGCGTCGACGACGTC
GTGGTCGAAGAAGGCGATACCGACACGGCCCCCTACGGGTTGGGCACGTACGCCAGCCGTTCCACGCCGACGGCC
GGGGCGGCGGCGGCCCTCTGTGCGCGCCGGATCCGGGACAAGGCGCGTAAGATCGCGGCCCATTTGCTGGAGGTC
AACGAAGACGACGTCGTCTGGGACGGCGCCGCCTTTTCGGTCAAGGGACTTCCGGGCCGTTCGGTGACGATGAAA
GATGTGGCCTTTGCCGCCTACACGAACGTGCCCGACGGCATCGAGCCGGGCTTGGAGGCGTCGTACTACTACAAT
CCGCCGAACCTCACCTTCCCCTACGGGGCCTACATCGCCGTGGTCGACATCGACAAGGGAACGGGCGCCGTGAAG
GTGCGGCGGTTCTTGGCCGTCGACGATTGCGGCAACGTGATCAATCCGATGATCGTCGAAGGTCAGGTGCACGGC
GGCCTGACGGAAGGATTTGCGATCGCGTTCATGCAGGACATCCCGTATGACGCCGACGGCAACTGCCTGGCGCCG
AACTGGATGGACTACCTGGTTCCCACCGCTTGGGACACGCCCCAGCTGGAGACGGATCGGACGGTCACGCCCTCG
CCTCACCATCCGCTTGGCGCCAAAGGGGTCGGCGAGTCGCCCAACGTCGGTTCGCCGGCGGCGTTCGTCAATGCG
GTGCTGGACGCGCTGTCGCCGCTCGGCGTAGAACACATCGACATGCCGATCTATCCGTGGAAGGTGTGGAAGATC
TTGCGGGACACGGCATTACGGAGTGATTCGATGGCCATTCCTGCGTCATTCCAGAGCGCGAGGAGGGAAAAGCCC
GGAGGCGGTATAGCCTCCGGGCCCATCAAATGGACAACCTCTGGGAGACAGCGAGGGCGTTGGATGAACGCGCGG
AGCCTTACGTCTGGGTGA
SEQ ID NO: 6, Streptomyces thermoautotrophicus strain St1 putative 2Fe2S-binding dinitrogenase (sdnS)
gene (GeneBank KF951060)
ATGAAGATCCGGGTCAAAGTCAACGGGACGCTGTACGAGGCGGACGTGGAACCGCGGACGCTTCTGGCGTACTTT
CTGCGCGAGGAATTGAAGTTGACGGGCACGCACATCGGCTGCGACACGACCACCTGCGGAGCTTGCACGGTGCTT
TTGGACGGGAAGGCGGTCAAGTCGTGCACGGTCCTCGCGGTGCAGGCGAACGGACGCGAGGTCATGACGGTCGAA
GGGCTGGAAAAAGACGGCCAGCTGCATCCTCTGCAAGTCGCGTTCTGGGAAGAACACGCGCTTCATTGCGGATAT
TGCACGCCCGGTATGTTGATGGCCTCTTACGCGCTGTTGCAAGAAAATCCGATGCCCACCGAGGAAGAGATTCGT
TTTGGATTGTCCGGGAACGTCTGCCGTTGCACCGGTTACATGAACATCGTCAAGGCCGTTCAATCCGCGGCGCGC
AGGCTTTCCGGCGCGTCCGGCGAAGCCGTTGGGGAGGTGGCGACCAGTGGCACTGCCGCAGACTGA
SEQ ID NO: 7, Streptomyces thermoautotrophicus strain St1 putative dinitrogenase (sdnM) gene
(GeneBank KF951059)
GTGTTTCCCAATGCGTTCAAGTACGAGGCGCCGGCATCGGTCGACGAGGCCGTCCGTCTGCTGGCCGAGTACGGC
TACGACGGAAAGGTGTTGGCGGGCGGGCAGAGCTTGCTCCCGATGATGAAGCTGCGCGTCGCGGCGCCGGCCGTG
CTCATCGACATCAACGGCATCGATGCGCTCCAGGGGTGGCGCGAGGTCGACGGGAAACTGCGGGTGGGCGCGATG
ACGCGCCACGCCGAACTGGAGCATGCCAAAGAGCTCCGCGACACGTATCCGCTGTTTTTCCAGACGGCCCGATGG
ATCGCCGATCCGCTCATCCGCAACCGCGGGACCATCGGAGGCTCGCTCGCGCACGCCGATCCCGGCTCCGACTGG
GGGGCGGCGATGATCGCGCTTCGGGCCGAAGTGGAAGCGCGAGGCCCCCAGGGAAGCCGGCTCATTCCCATCGAC
GAATTTTTTGTCGATACGTTTGCAACCGCTTTAAATGAAGACGAACTCGCCGTCGCGGTGCACGTGCCGACGCCG
AAGGGGCCGGCGGCCTCCCGGTATATGAAGCTGGAGCGCCGGGCGGGCGATTTCGCCATCGCCGCGCTCGCCGTC
CACGTCGCCCTCGGAACCGACGGCCGCGTGTCCGAAGCCGGCATCGGCATTTGCGCGTGCGGTCCGATCCCCCTC
CGGGCAGCCAAAGCGGAGGCGGCGCTCATCGGCCGGCCGCTGACGGAAGAGGTCATCGTCGAGGCGTCGAGGCTG
GTTCCGGAAGATGCCGAGCCCGCCGACGATCTGCGAGGAAGCGCGGAATATAAGCGCGACGTGTTGCGCGTGTTT
GCCGCGCGCGCCCTCCGCGACATCGCCAAAGAGCTGCAAGGAAAGGTGGGGATCCAATGA
SEQ ID NO: 8, Streptomyces thermoautotrophicus strain UBT1 superoxide oxidoreductase (sdnO) gene
(GeneBank KF956113)
ATGTTCGAACTGCCGCCGCTTCCGTACCCCTACGACGCGCTGGAGCCGTATTTTGACGCCAAGACGATGGAAATT
CACTACAACGGGCACCACGGCGCTTACGTCAAGAACCTGAACGCCGCCCTCGAAAAATATCCCGCATGGCAAAAT
AAGCCGATTGAAGAGCTGCTTCAGTCCCTCGACCAACTGCCGGAAGACATCCGGACGGCGGTCCGGAACAACGGC
GGGGGCCACTACAACCACAGCTTCTGGTGGCCGATGCTGAAGAAAAACGAAGGGGGCCAGCCGGTCGGCAAGTTT
GCCGAAGCGATCAACCGGGACTTCGGCAGCTTTGAGGCCTTTAAGGACGCCTTTTCCAAGGCGGCGGCGGGACGG
TTCGGAAGCGGCTGGGCGTGGGTCGTCGTCGAACCGGATGGGAAGCTCACCGTCACGACGACGCCGAACCAGGAC
AACCCGGTCATGGAAGGGAAGACGGTCGTCTTCGGCCTCGACGTCTGGGAGCACGCCTACTACCTGAAGTATCAG
AACCGGCGGCCGGAGTACATCCAGGCGTTCTGGAACGTCGTCAACTGGGACGTCGTCAACGAGCGGTACGAAGAA
GCGCTGAAAAAGTTCGGGCGGTAA
SEQ ID NO: 9, Klebsiella pneumoniae DNA for nif gene cluster (Gene Bank X13303)
GGTAACCCGCTACGGCTTGAGATTATCCGCATCCTTGCCGACGGCAGCGAGCAGAGCTGTAACGCCCTGCGTCAC
GAAGATGTGGCGAAGTCGACCATGACCCACCACTGGCGCGTCCTGCGCGACAGCGGTGTGATCTGGCAGCGCCCA
CAGGGGCGGGAGAACTTGATTTCGCTGCGCCGGGAAGATTTAGACGCGCGCTTTCCCGGCCTGCTGGATACGCTG
CTTAAGGTCATGCAGCAGGAGAACTAAAGGCCCGCTACTCCTCGCCGGCCAGCCGCCGATACTGGGCAAAGCGGG
CCCGCGCGTCCTCCTCGGTTCGGCTAAAGAGCGCATCCGCCAGATGCGGCGTCGTTTTGTGCAGCGAGGCGTAGC
GCACTTCGCCAAGCAAAAAGTCGCGGAAGCTCTCCTCCGGCTCTTCGGAATCGAGCATAAACGGCGTCTTACCTT
CCGCTTCCCGCTGCGGATGATAGCGCCACAGGTGCCAGTATCCCGCCTCAACCGCCCGTTTCGCCTCGCGCTGGC
TGCAGCGCATACCGGCTTTCAGCCCGTGGTTAATGCAGGCGGCGTAGGCAATCACCAGCGACGGTCCCGGCCAGG
CTTCGGCCTCGGCGATCGCCCGTAGGGTCTGATCTTTATCAGCGCCCATCGCGACCTGGGCCACGTACACATTGC
CGTAGCTCATCGCCATCATGCCGAGATCTTTTTTCCGCGTGCGTTTGCCCTGCGCGGCAAACTTCGCGATGGCCG
CCACCGGGGTCGATTTAGACGACTGGCCGCCGGTATTGGAGTAAACCTCGGTGTCAAACACCAGAATATTGACGT
CTTCCCCGCTCGCCAGCACGTGATCGAGACCGCCGAAGCCGATATCGTAGGCCCAGCCGTCGCCGCCGAAAATCC
ACTGCGAACGACGAACAAAATAGTCGCGGTTCTGCCACAGCTGCTCCAACAGCGGCACGCCCTCTTTTTCCGCCG
CCAGCCGTTCGCTGAGCCGGTCCGCGCGCTCGCGGGTGCCCTCGCCTTCATCCTGCTTCGCCAGCCACTGGCGCA
TTGCGTCGCTAAGTTCGTCGCTGACCGGTAGCGCCAGCGCGGCGGTCATATCATCGGCGATTTGTTGACGCACCG
CCTGGCCGCCGAGCATCATGCCGAGGCCAAACTCCGCATTATCCTCAAACAGCGAGTTCGCCCATGCCGGGCCAT
GGCCGCGGTGGTTGGTGGTATAGGGAATCGACGGCGCGCTGGCTCCCCAGATAGAAGAGCAGCCGGTGGCGTTAG
CGATCAGCATCCGGTCGCCAAACAGCTGGGTTATCAGGCGGGCATAAGGCGTTTCACCGCATCCCGCGCAGGCGC
CGGAAAACTCCAGCAGCGGGGTTTCAAACTGGCTGCCTTTGACCGTCGTCTTACGAAACGGATTGCTCTTCGGCG
TCAGCGCCAGCGCATAGTCCCAGACCGGCGCCATCTGACGCTGGCTATCGAGAGACTGCATTTTTAACGCCTTGC
CGCGCGCGGGACAGATATCCACGCAGTTGCCGCAGCCGGAACAATCCAGCGGCGAGATAGCCAGATGGTAGTGAT
ACTCCTTCGCTCCCTGCGCGGGTTTGCTCAGCAGCCCAACCGGCGCGGCGTCATGCTCTTCGCCGTTGAGCAGCG
CCGGGCGGATCGCCGCATGCGGGCAGATAAAGGCGCACTGGTTACACTGCGTGCAGCCCTCCGGCTGCCAGACCG
GCACTTCCAGCGCGATCCCGCGTTTCTCCCACGCGGCGGTGCCCGAAGGAAAGGTCCCGTCCTCCATACCGACGA
ACGCGCTCACCGGCAGCTGGTCGCCGCACTGGCGGTTCATCGGCTGCAGAATATCGCGGATGAAATCCGGCATCA
TGGCTGATGCTTGCGCCGCGGGTTCATCCAGCGTCGCCCAGTGCGCCGGAATCGTCACCTGATGCAGCGAGGCCA
TGCCCAGCTCGATCGCCCGCTGGTTCATCTCAATCACCGCCGCCCCTTTGCTGCCGTAGCTTTTTTCAACCGCCT
GCTTGAGGTAATCCGCCGCGGTCTGCGGGTCGATAATCGCCGCCAGCTTAAAGAACGCCGCCTGCATCAGCATAT
TAAAGCGCCCGCCCAGCCCGAGCTCGCGGGCGATATCCACGGCGTTCAGGGTATAAAAATGGATATTTTCCCGCG
CCAGATAGCGTTTAAAGCCGACCGGCAGATGCTGCTCCAGCTCCGCATCGGACCAGCTGCAGTTGAGTAAAAAGG
TCCCGCCCGGCTTTAATCCGTCCAGCAGATCGTAGCGCTCAACGTAGGACTGCTGCGAACAGGAGATAAAATCGG
CCCGATGGATCAGGTAGGGCGAATTGATCGGCCGGTCGCCGAAGCGTAAATGTGAAACGGTAATGCCGCCGGATT
TTTTCGAGTCATAAGAAAAGTAGGCCTGCGCGTAGAGCGGCGTTTTATCGCCGATAATTTTGATCGCGCTTTTAT
TGGCCCCGACGGTGCCGTCCGAGCCCATGCCCCAAAATTTACAGGCGGTGATGCCGTCATGCGAGACCGCCAGCG
TCTGCTGGGCCGGCGGTAACGAAGTAAAGGTTACATCATCGACAATCCCGAGGGTAAACCCGTCCATCGGCAGCG
GTTTATTGAGGTTATCAAAGACGGCCGCGATATCGTTGGGCAGAACATCCTTCCCGCCAAGCGCATAGCGGCCGC
CGACGATTAGCGGCGCATCGTCGTGGTGGTAGAAGGCGTTTTTCACATCCAGGCACAGCGGTTCAGCCTGAGCGC
CGGGCTCTTTGGTACGGTCAAGGACGGCAATCCGCTGCACGGTTTTCGGCAGCTGGGCGAAGAAGTGGGCCAGCG
AAAAAGGGCGAAACAGATGCACGCTGAGCAGCCCGACCTTCTCTCCCGCCGCGTTCAGCGTATCCACCACTTCCT
GAACGGTATCGCAGACCGATCCCATTGCGATAATCACCCGTTCGGCATCCGCCGCGCCGGTATAGTTAAACAGAT
GATACTCCCGGCCGGTGAGCGCGCTGATTTGCGTCATATAGCTTTCGACAATGTCGGGCAGCGCCTGATAAAAAC
GGTTGCCCGCCTCCCGCTCCTGGAAGTAGATATCCGGGTTCTGCGCCGTTCCGCGGATGACCGGATGATCCGGAT
GCAGCGCGTTACGGCGGAAGCTGTCGAGCGCGGGCCGGTCCAGCAGCGTCGCCAGCTGCTCATATTCCAACACCT
CGATTTTTTGAATTTCGTGCGAGGTGCGAAAACCGTCGAAGAAGTTAACAAACGGGATGCGTCCCTTAATCGCCG
CCAGATGCGCCACCGCCGACAAATCCATCACCTGCTGCACGTTGTTCTCCGCCAGCATCGCGCAGCCGGTCTGGC
GGACCGCCATCACATCCTGGTGATCGCCAAAAATATTCAGCGAATTGGTCGCCAGCGCCCGGGCGCTGACGTGAA
AGACGCCCGGCAGCAGTTCACCGGCGATTTTGTACATGTTGGGGATCATCAGCAGCAGCCCCTGGGAGGCCGTAT
AGGTGGTGGTGAGCGCCCCGGCCTGCAGCGCGCCGTGGACCGCGCCTGCCGCGCCGGCCTCCGACTGCATCTCCA
TTAAGCGCACCGGCTGGCCAAAAAGGTTCTTTTTCCCCTGCGCCGCCCACTCGTCGACGTTTTCCGCCATCGGCG
TGGAGGGGGTTATGGGGTAAATCGCCGCGACCTCGGTAAAGGCATAAGAGATCCAGGCCGCCGCGGCGTTGCCAT
CCATTGTTTTCATTTTTCCGGACATTGTTCAATCCTCGAAGGTGAGAGGCATCTTCGCCGCCTCAAATAAGCGGC
AAACCCAGTTGTTGCCTCAAGCACAGCCTGTGCCAGCTCGCGGATGACAGAAGAGTTAGCGCGAATTCAACGCGT
TATGAAGAGAGTCGCCGCGCAGCGCGCCAAGAGATTGCGTGGAATAAGACACAGGGGGCGACAAGCTGTTGAACA
GGCGACAAAGCGCCCATGGCCCCGGCAGGCGCAATTGTTCTGTTTCCCACATTTGGTCGCCTTATTGTGCCGTTT
TGTTTTACGTCCTGCGCGGCGACAAATAACTAACTTCATAAAAATCATAAGAATACATAAACAGGCACGGCTGGT
ATGTTCCCTGCACTTCTCTGCTGGCAAACACTCAACAACAGGAGAAGTCACCATGACCATGCGTCAATGCGCTAT
TTACGGTAAAGGCGGTATCGGTAAATCCACCACCACGCAGAACCTCGTCGCCGCGCTGGCGGAGATGGGTAAGAA
AGTGATGATCGTCGGCTGCGATCCGAAGGCGGACTCCACCCGTCTGATTCTGCACGCCAAAGCACAGAACACCAT
TATGGAGATGGCCGCGGAAGTCGGCTCGGTCGAGGACCTCGAACTCGAAGACGTGCTGCAAATTGGCTACGGCGA
TGTGCGCTGCGCGGAATCCGGCGGCCCGGAGCCAGGCGTCGGCTGCGCGGGACGCGGCGTGATCACGGCGATCAA
CTTTCTTGAAGAAGAAGGCGCCTACGAGGACGATCTCGATTTCGTGTTCTATGACGTGCTCGGCGACGTGGTCTG
CGGCGGCTTCGCCATGCCGATCCGCGAAAACAAAGCCCAGGAGATCTACATCGTCTGCTCCGGCGAAATGATGGC
GATGTACGCGGCCAACAATATCTCCAAAGGGATCGTTAAATACGCCAAATCCGGCAAGGTGCGCCTCGGCGGCCT
GATCTGTAACTCACGTCAGACCGACCGTGAAGACGAACTGATTATTGCCCTGGCGGAAAAGCTCGGTACCCAGAT
GATCCACTTTGTGCCCCGCGACAACATCGTGCAGCGCGCGGAGATCCGCCGCATGACGGTTATCGAGTACGACCC
CGCCTGTAAACAGGCCAACGAATACCGCACCCTGGCGCAGAAGATCGTCAACAACACCATGAAAGTGGTGCCGAC
GCCCTGCACCATGGATGAGCTGGAATCGCTGCTGATGGAGTTCGGCATCATGGAAGAGGAAGACACCAGCATCAT
TGGCAAAACCGCCGCCGAAGAAAACGCGGCCTGAGCACAGGACAATTATGATGACCAACGCAACGGGCGAACGTA
ATCTGGCGCTGATCCAGGAAGTCCTGGAGGTGTTCCCGGAAACCGCGCGAAAAGAGCGCAGAAAGCACATGATGG
TCAGCGATCCGAAAATGAAGAGCGTCGGCAAGTGCATTATCTCTAACCGCAAATCACAACCCGGCGTAATGACCG
TACGCGGCTGCGCCTACGCCGGTTCCAAAGGGGTGGTATTTGGGCCGATTAAGGATATGGCCCATATTTCGCACG
GACCGGCTGGCTGCGGCCAGTATTCCCGCGCCGAACGACGCAACTACTACACCGGAGTCAGCGGCGTCGATAGCT
TCGGCACGCTGAACTTCACCTCTGATTTTCAGGAGCGCGACATCGTCTTCGGCGGCGATAAAAAGCTCAGCAAGC
TGATTGAAGAGATGGAGTTGCTGTTCCCGCTCACCAAAGGGATCACCATTCAGTCGGAATGCCCGGTGGGGCTGA
TCGGTGATGATATCAGCGCGGTGGCCAACGCCAGCAGCAAGGCGCTGGATAAACCGGTGATCCCGGTACGCTGCG
AAGGCTTTCGCGGCGTGTCGCAGTCTCTGGGGCACCATATCGCCAACGACGTGGTGCGCGACTGGATCCTGAACA
ATCGCGAAGGACAGCCGTTTGAAACCACCCCTTACGATGTGGCGATCATCGGCGACTACAACATCGGCGGCGACG
CCTGGGCCTCGCGCATTCTGCTGGAAGAGATGGGGCTACGGGTAGTCGCGCAGTGGTCCGGCGACGGCACGCTGG
TGGAGATGGAGAATACCCCATTCGTCAAGCTGAACCTGGTTCACTGCTACCGTTCGATGAACTATATCGCCCGCC
ATATGGAGGAGAAACATCAGATTCCGTGGATGGAGTACAACTTCTTCGGGCCGACCAAAATCGCCGAATCGCTGC
GCAAAATCGCCGACCAGTTCGACGATACCATTCGCGCGAACGCCGAAGCGGTGATCGCCCGGTATGAGGGGCAGA
TGGCGGCGATTATCGCCAAATATCGCCCGCGCCTGGAGGGGCGTAAGGTGCTGCTCTATATCGGAGGCCTGCGGC
CGCGCCACGTTATTGGCGCCTATGAGGATCTCGGGATGGAGATCATCGCCGCCGGCTACGAGTTTGCCCATAACG
ATGATTACGACCGCACCCTGCCGGATCTGAAAGAGGGCACGCTGCTGTTCGATGACGCCAGCAGCTACGAGCTGG
AAGCGTTCGTCAAGGCGCTGAAGCCCGACCTTATCGGCTCCGGCATCAAGGAAAAATATATCTTCCAGAAAATGG
GCGTGCCGTTCCGCCAGATGCACTCGTGGGACTATTCCGGCCCGTACCACGGCTACGATGGTTTCGCCATTTTCG
CCCGCGATATGGATATGACCCTGAACAACCCGGCGTGGAACGAACTGACCGCTCCGTGGCTGAAGTCTGCGTGAT
TGCCCACTCACTGTCCCGTCTGTTCACCGATTTGTGGCGCGGGAGGAGAACACCATGAGCCAAACGATTGATAAA
ATTAATAGCTGTTATCCGCTATTCGAACAGGATGAATACCAGGAGCTGTTCCGCAATAAGCGGCAGCTGGAAGAG
GCGCACGATGCGCAGCGCGTGCAGGAGGTCTTTGCCTGGACCACCACCGCCGAGTATGAAGCGCTGAATTTCCGA
CGCGAGGCGCTGACCGTTGACCCGGCGAAAGCCTGCCAGCCGCTTGGCGCGGTGCTTTGCTCGCTGGGATTTGCC
AACACCCTGCCGTATGTGCACGGCTCTCAGGGGTGCGTGGCCTACTTTCGCACCTATTTTAACCGCCATTTCAAA
GAGCCGATCGCCTGCGTCTCCGACTCGATGACCGAAGACGCGGCGGTCTTCGGCGGCAACAACAATATGAACCTG
GGCCTGCAGAACGCCAGCGCGCTGTACAAACCGGAGATCATTGCGGTGTCCACCACCTGCATGGCGGAAGTTATC
GGCGATGACCTGCAGGCGTTTATCGCCAACGCTAAAAAAGATGGCTTCGTCGACAGCAGCATCGCCGTGCCCCAC
GCCCATACGCCAAGCTTTATCGGCAGCCACGTCACCGGCTGGGATAACATGTTTGAAGGCTTCGCCAAAACCTTC
ACTGCGGACTACCAGGGGCAGCCGGGCAAATTGCCGAAGCTCAATCTGGTGACCGGCTTTGAAACCTATCTCGGC
AACTTCCGCGTATTAAAGCGGATGATGGAACAGATGGCGGTGCCGTGCAGCCTGCTCTCCGATCCGTCGGAAGTT
CTCGACACGCCCGCCGACGGTCACTATCGGATGTATTCCGGCGGCACCACGCAGCAGGAGATGAAAGAGGCCCCT
GACGCCATCGATACGCTGCTCCTGCAGCCGTGGCAGCTGCTGAAGAGCAAAAAAGTGGTGCAGGAGATGTGGAAC
CAGCCCGCCACCGAGGTCGCCATTCCGCTGGGGCTGGCCGCCACCGATGAACTGCTGATGACCGTCAGCCAGCTT
AGCGGCAAGCCGATTGCCGACGCCCTCACCCTTGAGCGCGGCCGGCTGGTTGACATGATGCTCGACTCCCACACC
TGGCTGCACGGCAAGAAGTTTGGCCTGTACGGCGATCCGGACTTCGTGATGGGCCTCACCCGCTTCCTGCTGGAG
CTGGGCTGCGAGCCAACGGTGATCCTGAGCCATAACGCCAACAAACGCTGGCAAAAAGCGATGAACAAAATGCTC
GATGCCTCGCCGTACGGGCGCGATAGCGAAGTGTTTATCAACTGCGATTTGTGGCACTTCCGTTCGCTGATGTTC
ACCCGTCAGCCGGACTTTATGATCGGCAACTCCTACGGCAAGTTTATCCAGCGCGATACCCTGGCGAAGGGTAAA
GCCTTTGAAGTGCCGCTTATCCGCCTCGGCTTTCCGCTGTTCGACCGCCACCATCTGCACCGCCAGACAACCTGG
GGTTATGAAGGGGCGATGAACATTGTGACGACGCTGGTGAACGCCGTGCTGGAGAAACTGGATAGCGATACCAGC
CAGCTGGGCAAAACCGATTACAGCTTCGATCTCGTCCGTTAACCATCAGGTGCCCCGCGTCATGCGGCGGCAGGA
GGGAGTATGCCCATCGTGATTTTCCGTGAGCGCGGCGCGGACCTGTACGCCTATATCGCGAAACAGGATCTGGAA
GCGCGAGTGATCCAGATTGAGCATAACGACGCTGAACGCTGGGGCGGCGCGATTTCGCTGGAGGGGGGACGCCGC
TACTACGTGCATCCGCAGCCGGGGCGTCCCGTCTTTCCGATAAGCCTGCGCGCGACGCGCAATACCTTGATATAA
GGAGCTAGTGATGTCCGACAACGATACCCTATTCTGGCGTATGCTGGCGCTGTTTCAGTCTCTGCCGGACCTACA
GCCGGCGCAAATCGTCGACTGGCTGGCGCAGGAGAGCGGCGAGACGCTGACGCCAGAGCGTCTGGCGACCCTGAC
CCAGCCGCAGCTGGCCGCCAGCTTTCCCTCCGCGACGGCGGTGATGTCCCCCGCTCGCTGGTCGCGGGTGATGGC
GAGCCTGCAGGGCGCGCTGCCCGCCCATTTACGCATCGTTCGCCCTGCCCAGCGCACGCCGCAGCTGCTGGCGGC
ATTTTGCTCCCAGGATGGGCTGGTGATTAACGGCCATTTCGGCCAGGGACGACTGTTTTTTATCTACGCGTTCGA
TGAACAAGGCGGCTGGTTGTACGATCTGCGCCGCTATCCCTCCGCCCCCCACCAGCAGGAGGCCAACGAAGTGCG
CGCCCGGCTTATTGAGGACTGTCAGCTGCTGTTTTGCCAGGAGATAGGCGGGCCCGCCGCCGCGCGGCCGATCCG
CCATCGCATCCACCCGATGAAAGCGCAGCCCGGGACGACGATTCAGGCACAGTGCGAGGCGATCAATACGCTGCT
GGCCGGCCGTTTGCCGCCGTGGCTGGCGAAGCGGCTTAACAGGGATAACCCTCTGGAAGAACGCGTTTTTTAATC
CCTGTTTTGTGCTTGTTGCCCGCTGACCCCGCGGGCTTTTTTTCGCGTATGGACGCTCTTCCCCACGTTACGCTC
AGGGGAATATTCCGTTCACGGTTGTTCCGGGCTTCTTGATGCGCCTAACCCCCTCGCTGCCAGCCTTTCATCAAC
AAATAGCCATCCCAGCGCGATAGGTCATAAAGCATCACATGCCGCCATCCCTTGTCCGATTGTTGGCTTTGTCGC
AAAGCCAACAACCTCTTTTCTTTAAAAATCAAGGCTCCGCTCTGGAGCGCGAATTGCATCTTCCCCCTCATCCCC
CACCGTCAACGAGGTCACTATGAAGGGAAATGAAATTCTGGCGCTGCTGGATGAACCGGCCTGTGAACACAACCA
TAAACAAAAATCCGGCTGCAGCGCGCCCAAACCCGGCGCCACCGCCGCGGGCTGCGCGTTCGACGGCGCGCAGAT
AACCCTGCTGCCCATCGCCGACGTGGCGCATCTGGTCCACGGCCCCATCGGCTGCGCCGGAAGCTCATGGGATAA
CCGCGGCAGCGCCAGCTCCGGCCCCACCCTTAATCGGCTCGGGTTCACCACCGATCTCAACGAACAGGACGTGAT
TATGGGCCGCGGCGAACGCCGACTGTTTCACGCCGTGCGCCATATCGTCACCCGCTATCATCCGGCGGCGGTCTT
TATCTACAACACCTGCGTACCGGCCATGGAGGGCGATGACCTGGAAGCGGTATGCCAGGCCGCGCAGACCGCCAC
CGGCGTACCGGTTATCGCTATTGACGCCGCCGGTTTCTACGGCAGTAAAAATCTCGGTAACCGGCCGGCGGGCGA
CGTCATGGTCAAACGGGTCATCGGCCAGCGCGAGCCCGCCCCCTGGCCGGAGAGCACGCTCTTTGCCCCGGAGCA
GCGTCACGATATTGGCCTGATTGGCGAATTCAATATTGCCGGCGAGTTCTGGCATATTCAGCCGCTGCTCGACGA
ACTGGGGATCCGCGTGCTCGGCAGCCTCTCCGGTGATGGCCGCTTCGCCGAGATCCAGACCATGCACCGGGCGCA
GGCCAATATGCTGGTCTGCTCGCGGGCGTTAATTAACGTCGCCAGAGCCCTGGAGCAGCGCTACGGCACGCCGTG
GTTCGAAGGCAGCTTTTACGGGATCCGCGCCACCTCTGACGCCCTGCGCCAGCTGGCGGCGCTGCTGGGCGACGA
CGACCTTCGCCAGCGCACCGAAGCGCTGATTGCGCGGGAGGAACAGGCGGCGGAACTGGCGCTACAGCCGTGGCG
CGAACAGCTGCGCGGCCGCAAAGCGCTGCTCTATACCGGCGGGGTGAAATCCTGGTCGGTGGTATCGGCGCTGCA
GGATTTGGGCATGACCGTGGTGGCAACCGGCACGCGTAAATCCACCGAAGAGGATAAACAGCGGATCCGCGAGCT
GATGGGCGAAGAGGCGGTAATGCTGGAAGAGGGCAACGCCCGCACGCTGCTGGATGTGGTCTATCGCTATCAGGC
CGACCTGATGATTGCCGGCGGACGCAATATGTACACCGCCTATAAAGCCAGGCTGCCGTTTCTCGATATCAATCA
GGAGCGCGAACACGCCTTCGCTGGCTATCAGGGGATCGTCACCCTCGCCCGCCAGCTGTGTCAGACCATCAACAG
CCCCATCTGGCCGCAAACCCATTCTCGCGCCCCGTGGCGCTAAGGAGCTCACCATGGCAGACATTTTCCGCACCG
ATAAGCCGCTGGCGGTCAGCCCCATCAAAACCGGCCAGCCGCTCGGCGCAATCCTCGCCAGCCTCGGGATCGAAC
ACAGCATCCCTCTGGTCCACGGCGCGCAGGGGTGCAGCGCCTTCGCCAAAGTCTTTTTTATTCAACATTTCCACG
ACCCGGTTCCCCTGCAGTCGACGGCGATGGACCCCACGTCGACGATTATGGGCGCGGACGGCAATATTTTTACCG
CCCTGGATACCCTCTGCCAGCGCAACAATCCGCAGGCTATCGTACTGCTCAGCACCGGGCTGTCGGAGGCCCAGG
GCAGCGATATTTCCCGCGTGGTTCGCCAGTTTCGCGAAGAGTATCCCCGGCATAAGGGGGTGGCGATATTGACGG
TTAACACGCCGGATTTTTATGGCTCCATGGAGAACGGCTTCAGCGCGGTGTTAGAGAGCGTCATTGAGCAGTGGG
TGCCGCCGGCGCCGCGCCCGGCTCAGCGCAATCGCCGGGTCAATCTGCTGGTCAGCCATCTCTGTTCGCCGGGCG
ATATCGAGTGGCTGCGCCGATGCGTCGAAGCCTTTGGTCTGCAGCCGATAATCCTGCCGGACCTGGCGCAATCGA
TGGACGGCCACCTGGCGCAGGGCGATTTCTCGCCGCTGACCCAGGGCGGGACGCCGCTGCGCCAGATAGAGCAGA
TGGGGCAAAGCCTGTGCAGCTTCGCCATTGGCGTCTCCCTTCATCGCGCCTCATCGCTGCTGGCCCCGCGCTGCC
GCGGCGAGGTTATCGCCCTGCCGCACCTGATGACCCTCGAACGCTGCGACGCCTTTATTCATCAACTGGCGAAAA
TTTCCGGACGCGCCGTTCCCGAGTGGCTGGAACGCCAGCGCGGCCAGCTACAGGATGCGATGATCGACTGCCATA
TGTGGCTCCAGGGCCAGCGCATGGCGATAGCGGCGGAAGGCGATTTGCTGGCGGCGTGGTGTGATTTCGCCAACA
GCCAGGGGATGCAGCCCGGCCCGCTGGTGGCCCCTACCGGTCATCCCAGCCTGCGCCAGCTGCCGGTGGAACGGG
TGGTGCCGGGGGATCTGGAGGATCTGCAAACCCTGCTGTGCGCGCATCCCGCCGACCTGCTGGTGGCGAACTCGC
ACGCCCGCGACCTGGCGGAGCAGTTTGCGCTGCCGCTGGTGCGCGCGGGTTTTCCGCTCTTTGACAAGCTCGGCG
AATTCCGCCGGGTGCGACAGGGGTATAGCGGGATGCGCGATACGCTGTTTGAGCTGGCAAACCTGATACGCGAGC
GTCACCACCACCTCGCCCACTACCGATCGCCGCTGCGCCAGAACCCCGAATCGTCACTCTCCACAGGAGGCGCTT
ATGCCGCCGATTAACCGTCAGTTTGATATGGTCCACTCCGATGAGTGGTCTATGAAGGTCGCCTTCGCCAGCTCC
GACTATCGTCACGTCGATCAGCACTTCGGCGCTACCCCGCGGCTGGTGGTGTACGGCGTCAAGGCGGATCGGGTC
ACTCTCATCCGGGTGGTTGATTTCTCGGTCGAGAACGGCCACCAGACGGAGAAGATCGCCAGGCGGATCCACGCC
CTGGAGGATTGCGTCACGCTGTTCTGCGTGGCGATTGGCGACGCGGTTTTTCGCCAGCTGTTGCAGGTGGGCGTG
CGTGCCGAACGCGTTCCCGCCGACACCACCATCGTCGGCTTACTGCAGGAGATTCAGCTCTACTGGTACGACAAA
GGGCAGCGCAAAAATCAGCGCCAGCGCGACCCGGAGCGCTTTACCCGTCTGCTGCAGGAGCAGGAGTGGCATGGG
GATCCGGACCCGCGCCGCTAGCCGTGTCGTTTCTGTGACAAAGCCCACAAAACATCGCGACACTGTAGGACGAAC
CTTGTCAGGACTAATACACAACCATTTGAAAAATATTAATTTTATTCTCTGGTATCGCAATTGCTAGTTCGTTAT
CGCCACCGCGCTTCCGCGGTGAACCGCGCCCCGGCGTTTTCCGTCAACATCCCTGGAGCTGACAGCATGTGGAAT
TACTCCGAGAAAGTGAAAGACCATTTTTTTAACCCCCGCAATGCGCGCGTGGTGGACAACGCCAACGCGGTAGGC
GACGTCGGTTCGTTAAGCTGCGGCGACGCCCTGCGCCTGATGCTGCGCGTCGACCCGCAAAGCGAAATCATTGAG
GAGGCGGGCTTCCAGACCTTCGGCTGCGGCAGCGCCATCGCCTCCTCCTCCGCGCTGACGGAGCTGATTATCGGC
CATACCCTCGCCGAAGCCGGGCAGATAACCAATCAGCAGATTGCCGATTATCTCGACGGACTGCCGCCGGAGAAA
ATGCACTGCTCGGTGATGGGCCAGGAGGCCCTGCGCGCGGCCATCGCCAACTTTCGCGGCGAAAGCCTTGAAGAG
GAGCACGACGAGGGCAAGCTGATCTGCAAATGCTTCGGCGTCGATGAAGGGCATATTCGCCGCGCGGTACAGAAC
AACGGGCTGACCACCCTTGCCGAGGTGATCAACTACACCAAAGCGGGCGGCGGCTGCACCTCTTGCCACGAAAAA
ATCGAGCTGGCCCTGGCGGAGATCCTCGCCCAGCAGCCGCAGACGACGCCAGCCGTGGCCAGCGGCAAAGATCCG
CACTGGCAGAGCGTCGTCGATACCATCGCAGAACTGCGGCCGCATATTCAGGCCGACGGCGGCGATATGGCGCTA
CTCAGCGTCACCAACCACCAGGTGACCGTCAGCCTCTCCGGCAGCTGTAGCGGCTGCATGATGACCGATATGACC
CTGGCCTGGCTGCAGCAAAAACTGATGGAACGTACCGGCTGTTATATGGAAGTGGTGGCGGCCTGAGCCGGCGTT
AACTGACCCAAGGGGGACAAGATGAAACAGGTTTATCTCGATAACAACGCCACCACCCGTCTGGACCCGATGGTC
CTGGAAGCGATGATGCCCTTTTTGACCGATTTTTACGGCAACCCCTCGTCGATACACGATTTTGGCATTCCGGCC
CAGGCGGCTCTGGAACGCGCGCATCAGCAGGCTGCGGCGCTGCTGGGCGCGGAGTATCCCAGCGAGATCATCTTT
ACCTCCTGCGCCACCGAAGCCACCGCCACCGCCATCGCCTCGGCGATCGCCCTGCTGCCTGAGCGTCGCGAAATC
ATCACCAGCGTGGTCGAACATCCGGCGACGCTGGCGGCCTGCGAGCACATGGAGCGCGAGGGCTACCGGATTCAT
CGCATCGCGGTAGATGGCGAGGGGGCGCTGGACATGGCGCAGTTCCGCGCGGCGCTCAGCCCGCGCGTCGCGTTG
GTCAGCGTGATGTGGGCGAATAACGAAACCGGGGTGCTTTTCCCGATCGGCGAAATGGCGGAGCTGGCCCATGAA
CAAGGGGCGCTGTTTCACTGCGATGCGGTGCAGGTGGTCGGGAAAATACCGATCGCCGTGGGCCAGACCCGCATC
GATATGCTCTCCTGCTCGGCGCATAAGTTCCACGGGCCAAAAGGCGTAGGCTGTCTTTATCTGCGGCGGGGAACG
CGCTTTCGCCCGCTGCTGCGCGGCGGTCACCAGGAGTACGGTCGGCGAGCCGGGACAGAAAATATCTGCGGAATC
GTCGGCATGGGCGCGGCCTGCGAGCTGGCGAATATTCATCTGCCGGGAATGACGCATATCGGCCAATTGCGCAAC
AGGCTGGAGCATCGCCTGCTGGCCAGCGTGCCGTCGGTCATGGTGATGGGCGGCGGCCAGCCGGCGGTGCCCGGC
ACGGTGAATCTGGCCTTTGAGTTTATTGAAGGTGAAGCCATTCTGCTGCTGTTAAACCAGGCCGGGATCGCCGCC
TCCAGCGGCAGCGCCTGCACCTCAGGCTCGCTGGAACCCTCCCACGTGATGCGGGCGATGAATATCCCCTACACC
GCCGCCCACGGCACCATCCGCTTTTCTCTCTCGCGCTACACCCGGGAGAAAGAGATCGATTACGTCGTCGCCACG
CTGCCGCCGATTATCGACCGGCTGCGCGCGCTGTCGCCCTACTGGCAGAACGGCAAGCCGCGCCCGGCGGACGCC
GTATTCACGCCGGTTTACGGCTAAGGCGGAGGTGGCTGATGGAACGCGTGCTGATTAACGATACCACCCTGCGCG
ACGGCGAGCAGAGCCCCGGCGTCGCCTTTCGCACCAGCGAAAAGGTCGCCATTGCCGAGGCGCTTTACGCCGCAG
GAATAACGGCGATGGAGGTCGGCACCCCGGCGATGGGCGACGAGGAGATCGCGCGGATCCAGCTGGTGCGTCGCC
AGCTGCCCGACGCGACCCTGATGACCTGGTGTCGGATGAACGCGCTGGAGATCCGCCAGAGCGCCGATCTGGGCA
TCGACTGGGTGGATATCTCGATTCCGGCTTCGGATAAGCTGCGGCAGTACAAACTGCGCGAGCCGCTGGCGGTGC
TGCTGGAGCGGCTGGCGATGTTTATCCATCTTGCGCATACCCTCGGCCTGAAGGTATGCATCGGCTGCGAGGACG
CCTCGCGGGCCAGCGGCCAGACCCTGCGCGCTATCGCCGAGGTCGCGCAGCAATGCGCCGCCGCCCGCCTGCGCT
ATGCCGATACGGTCGGCCTGCTCGACCCTTTTACCACCGCGGCGCAAATCTCGGCCCTGCGCGACGTCTGGTCCG
GCGAAATCGAAATGCATGCCCATAACGATCTGGGTATGGCGACCGCCAATACGCTGGCGGCGGTAAGCGCCGGGG
CCACCAGCGTGAATACGACGGTCCTCGGTCTCGGCGAGCGGGCGGGCAACGCGGCGCTGGAAACCGTCGCGCTGG
GCCTTGAACGCTGCCTGGGCGTGGAGACCGGCGTGCATTTTTCGGCGCTGCCCGCGTCCTGTCAGAGGGTCGCGG
AAGCCGCGCAGCGCGCCATCGACCCGCAGCAGCCGCTGGTCGGCGAGCTGGTGTTTACCCATGAGTCAGGTGTCC
ACGTGGCGGCGCTGCTGCGGCACAGCGAGAGCTACCAGTCCATCGCCCCTTCCCTGATGGGCCGCAGCTACCGGC
TGGTGCTGGGCAAACACTCCGGGCGTCAGGCGGTCAACGGCGTTTTTGACCAGATGGGCTATCACCTCAACGCCG
CGCAGATTAACCAGCTGCTGCCCGCCATCCGCCGCTTCGCCGAGAACTGGAAGCGCAGCCCGAAAGATTACGAGC
TGGTGGCTATCTACGACGAGCTGTGCGGTGAATCCGCTCTGCGGGCGAGGGGGTAATGATGGAGTGGTTTTATCA
AATTCCCGGCGTGGACGAACTTCGCTCCGCCGAATCTTTTTTTCAGTTTTTCGCCGTCCCCTATCAGCCCGAGCT
GCTTGGCCGCTGCAGCCTGCCGGTGCTGGCAACGTTTCATCGCAAACTCCGCGCGGAGGTGCCGCTGCAAAACCG
GCTCGAGGATAACGACCGCGCGCCCTGGCTGCTGGCGCGAAGACTGCTCGCGGAGAGCTATCAGCAACAGTTTCA
GGAGAGCGGAACATGAGACCGAAATTCACCTTTAGCGAAGAGGTCCGCGTCGTACGCGCGATTCGTAACGACGGC
ACCGTGGCGGGCTTCGCGCCCGGCGCGCTGCTGGTCAGGCGCGGCAGCACCGGCTTTGTGCGCGACTGGGGCGTT
TTTTTGCAAGATCAGATTATCTACCAGATCCACTTTCCGGAAACCGATCGGATCATCGGCTGCCGCGAGCAGGAG
CTGATCCCCATCACCCAGCCGTGGCTGGCCGGAAATTTGCAATACAGGGATAGCGTGACCTGCCAGATGGCGCTC
GCGGTCAACGGCGATGTGGTCGTGAGCGCCGGCCAGCGGGGACGCGTTGAGGCTACCGATCGGGGAGAGCTCGGC
GACAGCTACACCGTCGACTTTAGCGGCCGCTGGTTCAGGGTCCCGGTGCAGGCCATCGCCCTTATAGAGGAAAGA
GAAGAATGAACCCGTGGCAACGTTTTGCCCGGCAGCGGCTGGCGCGCAGCCGCTGGAATCGCGATCCGGCGGCCC
TGGATCCGGCCGACACGCCGGCTTTTGAACAGGCCTGGCAACGCCAGTGCCATATGGAGCAGACGATCGTCGCGC
GGGTCCCTGAAGGCGATATTCCGGCGGCGTTGCTGGAGAATATCGCTGCCTCCCTTGCCATCTGGCTCGACGAGG
GGGATTTTGCGCCGCCCGAGCGCGCTGCCATCGTGCGCCATCACGCCCGGCTGGAACTCGCCTTCGCCGATATCG
CCCGCCAGGCGCCGCAGCCGGATCTCTCCACGGTACAGGCATGGTATCTGCGCCACCAGACGCAGTTTATGCGCC
CGGAACAGCGTCTGACCCGCCATTTACTGCTGACGGTCGATAACGACCGCGAAGCCGTGCACCAGCGGATCCTCG
GCCTGTATCGGCAAATCAACGCCTCGCGGGACGCTTTCGCGCCGCTGGCCCAGCGCCATTCCCACTGCCCGAGCG
CGCTGGAAGAGGGTCGTTTAGGCTGGATTAGCCGTGGCCTGCTCTATCCGCAGCTCGAGACCGCGCTGTTTTCAC
TGGCGGAAAACGCGCTAAGCCTTCCCATCGCCAGCGAACTGGGCTGGCATCTTTTATGGTGCGAAGCGATTCGCC
CCGCCGCGCCCATGGAGCCGCAGCAGGCGCTGGAGAGCGCGCGCGATTATCTTTGGCAGCAGAGCCAGCAGCGCC
ATCAGCGCCAGTGGCTGGAACAGATGATTTCCCGTCAGCCGGGACTGTGCGGGTAGCCTCGGCGGCTACCCGTTA
ACGCCTACAGCACGGTGCGTTTAATCTCCTCAAGCCAGCTCGCCAGACGCGCTTCGGTCTGGTCGAACTGGTTAT
CCTGATCCAGCACCAGCCCAACAAAGCGGTCGCCTTCCAGCGCCGAGGACGCGCTGAATTCATAACCCTCATTTG
GCCAGCTGCCAATCATCTGCGCGCCGCGCGCGCTCAGGGCGTCGAACAGCGGGCGCATCCCGCTGACGAAGTTGT
CCGGATAGCCTCTCTGATCGCCGAGGCCGAACAGCGCCACGGTTTTCCCTTTCAGGCTGGCGTCGTCGAGGCCGC
TGATAAATTCGCTCCATGACTCGCTTTCGCATCCGGCCTCCAGCCCCGGCAGCTGGCCGTCGCCGAGCGTCGGCG
TGCCCAGCAGCAGCACCGGATAGGCCATAAAGTCGTCCAGCGTCGTGCGGTTAATGTTGACCGGGGCATCCGCCA
GCTCGCCCAGTTGCTTATGGATCATTTTCGCGATTTTGCGGGTTTTACCGGTATCGGTGCCAAAGAAAATACCAA
TGTTCGCCATGTTGCGCTCCTGTCGGAAAAGGGGGTTGAAAATACGCGTTCTCGCAGGGGTATTGCGAAGGCTGT
GCCAGGTTGCTTTGCACTACCGCGGCCCATCCCTGCCCCAAAACGATCGCTTCAGCCCTCTCCCGCCGCGCGCGG
CGGGGCTGGCGGGGCGCTTAAAATGCAAAAAGCGCCTGCTTTTCCCCTACCGGATCAATGTTTCTGCACATCACG
CCGATAAGGGCGCACGGTTTGCATGGTTATCACCGTTCGGAAAACACCGCGGCGTCCCTGTCACGGTGTCGGACA
AATTGTCATAACTGCGACACAGGAGTTTGCGATGACCCTGAATATGATGCTCGATAACGCCGTACCCGAGGCGAT
TGCCGGTGCGCTGACTCAACAACATCCGGGGCTGTTTTTTACAATGGTCGAACAGGCATCGGTAGCGATTTCCCT
CACCGATGCCCGGGCGAATATTACCTACGCCAACCCGGCGTTTTGCCGCCAGACTGGATACTCGCTGGCGCAATT
GCTCAATCAAAACCCGCGCCTGCTGGCCAGCAGCCAGACGCCGCGCGAGATCTACCAGGAGATGTGGCAAACCCT
GCTCCAGCGCCAGCCGTGGCGCGGTCAGCTAATTAATCAGGCCCGCGACGGCGGCCTGTATCTGGTAGATATCGA
TATCACGCCGGTGCTGAATCCGCAGGGCGAGCTGGAGCATTATCTGGCGATGCAGCGGGATATCAGCGTCAGCTA
TACCCTGGAACAGCGGCTGCGCAATCATATGACGCTAATGGAAGCGGTGCTCAATAACATCCCCGCCGCCGTGGT
CGTGGTCGATGAGCAGGATCGGGTGGTGATGGATAATCTCGCCTACAAAACGTTCTGCGCGGACTGCGGCGGGAA
AGAGCTGCTGGTCGAGCTCCAGGTTTCCCCGCGCAAAATGGGGCCCGGCGCGGAGCAAATCCTGCCGGTGGTGGT
TCGCGGCGCGGTCCGCTGGCTGTCGGTAACCTGCTGGGCGCTGCCCGGCGTGAGTGAAGAAGCCAGCCGCTACTT
CGTCGACAGCGCCCCGGCGCGCACGCTGATGGTGATCGCCGACTGTACCCAGCAGCGCCAGCAGCAGGAGCAGGG
CCGGCTCGACCGTCTGAAACAGCAAATGACCGCCGGTAAGCTGCTGGCCGCGATTCGCGAGTCGCTGGACGCGGC
GCTGATTCAGCTTAATTGCCCAATCAATATGCTGGCGGCGGCCCGCCGGCTGAACGGCGAAGGCAGCGGCAACGT
GGCGCTGGACGCGGCGTGGCGCGAAGGTGAAGAGGCCATGGCGCGCCTGCAGCGCTGCCGCCCTTCTCTTGAGCT
GGAAAGCAATGCCGTCTGGCCGCTTCAGCCCTTTTTTGACGACCTGTACGCCCTCTACCGCACCCGCTTTGACGA
TCGCGCGCGGCTGCAGGTGGACATGGCATCGCCGCATCTGGTCGGCTTCGGCCAGCGTACCCAGCTGCTGGCCTG
CTTGAGTTTATGGCTCGACCGGACGCTGGCCCTCGCCGCCGAGCTGCCCTCCGTACCGCTGGAGATCGAGCTTTA
CGCCGAAGAGGACGAGGGCTGGCTCTCTTTGTATCTCAACGACAATGTCCCGCTGCTGCAGGTGCGCTACGCCCA
CTCCCCCGATGCCCTAAACTCTCCCGGCAAAGGGATGGAGCTGCGGCTGATCCAAACGCTGGTCGCCTACCACCG
CGGCGCGATTGAACTGGCTTCGCGACCGCAGGGAGGCACCAGCCTGGTTCTGCGTTTCCCGCTCTTTAATACCCT
GACCGGAGGTGAGCAATGATCCATAAATCCGATTCGGACACCACCGTCAGACGTTTCGATCTCTCCCAGCAGTTT
ACCGCCATGCAGCGGATAAGCGTGGTCCTGAGTCGCGCCACCGAAGCGAGCAAAACCCTGCAGGAGGTTCTGAGC
GTGCTACATAACGATGCCTTTATGCAGCACGGGATGATTTGCCTGTACGACAGCCAGCAGGAGATCCTGAGCATC
GAAGCGCTGCAGCAAACGGAAGATCAGACGCTGCCCGGCAGTACGCAAATTCGCTACCGGCCGGGGGAAGGATTA
GTCGGTACCGTGCTGGCGCAGGGCCAGTCGCTGGTGCTGCCGCGCGTCGCCGACGACCAGCGTTTTCTCGATCGT
CTGAGCCTGTACGACTATGACCTGCCGTTTATCGCCGTTCCGCTGATGGGCCCCCACTCCCGGCCCATCGGCGTA
CTGGCGGCGCACGCGATGGCGCGTCAGGAAGAGCGGCTGCCCGCCTGCACGCGCTTTCTCGAAACCGTCGCCAAT
CTGATCGCCCAGACGATTCGCCTGATGATCCTGCCAACCTCCGCCGCGCAGGCGCCGCAGCAGAGCCCCAGAATA
GAGCGCCCGCGCGCCTGTACCCCTTCGCGCGGTTTCGGCCTGGAAAATATGGTCGGTAAAAGCCCGGCGATGCGG
CAGATTATGGATATTATTCGTCAGGTTTCCCGCTGGGATACCACGGTGCTGGTACGCGGCGAGAGCGGCACCGGG
AAAGAGCTCATCGCCAACGCCATCCACCATAATTCTCCGCGCGCCGCCGCGGCGTTCGTCAAATTTAACTGCGCG
GCGCTGCCGGACAACCTGCTGGAGAGCGAGCTGTTTGGTCATGAGAAAGGCGCGTTTACCGGCGCGGTGCGCCAG
CGGAAAGGCCGCTTTGAGCTGGCGGACGGCGGCACCTTATTCCTCGATGAGATCGGCGAAAGCAGCGCCTCGTTT
CAGGCTAAGCTACTGCGTATTCTGCAAGAGGGGGAGATGGAGCGCGTCGGCGGCGACGAAACCCTGCGGGTCAAC
GTGCGCATTATCGCGGCGACCAACCGCCATCTGGAAGAGGAGGTGCGGCTGGGTCATTTCCGCGAGGATCTATAC
TACCGCCTGAACGTAATGCCTATCGCGCTGCCGCCGCTGCGCGAGCGCCAGGAGGATATCGCCGAGCTGGCGCAC
TTTCTGGTGCGAAAAATCGCCCACAGCCAGGGGCGAACGCTGCGCATCAGCGATGGGGCGATTCGCCTGCTGATG
GAGTACAGCTGGCCGGGAAACGTGCGCGAACTGGAAAACTGTCTCGAACGTTCGGCGGTGCTGTCGGAAAGCGGC
CTGATAGACCGGGACGTGATTCTGTTCAACCATCGCGATAACCCGCCGAAAGCGCTCGCCAGCAGCGGCCCGGCG
GAGGACGGCTGGCTCGATAACAGCCTCGACGAGCGCCAGCGGCTGATCGCCGCCCTGGAAAAAGCGGGCTGGGTG
CAGGCCAAAGCGGCGCGGCTGCTCGGCATGACCCCGCGCCAGGTGGCGTATCGCATTCAGATTATGGATATCACC
ATGCCGCGACTGTGAAGCCTTATGTGAGATTCAGGACATTGTCGCCAGCGCGGCGGAATTGCGACAATTCAGGGA
CGCGGGTTGCCGGTTAAAAAGTCTACTTTTCATGCGGTTGCGAAATTAACCTCTGGTACAGCATTTGCAGCAGGA
AGGTATCGCCCAACCACGAAGGTACGACCATGACTTCCTGCTCCTCTTTTTCTGGCGGCAAAGCCTGCCGCCCGG
CGGATGACAGCGCATTGACGCCGCTTGTGGCCGATAAAGCTGCCGCGCACCCCTGCTACTCTCGCCATGGGCATC
ACCGTTTCGCGCGGATGCATCTGCCCGTCGCGCCCGCCTGCAATTTGCAGTGCAACTACTGTAATCGCAAATTCG
ATTGCAGCAACGAGTCCCGCCCCGGGGTATCGTCAACGCTGCTGACGCCTGAACAGGCGGTCGTGAAAGTGCGTC
AGGTCGCGCAGGCGATCCCGCAGCTTTCGGTGGTGGGCATCGCCGGGCCCGGCGATCCGCTCGCCAATATCGCCC
GCACCTTTCGCACCCTGGAGCTGATCCGCGAACAGCTGCCGGACCTGAAATTATGCCTGTCGACCAACGGACTGG
TGCTGCCTGACGCGGTGGACCGCCTGCTGGATGTCGGCGTTGACCACGTCACGGTCACCATTAACACCCTCGACG
CGGAGATTGCCGCGCAAATCTACGCCTGGCTATGGCTGGACGGCGAACGCTACAGCGGGCGCGAAGCGGGAGAGA
TCCTGATTGCCCGTCAGCTTGAGGGCGTACGCAGGCTGACCGCCAAAGGCGTGCTGGTGAAAATAAATTCGGTGC
TGATCCCCGGTATCAACGATAGCGGCATGGCCGGCGTGAGCCGCGCGCTGCGGGCCAGCGGCGCGTTTATCCATA
ATATTATGCCGCTGATCGCCAGGCCGGAGCACGGCACGGTGTTTGGCCTCAACGGCCAGCCGGAGCCGGACGCCG
AGACGCTCGCCGCCACCCGCAGCCGGTGCGGCGAAGTGATGCCGCAGATGACCCACTGCCACCAGTGTCGCGCCG
ACGCCATTGGGATGCTCGGCGAAGACCGCAGCCAGCAGTTTACCCAGCTTCCGGCGCCAGAGAGTCTCCCGGCCT
GGCTGCCGATCCTCCACCAGCGCGCGCAGCTGCACGCCAGCATTGCGACCCGCGGCGAATCTGAAGCCGATGACG
CCTGCCTGGTCGCCGTGGCGTCAAGCCGCGGGGACGTCATTGATTGTCACTTTGGTCACGCCGACCGGTTCTACA
TTTACAGCCTCTCGGCCGCCGGTATGGTGCTGGTCAACGAGCGCTTTACGCCCAAATATTGTCAGGGGCGCGATG
ACTGCGAGCCGCAGGATAACGCAGCCCGGTTTGCGGCGATCCTCGAACTGCTGGCGGACGTTAAAGCCGTATTCT
GCGTGCGTATCGGCCATACGCCGTGGCAACAGCTGGAACAGGAAGGCATTGAACCCTGCGTTGACGGCGCGTGGC
GGCCGGTCTCCGAAGTGCTGCCCGCGTGGTGGCAACAGCGTCGGGGGAGCTGGCCTGCCGCGTTGCCGCATAAGG
GGGTCGCCTGATGCCGCCGCTCGACTGGTTGCGGCGCTTATGGCTGCTGTACCACGCGGGGAAAGGCAGCTTTCC
GCTGCGCATGGGGCTTAGCCCGCGCGATTGGCAGGCGCTGCGGCGGCGCCTGGGCGAGGTGGAAACGCCGCTCGA
CGGCGAGACGCTCACCCGTCGCCGCCTGATGGCGGAGCTCAACGCCACCCGCGAAGAGGAGCGCCAGCAGCTGGG
CGCCTGGCTGGCGGGCTGGATGCAGCAGGATGCCGGGCCGATGGCGCAGATTATCGCCGAGGTTTCGCTGGCGTT
TAACCATCTCTGGCAGGATCTTGGTCTGGCATCGCGCGCCGAATTGCGCCTGCTGATGAGCGACTGCTTTCCACA
GCTGGTGGTGATGAACGAACACAATATGCGCTGGAAAAAGTTCTTTTATCGTCAGCGCTGTTTGCTGCAACAGGG
GGAAGTTATCTGCCGTTCGCCAAGCTGCGACGAGTGCTGGGAACGCAGCGCCTGTTTTGAGTAGCCGTTTCCCGA
AGGGGGCGCTGCAAACAAAAAGCCGGAGGTTTCCCTCCGGCTTTTCACATCATCAAATGTGATTATGCGACGTCT
TCGTACTGCGGCACCGGGTTGCGGAAGCTTTTGGTCACGCAGGCCTCCGTAGACCAGACCAATACCGCCCCAGAT
CAGGCCGAGAACCATGGAGCTCTCTTCGAGGTTAATCCACAGTGCGCCGACGGTCAGCGCGCCGCAGACCGGCAG
AATCAGATAGTTGAAGTGGTCTTTCAGCGTTTTGTTGCGCTTTTCACGGATCCAGAACTGGGAGATCACCGACAG
GTTAACGAAGGTGAACGCCACCAGCGCGCCGAGGTTAATCGGCGCCGTCGCCGTGACGAGGTCGAGTTTAATCGC
CAGCAGCGCGATCGCGCAACCAGCAGCACGTTCCATGCCGGAGTACGCCGTTTCGGATGCACGTAGCCGAAGAAA
CGCGTCGGGAACACGCCGTCGCGGCCCATCACGTACATCAGACGGGAAACGCCCGCGTGCGCGGCCGTGCCGGAT
GCCAGTACGGTAACGCTGGAGAAAATCAGCACGCCCCACTGGAAGGTTTTGCCCGCCACGTACAGCATGATTTCA
GGCTGCGAGGCGTCCGGATCTTTGAAGCGCGAGATGTCCGGGAAGTACAGCTGCAG
SEQ ID NO: 10, Azotobacter vinelandii nifHDK gene cluster (Gene Bank AVINIFA)
CCCGGGCCCAGATAGGGAACGATGTCGCCCGAGCCGAGCTGGGCGAGGATTTCCTTTAATAAGCTGTCGGTCACT
GAACTCTCCTGCTGAGGGAAGGGCAAGAATCGACACCTTATTGCAATAAGTGTGCCAAGATTTCGTTGTTTAACT
AATTGAATTTAAAAGAAATCATTGGTGATTTCGGAATGGCTTGTCGTATCCGTGGGCCAGGATGGGGCGTGGCTT
CACGACAATTGTCAGTTTTGTCACAGGGGGCCGGACCAGGATGGTGGACGCTCGATGGGGATGTCGGGCCATTGT
TCGGTTGTAGCAATTACACACATGTCGGAGTAGGGGGATTGTGAGGGGGATTGTTGTGTATCACCCCCTGCAGCT
CCCGTCGATGGATAATTAATCATTTAAAATCAATGGTTTATTTATGTGTTGCGGGTGCTGGCACAGACGCTGCAT
TACCTTTGGTGCGCGGAGTTGTTCGGGCTTACGGCCGAACGTTCAAGTGGAAATGCAACCTGAGGAAATTAACTA
TGGCTATGCGTCAATGCGCCATCTACGGCAAAGGTGGTATCGGTAAGTCCACCACTACTCAGAACCTGGTGGCAG
CCCTGGCTGAGATGGGCAAGAAGGTCATGATCGTTGGTTGTGACCCGAAAGCTGACTCCACCCGCCTGATCCTGC
ACTCCAAGGCCCAGAACACCATCATGGAAATGGCTGCCGAAGCCGGTACCGTGGAAGATCTGGAGCTGGAAGACG
TGCTGAAGGCTGGCTACGGCGGCGTCAAGTGCGTTGAGTCCGGTGGTCCGGAGCCGGGCGTTGGCTGCGCCGGCC
GTGGTGTTATCACAGCAATCAACTTCCTGGAAGAGGAAGGCGCCTACGAAGACGATCTGGACTTCGTATTCTACG
ACGTCCTGGGCGACGTGGTGTGTGGCGGCTTCGCCATGCCGATCCGCGAGAACAAGCCCCAAGAAATCTACATCG
TCTGCTCCGGTGAGATGATGGCCATGTACGCCGCCAACAACATCTCCAAGGGCATCGTGAAGTATGCCAACTCCG
GCAGCGTGCGTCTGGGCGGCCTGATCTGCAACAGCCGTAACACCGACCGCGAAGACGAGCTGATCATCGCTCTGG
CCAACAAGCTGGGCACCCAGATGATCCACTTCGTGCCGCGTGACAACGTCGTGCAGCGCGCCGAAATCCGCCGCA
TGACCGTGATCGAATACGATCCGAAAGCCAAGCAAGCCGACGAATACCGCGCTCTGGCCCGCAAGGTCGTCGACA
ACAAACTGCTGGTCATCCCGAACCCGATCACCATGGACGAGCTCGAAGAGCTGCTGATGGAATTCGGTATCATGG
AAGTCGAAGACGAATCCATCGTCGGCAAAACCGCCGAAGAAGTCTGATAGCCGCTCCGGTTTCAGAAGGACTTTA
CAGGGCAGATTGGCTCTGTCGGGGTGGCGCCCCCCGCATTGGGCGGGCGCCCACCCGTTACCCGCATTATGAACG
CTAAGGCAAGAGGAGTCATACCCATGACCCGTATGTCGCGCGAAGAGGTTGAATCCCTCATCCAGGAAGTTCTGG
AAGTTTATCCCGAGAAGGCTCGCAAGGATCGTAACAAGCACCTGGCCGTCAACGACCCGGCGGTTACCCAGTCCA
AGAAGTGCATCATCTCCAACAAGAAGTCCCAGCCCGGTCTGATGACCATCCGCGGCTGCGCCTACGCCGGTTCCA
AAGGCGTGGTCTGGGGCCCCATCAAGGACATGATCCACATCTCCCACGGTCCGGTAGGCTGCGGCCAGTATTCGC
GCGCCGGCCGTCGTAACTACTACATCGGTACCACCGGTGTGAACGCCTTCGTCACCATGAACTTCACCTCGGACT
TCCAGGAGAAGGACATCGTGTTCGGTGGCGACAAGAAGCTCGCCAAACTGATCGACGAAGTGGAAACCCTGTTCC
CGCTGAACAAGGGTATCTCCGTCCAGTCCGAGTGCCCGATCGGCCTGATCGGCGACGACATCGAATCCGTGTCCA
AGGTCAAGGGCGCCGAGCTCAGCAAGACCATCGTACCGGTCCGTTGCGAAGGCTTCCGCGGCGTTTGCCAGTCCC
TGGGCCACCACATCGCCAACGACGCAGTCCGCGACTGGGTCCTGGGCAAGCGTGACGCCGACACCACCTTCGCCA
GCACTCCTTACGATGTGGCCATCATCGGCGACTACAACATCGGCGGCGACGCCTGGTCTTCCCGCATCCTGCTGG
AAGAAATGGGCCTGCGTTGCGTAGCCCAGTGGTCCGGCGACGGCTACATCTCCCAAATCGAGCTGACCCCGAAGG
TCAAGCTGAACCTGGTTCACTGCTACCGCTCGATGAACTACATCTCCCGTCACATGGAAGAGAAGTACGGTATCC
CATGGATGGAGTACAACTTCTTCGGCCCGACCAAGACCATCGAGTCGCTGCGTGCCATCGCCGCCAAGTTCGACG
AGAGCATCCAGAAGAAGTGCGAAGAGGTCATCGCCAAGTACAAGCCCGAGTGGGAAGCGGTGGTCGCCAAGTACC
GTCCGCGCCTGGAAGGCAAGCGCGTCATGCTCTACATCGGTGGCCTGCGTCCGCGCCACGTGATCGGCGCCTACG
AAGACCTGGGCATGGAAGTGGTGGGTACCGGCTACGAGTTCGCCCACAACGACGACTATGACCGGACCATGAAAG
AAATGGGTGACTCCACCCTGCTGTACGATGACGTGACCGGCATGGAATTCGAAGAATTCGTCAAGCGCATCAAGC
CCGACCTGATCGGCTCCGGTATCAAGGAGAAGTTCATCTTCCAGAAGATGGGCATCCCCTTCCGTCAAATGCACT
CCTGGGATTATTCCGGCCCCTACCACGGCTTCGATGGCTTCGCCATCTTCGCCCGTGACATGGACATGACCCTGA
ACAATCCGTGCTGGAAGAAACTGCAGGCTCCCTGGGAAGCTTCCGAAGGCGCCGAGAAAGTCGCCGCCAGCGCCT
GATAGCAGAGCAATCGTACGCAACGTCCGCTGCGGGCGGTTTCCGCCGGCCGACATTCCGCTAACGCCGTTCACA
GATGAGTGAGGCGTAGGAGAGAGTCATGAGCCAGCAAGTCGATAAAATCAAAGCCAGCTACCCGCTGTTCCTCGA
TCAGGACTACAAGGACATGCTTGCCAAGAAGCGCGACGGCTTCGAGGAAAAGTATCCGCAGGACAAGATCGACGA
AGTATTCCAGTGGACCACCACCAAGGAATACCAGGAGCTGAACTTCCAGCGCGAAGCCCTGACCGTCAACCCGGC
CAAGGCTTGCCAGCCGCTGGGCGCCGTTCTCTGCGCCCTCGGTTTCGAGAAGACCATGCCCTACGTGCACGGTTC
CCAGGGTTGCGTCGCCTACTTCCGCTCCTACTTGAACCGTCATTTCCGCGAGCCGGTTTCCTGCGTTTCCGACTC
CATGACCGAAGACGCGGCAGTGTTCGGCGGCCAGCAGAACATGAAGGACGGTCTGCAGAACTGTAAGGCTACCTA
CAAGCCCGACATGATCGCAGTGTCCACCACCTGCATGGCCGAGGTCATCGGTGACGACCTCAACGCCTTCATCAA
CAACTCGAAGAAGGAAGGTTTCATTCCTGACGAGTTCCCGGTGCCGTTCGCCCATACCCCGAGCTTCGTGGGCAG
CCACGTGACCGGCTGGGACAACATGTTCGAAGGCATTGCTCGCTACTTCACCCTGAAGTCCATGGACGACAAGGT
GGTTGGCAGCAACAAGAAGATCAACATCGTCCCCGGCTTCGAGACCTACCTGGGCAACTTCCGCGTGATCAAGCG
CATGCTTTCGGAAATGGGCGTGGGCTACAGCCTGCTCTCCGATCCGGAAGAAGTGCTGGACACCCCGGCTGACGG
CCAGTTCCGCATGTACGCGGGCGGCACCACTCAGGAAGAGATGAAGGACGCTCCGAACGCCCTCAACACCGTCCT
GCTGCAGCCGTGGCACCTNGAGAAGACCAAGAAGTTCGTCGAGGGTACCTGGAAGCACGAAGTACCGAAGCTGAA
CATCCCGATGGGCCTGGACTGGACCGACGAGTTCCTGATGAAAGTCAGCGAAATCAGCGGCCAGCCGATTCCGGC
GAGCCTGACCAAGGAGCGTGGCCGTCTGGTCGACATGATGACCGACTCCCACACCTGGCTGCACGGCAAGCGTTT
CGCCCTGTGGGGTGATCCGGACTTCGTGATGGGCCTGGTCAAGTTCCTGCTGGAACTGGGTTGCGAGCCGGTACA
CATTCTCTGCCACAACGGCAACAAGCGTTGGAAGAAGGCGGTCGACGCCATCCTCGCCGCTTCGCCCTACGGCAA
GAATGCTACCGTCTACATCGGCAAGGACCTGTGGCACCTGCGTTCGCTGGTCTTCACCGACAAGCCGGACTTCAT
GATCGGCAACAGCTACGGTAAGTTCATCCAGCGCGACACCCTGCACAAGGGCAAGGAGTTCGAGGTTCCGCTGAT
CCGTATCGGCTTCCCGATCTTCGACCGTCATCACCTGCATCGCTCCACCACCCTGGGTTACGAGGGCGCCATGCA
GATCCTGACCACCCTGGTGAACTCGATCCTGGAACGTCTGGACGAGGAAACCCGCGGTATGCAGGCCACCGACTA
CAACCACGACCTGGTACGCTAAGTCGTCGGTTCAAGTGGTATCGGCCGGAGCGGCGCAAGCTGCTCTCCCTTGGC
GGCGGCCGCAGGTGGTCGGGCCTTTTGCCCGCGATCTGCGGCAACCGCCAAACCCGTCTAAGGAGCAAGCCCATG
CCCAGCGTCATGATTCGCCGCAACGACGAAGGCCAACTGACCTTCTATATCGCCAAGAAAGACCAGGAAGAGATC
GTGGTGTCCCTGGAGCATGACAGCCCCGAACTCTGGGGTGGCGAAGTCACCCTCGGCGACGGTTCGACCTATTTC
ATCGAGCCGATACCGCAACCCAAGCTGCCGATC
SEQ ID NO: 11, Nicotiana tabacum chloroplast Prrn promoter (GeneBank BD174938)
GCTCTAGTTGGATTTGCTCCCCCGCCGTCGTTCAATGAGAATGGATAAGAGGCTCGTGGGATTGACGTGAGGGGG
CAGGGATGGCTATATTTCTGGGAGCGAACTCCGGGCGAATTTGAAGCGCTTGGATACAGTTGTAGGGAGGGATCC
SEQ ID NO: 12, Cauliflower Mosaic Virus 35S promoter (GeneBank S51061)
TCCACTGACGTAAGGGATGACGCACAATCCCACTATCCTTCGCAAGACCCTTCCTCTATATAAGGAAGTTCATTT
CATTTGGAGAGGACACGCTGAAATCACCAGTCTCTCTCTACAAATCTATCTCTCTCTATTTTCTCCATAATAATG
TGTGAGTAGTTCCCAGATAAGGGAATTAGGGTTCTTATAGGGTTTCGCTCACGTGTTGAG
SEQ ID NO: 13, Nicotiana tabacum chloroplast psbA terminator (GeneBank DQ489715)
GATCCTGGCCTAGTCTATAGGAGGTTTTGAAAAGAAAGGAGCAATAATCATTTTCTTGTTCTATCAAGAGGGTGC
TATTGCTCCTTTCTTTTTTTCTTTTTATTTATTTACTAGTATTTTACTTACATAGACTTTTTTGTTTACATTATA
GAAAAAGAAGGAGAGGTTATTTTCTTGCATTTATTCATGATTGAGTATTCTATTTTGATTTTGTATTTGTTTAAA
ATTGTAGAAATAGAACTTGTTTCTCTTCTTGCTAATGTTACTATATCTTTTTGATTTTTTTTTTCCAAAAAAAAA
ATCAAATTTTGACTTCTTCTTATCTCTTATCTTTGAATATCTCTTATCTTTGAAATAATAATATCATTGAAATAA
GAAAGAAGAGCTATATTCGA
SEQ ID NO: 14, Cauliflower Mosaic Virus 35S terminator (GeneBank AY818367)
GTCCGCAAAAATCACCAGTCTCTCTCTACAAATCTATCTCTCTCTATTTTTCTCCAGAATAATGTGTGAGTAGTT
CCCAGATAAGGGAATTAGGGTTCTTATAGGGTTTCGCTCATGTGTTGAGCATATAAGAAACCCTTAGTATGTATT
TGTATTTGTAAAATACTTCTATCAATAAAATTTCTAATTCCTAAAACCAAAATCCAGTGA
SEQ ID NO: 15, Spectinomycin resistance gene aadA (GeneBank DQ211347)
ATGGGGGAAGCGGTGATCGCCGAAGTATCGACTCAACTATCAGAGGTAGTTGGCGTCATCGAGCGCCATCTCGAA
CCGACGTTGCTGGCCGTACATTTGTACGGCTCCGCAGTGGATGGCGGCCTGAAGCCACACAGTGATATTGATTTG
CTGGTTACGGTGACCGTAAGGCTTGATGAAACAACGCGGCGAGCTTTGATCAACGACCTTTTGGAAACTTCGGCT
TCCCCTGGAGAGAGCGAGATTCTCCGCGCTGTAGAAGTCACCATTGTTGTGCACGACGACATCATTCCGTGGCGT
TATCCAGCTAAGCGCGAACTGCAATTTGGAGAATGGCAGCGCAATGACATTCTTGCAGGTATCTTCGAGCCAGCC
ACGATCGACATTGATCTGGCTATCTTGCTGACAAAAGCAAGAGAACATAGCGTTGCCTTGGTAGGTCCAGCGGCG
GAGGAACTCTTTGATCCGGTTCTTGAACAGGATCTATTTGAGGCGCTAAATGAAACCTTAACGCTATGGAACTCG
CCGCCCGACTGGGCTGGCGATGAGCGAAATGTAGTGCTTACGTTGTCCCGCATTTGGTACAGCGCAGTAACCGGC
AAAATCGCGCCGAAGGATGTCGCTGCCGACTGGGCAATGGAGCGCCTGCCGGCCCAGTATCAGCCCGTCATACTT
GAAGCTAGACAGGCTTATCTTGGACAAGAAGAAGATCGCTTGGCCTCGCGCGCAGATCAGTTGGAAGAATTTGTC
CACTACGTGAAAGGCGAGATCACCAAGGTAGTCGGCAAATAA
SEQ ID NO: 16, pUC19 (GeneBank L09137)
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTATG
CGGCATCAGAGCAGATTGTACTGAGAGTGCACCATATGCGGTGTGAAATACCGCACAGATGCGTAAGGAGAAAAT
ACCGCATCAGGCGCCATTCGCCATTCAGGCTGCGCAACTGTTGGGAAGGGCGATCGGTGCGGGCCTCTTCGCTAT
TACGCCAGCTGGCGAAAGGGGGATGTGCTGCAAGGCGATTAAGTTGGGTAACGCCAGGGTTTTCCCAGTCACGAC
GTTGTAAAACGACGGCCAGTGAATTCGAGCTCGGTACCCGGGGATCCTCTAGAGTCGACCTGCAGGCATGCAAGC
TTGGCGTAATCATGGTCATAGCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTAATTGCGTTGCGCTCACTGCCC
GCTTTCCAGTCGGGAAACCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTCCAACCCGGTAAGACACGAC
TTATCGCCACTGGCAGCAGCCACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTCTACGGGGTCTGACGCTCAGTGGAAC
GAAAACTCACGTTAAGGGATTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCAATGCTTAATCAGTGAGGCA
CCTATCTCAGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGCGTCAATACGGGAT
AATACCGCGCCACATAGCAGAACTTTAAAAGTGCTCATCATTGGAAAACGTTCTTCGGGGCGAAAACTCTCAAGG
ATCTTACCGCTGTTGAGATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATAAGGGCGACACGGAAATGT
TGAATACTCATACTCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCCACCTGACGTCTAA
GAAACCATTATTATCATGACATTAACCTATAAAAATAGGCGTATCACGAGGCCCTTTCGTC
SEQ ID NO: 17, Nicotiana tabacum chloroplast psbA promoter (GeneBank DQ463359)
GGGCAACCCACTAGCATATCGAAATTCTAATTTTCTGTAGAGAAGTCCGTATTTTTCCAATCAACTTCATTAAAA
ATTTGAATAGATCTACATACACCTTGGTTGACACGAGTATATAAGTCATGTTATACTGTTGAATAACAAGCCTTC
CATTTTCTATTTTGATTTGTAGAAAACTAGTGTGCTTGGGAGTCCCTGATGATTAAATAAACCAAGATTTTACC
SEQ ID NO: 18, Nicotiana tabacum TrnI chloroplast genome locus (GeneBank Z00044)
CTTCGGGAACGCGGACACAGGTGGTGCATGGCTGTCGTCAGCTCGTGCCGTAAGGTGTTGGGTTAAGTCCCGCAA
CGAGCGCAACCCTCGTGTTTAGTTGCCATCGTTGAGTTTGGAACCCTGAACAGACTGCCGGTGATAAGCCGGAGG
AAGGTGAGGATGACGTCAAGTCATCATGCCCCTTATGCCCTGGGCGACACACGTGCTACAATGGCCGGGACAAAG
GGTCGCGATCCCGCGAGGGTGAGCTAACCCCAAAAACCCGTCCTCAGTTCGGATTGCAGGCTGCAACTCGCCTGC
ATGAAGCCGGAATCGCTAGTAATCGCCGGTCAGCCATACGGCGGTGAATTCGTTCCCGGGCCTTGTACACACCGC
CCGTCACACTATGGGAGCTGGCCATGCCCGAAGTCGTTACCTTAACCGCAAGGAGGGGGATGCCGAAGGCAGGGC
TAGTGACTGGAGTGAAGTCGTAACAAGGTAGCCGTACTGGAAGGTGCGGCTGGATCACCTCCTTTTCAGGGAGAG
CTAATGCTTGTTGGGTATTTTGGTTTGACACTGCTTCACACCCCCAAAAAAAAGAAGGGAGCTACGTCTGAGTTA
AACTTGGAGATGGAAGTCTTCTTTCCTTTCTCGACGGTGAAGTAAGACCAAGCTCATGAGCTTATTATCCTAGGT
CGGAACAAGTTGATAGGACCCCCTTTTTTACGTCCCCATGTTCCCCCCGTGTGGCGACATGGGGGCGAAAAAAGG
AAAGAGAGGGATGGGGTTTCTCTCGCTTTTGGCATAGCGGGCCCCCAGTGGGAGGCTCGCACGACGGGCTATTAG
CTCAGTGGTAGAGCGCGCCCCTGATAATTGCGTCGTTGTGCCTGGGCTGTGAGGGCTCTCAGCCACATGGATAGT
TCAATGTGCTCATCGGCGCCTGACCCTGAGATGTGGATCATCCAAGGCACATTAGCATGGCGTACTCCTCCTGTT
CGAACCGGGGTTTGAAACCAAACTCCTCCTCAGGAGGATAGATGGGGCGATTCGGGTGAGATCCAATGTAGATCC
AACTTTCGATTCACTCGTGGGATCCGGGCGGTCCGGGGGGGACCACCACGGCTCCTCTCTTCTCGAGAATCCATA
CATCCCTTATCAGTGTATGGACAGCTATCTCTCGAGCACAGGTTTAGCAATGGGAAAATAAAATGGAGCACCTAA
CAACGCATCTTCACAGACCAAGAACTACGAGATCGCCCCTTTCATTCTGGGGTGACGGAGGGATCGTACCATTCG
AGCCGTTTTTTTCTTGACTCGAAATGGGAGCAGGTTTGAAAAAGGATCTTAGAGTGTCTAGGGTTGGGCCAGGAG
GGTCTCTTAACGCCTTCTTTTTTCTTCTCATCGGAGTTATTTCACAAAGACTTGCCAGGGTAAGGAAGAAGGGGG
GAACAAGCACACTTGGAGAGCGCAGTACAACGGAGAGTTGTATGCTGCGTTCGGGAAGGATGAATCGCTCCCGAA
AAGGAATCTATTGATTCTCTCCCAATTGGTTGGACCGTAGGTGCGATGATTTACTTCACGGGCGAGGTCTCTGGT
TCAAGTCCAGGATGGCCCAGCTGCGCCAGGGAAAAGAATAGAAGAAGCATCT
SEQ ID NO: 19, Nicotiana tabacum TrnA chloroplast genome locus (GeneBank Z00044)
ACTACTTCATGCATGCTCCACTTGGCTCGGGGGGATATAGCTCAGTTGGTAGAGCTCCGCTCTTGCAATTGGGTC
GTTGCGATTACGGGTTGGATGTCTAATTGTCCAGGCGGTAATGATAGTATCTTGTACCTGAACCGGTGGCTCACT
TTTTCTAAGTAATGGGGAAGAGGACCGAAACGTGCCACTGAAAGACTCTACTGAGACAAAGATGGGCTGTCAAGA
ACGTAGAGGAGGTAGGATGGGCAGTTGGTCAGATCTAGTATGGATCGTACATGGACGGTAGTTGGAGTCGGCGGC
TCTCCCAGGGTTCCCTCATCTGAGATCTCTGGGGAAGAGGATCAAGTTGGCCCTTGCGAACAGCTTGATGCACTA
TCTCCCTTCAACCCTTTGAGCGAAATGCGGCAAAAGAAAAGGAAGGAAAATCCATGGACCGACCCCATCATCTCC
ACCCCGTAGGAACTACGAGATCACCCCAAGGACGCCTTCGGCATCCAGGGGTCACGGACCGACCATAGAACCCTG
TTCAATAAGTGGAACGCATTAGCTGTCCGCTCTCAGGTTGGGCAGTCAGGGTCGGAGAAGGGCAATGACTCATTC
TTAGTTAGAATGGGATTCCAACTCAGCACCTTTTGAGTGAGATTTTGAGAAGAGTTGCTCTTTGGAGAGCACAGT
ACGATGAAAGTTGTAAGCTGTGTTCGGGGGGGAGTTATTGTCTATCGTTGGCCTCTATGGTAGAATCAGTCGGGG
GACCTGAGAGGCGGTGGTTTACCCTGCGGCGGATGTCAGCGGTTCGAGTCCGCTTATCTCCAACTCGTGAACTTA
GCCGATACAAAGCTTTATGATAGCACCCAATTTTTCCGATTCGGCGGTTCGATCTATGATTTATCATTCATGGAC
GTTGATAAGATCCATCCATTTAGCAGCACCTTAGGATGGCATAGCCTTAAAAGTGAAGGGCGAGGTTCAAACGAG
GAAAGGCTTACGGTGGATACCTAGGCACCCAGAGACGAGGAAGGGCGTAGTAATCGACGAAATGCTTCGGGGAGT
TGAAAATAAGCATAGATCCGGAGATTCCCGAATAGGGCAACCTTTCGAACTGCTGCTGAATCCATGGGCAGGCAA
GAGACAACCTGGCGAACTGAAACATCTTAGTAGCCAGAGGAAAAGAAAGCAAAAGCGATTCCCGTAGTAGCGGCG
AGCGAAATGGGAGCAGCCTAAACCGTGAAAACGGGGTTGTGGGAGAGCAATACAAGCGTCGTGCTGCTAGGCGAA
GCAGCCCGAATGCTGCACCCTAGATGGCGAAAGTCCAGTAGCCGAAAGCATCACTAGCTTATGCTCTGACCCGAG
TAGCATGGGGCACGTGGAATCCCGTGTGAATCAGCAAGGACCACCTTGCAAGGCTAAATACTCCTGGGTGACCGA
TAGCGAAGTAGTACCGTGAGGGAAGGGTGAAAAGAACCCCCATCGGGGAGTGAAATAGAACATGAAACCGTAAGC
TCCCAAGCAGTGGGAGGAGCCAGGGCTCTGACCGCGTGCCTGTTGAAGAATGAGCCGGCGACTCATAGGCAGTGG
CTTGGTTAAGGGAACCCACCGGAGCCGTAGCGAAAGCGAGTCTTCATAGG
SEQ ID NO: 20, Chloroplast transformation vector pCTV
GTTTAAACCGGTCTTCGGGAACGCGGACACAGGTGGTGCATGGCTGTCGTCAGCTCGTGCCGTAAGGTGTTGGGT
TAAGTCCCGCAACGAGCGCAACCCTCGTGTTTAGTTGCCATCGTTGAGTTTGGAACCCTGAACAGACTGCCGGTG
ATAAGCCGGAGGAAGGTGAGGATGACGTCAAGTCATCATGCCCCTTATGCCCTGGGCGACACACGTGCTACAATG
GCCGGGACAAAGGGTCGCGATCCCGCGAGGGTGAGCTAACCCCAAAAACCCGTCCTCAGTTCGGATTGCAGGCTG
CAACTCGCCTGCATGAAGCCGGAATCGCTAGTAATCGCCGGTCAGCCATACGGCGGTGAATTCGTTCCCGGGCCT
TGTACACACCGCCCGTCACACTATGGGAGCTGGCCATGCCCGAAGTCGTTACCTTAACCGCAAGGAGGGGGATGC
CGAAGGCAGGGCTAGTGACTGGAGTGAAGTCGTAACAAGGTAGCCGTACTGGAAGGTGCGGCTGGATCACCTCCT
TTTCAGGGAGAGCTAATGCTTGTTGGGTATTTTGGTTTGACACTGCTTCACACCCCCAAAAAAAAGAAGGGAGCT
ACGTCTGAGTTAAACTTGGAGATGGAAGTCTTCTTTCCTTTCTCGACGGTGAAGTAAGACCAAGCTCATGAGCTT
ATTATCCTAGGTCGGAACAAGTTGATAGGACCCCCTTTTTTACGTCCCCATGTTCCCCCCGTGTGGCGACATGGG
GGCGAAAAAAGGAAAGAGAGGGATGGGGTTTCTCTCGCTTTTGGCATAGCGGGCCCCCAGTGGGAGGCTCGCACG
ACGGGCTATTAGCTCAGTGGTAGAGCGCGCCCCTGATAATTGCGTCGTTGTGCCTGGGCTGTGAGGGCTCTCAGC
CACATGGATAGTTCAATGTGCTCATCGGCGCCTGACCCTGAGATGTGGATCATCCAAGGCACATTAGCATGGCGT
ACTCCTCCTGTTCGAACCGGGGTTTGAAACCAAACTCCTCCTCAGGAGGATAGATGGGGCGATTCGGGTGAGATC
CAATGTAGATCCAACTTTCGATTCACTCGTGGGATCCGGGCGGTCCGGGGGGGACCACCACGGCTCCTCTCTTCT
CGAGAATCCATACATCCCTTATCAGTGTATGGACAGCTATCTCTCGAGCACAGGTTTAGCAATGGGAAAATAAAA
TGGAGCACCTAACAACGCATCTTCACAGACCAAGAACTACGAGATCGCCCCTTTCATTCTGGGGTGACGGAGGGA
TCGTACCATTCGAGCCGTTTTTTTCTTGACTCGAAATGGGAGCAGGTTTGAAAAAGGATCTTAGAGTGTCTAGGG
TTGGGCCAGGAGGGTCTCTTAACGCCTTCTTTTTTCTTCTCATCGGAGTTATTTCACAAAGACTTGCCAGGGTAA
GGAAGAAGGGGGGAACAAGCACACTTGGAGAGCGCAGTACAACGGAGAGTTGTATGCTGCGTTCGGGAAGGATGA
ATCGCTCCCGAAAAGGAATCTATTGATTCTCTCCCAATTGGTTGGACCGTAGGTGCGATGATTTACTTCACGGGC
GAGGTCTCTGGTTCAAGTCCAGGATGGCCCAGCTGCGCCAGGGAAAAGAATAGAAGAAGCATCTGGCGCGCCGCG
AAATTAATACGACTCACTATAGGGAGACCACGCCGTCGTTCAATGAGAATGGATAAGAGGCTCGTGGGATTGACG
TGAGGGGGCAGGGATGGCTATATTTCTGGGAGCGAACTCCGGGCGAATTTGAAGCGCTTGGATACGCATGCAGGA
GGTATTTATGGGGGAAGCGGTGATCGCCGAAGTATCGACTCAACTATCAGAGGTAGTTGGCGTCATCGAGCGCCA
TCTCGAACCGACGTTGCTGGCCGTACATTTGTACGGCTCCGCAGTGGATGGCGGCCTGAAGCCACACAGTGATAT
TGATTTGCTGGTTACGGTGACCGTAAGGCTTGATGAAACAACGCGGCGAGCTTTGATCAACGACCTTTTGGAAAC
TTCGGCTTCCCCTGGAGAGAGCGAGATTCTCCGCGCTGTAGAAGTCACCATTGTTGTGCACGACGACATCATTCC
GTGGCGTTATCCAGCTAAGCGCGAACTGCAATTTGGAGAATGGCAGCGCAATGACATTCTTGCAGGTATCTTCGA
GCCAGCCACGATCGACATTGATCTGGCTATCTTGCTGACAAAAGCAAGAGAACATAGCGTTGCCTTGGTAGGTCC
AGCGGCGGAGGAACTCTTTGATCCGGTTCCTGAACAGGATCTATTTGAGGCGCTAAATGAAACCTTAACGCTATG
GAACTCGCCGCCCGACTGGGCTGGCGATGAGCGAAATGTAGTGCTTACGTTGTCCCGCATTTGGTACAGCGCAGT
AACCGGCAAAATCGCGCCGAAGGATGTCGCTGCCGACTGGGCAATGGAGCGCCTGCCGGCCCAGTATCAGCCCGT
CATACTTGAAGCTAGACAGGCTTATCTTGGACAAGAAGAAGATCGCTTGGCCTCGCGCGCAGATCAGTTGGAAGA
ATTTGTCCACTACGTGAAAGGCGAGATCACCAAGGTAGTCGGCAAATAATAGGATCGTTTATTTACAACGGAATG
GTATACAAAGTCAACAGATCTCAACTCGAGACCTCAATGAATTCATTGGACCGCGGATCAAGGTACCATAGATAT
CATTAGCTAGCACTAACTAGTAGTAGTCGACATCAAGAGCTCATTCCACATATGACTGGAGGATCCACAAGGCCT
ATCAAGGCGCCATTAATTAAAGGCCGGCCAATTTAAATACAAGCTTGATCCTGGCCTAGTCTATAGGAGGTTTTG
AAAAGAAAGGAGCAATAATCATTTTCTTGTTCTATCAAGAGGGTGCTATTGCTCCTTTCTTTTTTTCTTTTTATT
TATTTACTAGTATTTTACTTACATAGACTTTTTTGTTTACATTATAGAAAAAGAAGGAGAGGTTATTTTCTTGCA
TTTATTCATGATTGAGTATTCTATTTTGATTTTGTATTTGTTTAAAATTGTAGAAATAGAACTTGTTTCTCTTCT
TGCTAATGTTACTATATCTTTTTGATTTTTTTTTTCCAAAAAAAAAATCAAATTTTGACTTCTTCTTATCTCTTA
TCTTTGAATATCTCTTATCTTTGAAATAATAATATCATTGAAATAAGAAAGAAGAGCTATATTCGACCTGCAGAC
TACTTCATGCATGCTCCACTTGGCTCGGGGGGATATAGCTCAGTTGGTAGAGCTCCGCTCTTGCAATTGGGTCGT
TGCGATTACGGGTTGGATGTCTAATTGTCCAGGCGGTAATGATAGTATCTTGTACCTGAACCGGTGGCTCACTTT
TTCTAAGTAATGGGGAAGAGGACCGAAACGTGCCACTGAAAGACTCTACTGAGACAAAGATGGGCTGTCAAGAAC
GTAGAGGAGGTAGGATGGGCAGTTGGTCAGATCTAGTATGGATCGTACATGGACGGTAGTTGGAGTCGGCGGCTC
TCCCAGGGTTCCCTCATCTGAGATCTCTGGGGAAGAGGATCAAGTTGGCCCTTGCGAACAGCTTGATGCACTATC
TCCCTTCAACCCTTTGAGCGAAATGCGGCAAAAGAAAAGGAAGGAAAATCCATGGACCGACCCCATCATCTCCAC
CCCGTAGGAACTACGAGATCACCCCAAGGACGCCTTCGGCATCCAGGGGTCACGGACCGACCATAGAACCCTGTT
CAATAAGTGGAACGCATTAGCTGTCCGCTCTCAGGTTGGGCAGTCAGGGTCGGAGAAGGGCAATGACTCATTCTT
AGTTAGAATGGGATTCCAACTCAGCACCTTTTGAGTGAGATTTTGAGAAGAGTTGCTCTTTGGAGAGCACAGTAC
GATGAAAGTTGTAAGCTGTGTTCGGGGGGGAGTTATTGTCTATCGTTGGCCTCTATGGTAGAATCAGTCGGGGGA
CCTGAGAGGCGGTGGTTTACCCTGCGGCGGATGTCAGCGGTTCGAGTCCGCTTATCTCCAACTCGTGAACTTAGC
CGATACAAAGCTTTATGATAGCACCCAATTTTTCCGATTCGGCGGTTCGATCTATGATTTATCATTCATGGACGT
TGATAAGATCCATCCATTTAGCAGCACCTTAGGATGGCATAGCCTTAAAAGTGAAGGGCGAGGTTCAAACGAGGA
AAGGCTTACGGTGGATACCTAGGCACCCAGAGACGAGGAAGGGCGTAGTAATCGACGAAATGCTTCGGGGAGTTG
AAAATAAGCATAGATCCGGAGATTCCCGAATAGGGCAACCTTTCGAACTGCTGCTGAATCCATGGGCAGGCAAGA
GACAACCTGGCGAACTGAAACATCTTAGTAGCCAGAGGAAAAGAAAGCAAAAGCGATTCCCGTAGTAGCGGCGAG
CGAAATGGGAGCAGCCTAAACCGTGAAAACGGGGTTGTGGGAGAGCAATACAAGCGTCGTGCTGCTAGGCGAAGC
AGCCCGAATGCTGCACCCTAGATGGCGAAAGTCCAGTAGCCGAAAGCATCACTAGCTTATGCTCTGACCCGAGTA
GCATGGGGCACGTGGAATCCCGTGTGAATCAGCAAGGACCACCTTGCAAGGCTAAATACTCCTGGGTGACCGATA
GCGAAGTAGTACCGTGAGGGAAGGGTGAAAAGAACCCCCATCGGGGAGTGAAATAGAACATGAAACCGTAAGCTC
CCAAGCAGTGGGAGGAGCCAGGGCTCTGACCGCGTGCCTGTTGAAGAATGAGCCGGCGACTCATAGGCAGTGGCT
TGGTTAAGGGAACCCACCGGAGCCGTAGCGAAAGCGAGTCTTCATAGGGCGGCCGCCCGGGTAATACGGTTATCC
ACAGAATCAGGGGATAACGCAGGAAAGAACATGTGAGCAAAAGGCCAGCAAAAGGCCAGGAACCGTAAAAAGGCC
GCGTTGCTGGCGTTTTTCCATAGGCTCCGCCCCCCTGACGAGCATCACAAAAATCGACGCTCAAGTCAGAGGTGG
CGAAACCCGACAGGACTATAAAGATACCAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTATCGCCACTGGCAGCAGCCACTGGT
AACAGGATTAGCAGAGCGAGGTATGTAGGCGGTGCTACAGAGTTCTTGAAGTGGTGGCCTAACTACGGCTACACT
AGAAGAACAGTATTTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTACCATCTGGCCCCAGTGCT
GCAATGATACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTAAGTAGT
TCGCCAGTTAATAGTTTGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATCACTCATGGTTATGGCAGCACTGCAT
AATTCTCTTACTGTCATGCCATCCGTAAGATGCTTTTCTGTGACTGGTGAGTACTCAACCAAGTCATTCTGAGAA
TAGTGTATGCGGCGACCGAGTTGCTCTTGCCCGGCGTCAATACGGGATAATACCGCGCCACATAGCAGAACTTTA
AAAGTGCTCATCATTGGAAAACGTTCTTCGGGGCGAAAACTCTCAAGGATCTTACCGCTGTTGAGATCCAGTTCG
ATGTAACCCACTCGTGCACCCAACTGATCTTCAGCATCTTTTACTTTCACCAGCGTTTCTGGGTGAGCAAAAACA
GGAAGGCAAAATGCCGCAAAAAAGGGAATAAGGGCGACACGGAAATGTTGAATACTCATACTCTTCCTTTTTCAA
TATTATTGAAGCATTTATCAGGGTTATTGTCTCATGAGCGGATACATATTTGAATGTATTTAGAAAAATAAACAA
ATAGGGGTTCCGCGCACATTTCCCCGAAAAGTGCCACCTGACGTCTAAGAAACCATTATTATCATGAC
SEQ ID NO: 21, Streptomyces thermoautotrophicus sdnL protein (may be optionally referred as St1-L)
MALPQTELRPMGKPILRKEDPRLIRGKGREVDDILLPNMLHLCILRSPYAHARIRRIDTSKAEAAPGVKLVLTGE
DLAKMNLAWMPTLAGDVQMVLATGKVLFQYQEVAAVVAETRAQAEDAIQLIEVDYEPLPVVVDPFKALEPDAPIL
REDKEKKSNHIWHWEAGDREETDAIFREAPVVVKQDVREQRVHPSPLEPCGCVADYNPATGKLVVYVTSQAPHVH
RTAIALTTGFPEHMIQVISPDVGGGEGNKVPLYPGYVVAIVASLKLGVPVKWIETRTENIASTHFARDYHMTAEI
AATEDGKMLALRVKTIADHGAFDATANPTKYPAGLYSIVTGSYDFKAAFVEVDGVHTNKPPGGVAYRCSFRVTEA
SYLIERVVDVLARRLKMDPAELRLRNFIRKEQFPYRSPTGWVYDSGDYEKTFKLALERIGYEELRKEQKEKWARG
EFMGIGISTFTEIVGAGPAHSFDILGIKMFDSAEIRVHPTGKVIARLGVRHQGQGHETTFAQIIAEELGLSVDDV
VVEEGDTDTAPYGLGTYASRSTPTAGAAAALCARRIRDKARKIAAHLLEVNEDDVVWDGAAFSVKGLPGRSVTMK
DVAFAAYTNVPDGIEPGLEASYYYNPPNLTFPYGAYIAVVDIDKGTGAVKVRRFLAVDDCGNVINPMIVEGQVHG
GLTEGFAIAFMQDIPYDADGNCLAPNWMDYLVPTAWDTPQLETDRTVTPSPHHPLGAKGVGESPNVGSPAAFVNA
VLDALSPLGVEHIDMPIYPWKVWKILRDTALRSDSMAIPASFQSARREKPGGGIASGPIKWTTSGRQRGRWMNAR
SLTSG
SEQ ID NO: 22, Streptomyces thermoautotrophicus sdnS protein (may be optionally referred as St1-S)
MKIRVKVNGTLYEADVEPRTLLAYFLREELKLTGTHIGCDTTTCGACTVLLDGKAVKSCTVLAVQANGREVMTVE
GLEKDGQLHPLQVAFWEEHALHCGYCTPGMLMASYALLQENPMPTEEEIRFGLSGNVCRCTGYMNIVKAVQSAAR
RLSGASGEAVGEVATSGTAAD
SEQ ID NO: 23, Streptomyces thermoautotrophicus sdnM protein (may be optionally referred as St1-M)
MFPNAFKYEAPASVDEAVRLLAEYGYDGKVLAGGQSLLPMMKLRVAAPAVLIDINGIDALQGWREVDGKLRVGAM
TRHAELEHAKELRDTYPLFFQTARWIADPLIRNRGTIGGSLAHADPGSDWGAAMIALRAEVEARGPQGSRLIPID
EFFVDTFATALNEDELAVAVHVPTPKGPAASRYMKLERRAGDFAIAALAVHVALGTDGRVSEAGIGICACGPIPL
RAAKAEAALIGRPLTEEVIVEASRLVPEDAEPADDLRGSAEYKRDVLRVFAARALRDIAKELQGKVGIQ
SEQ ID NO: 24, Streptomyces thermoautotrophicus sdnO protein (may be optionally referred as St2-D
subunit, or D subunit)
MFELPPLPYPYDALEPYFDAKTMEIHYNGHHGAYVKNLNAALEKYPAWQNKPIEELLQSLDQLPEDIRTAVRNNG
GGHYNHSFWWPMLKKNEGGQPVGKFAEAINRDFGSFEAFKDAFSKAAAGRFGSGWAWVVVEPDGKLTVTTTPNQD
NPVMEGKTVVFGLDVWEHAYYLKYQNRRPEYIQAFWNVVNWDVVNERYEEALKKFGR
SEQ ID NO: 25, DNA segment containing sdnL gene optimized for expression in chloroplasts (designated
as StNitF1)
GGTACCAGGAGGTATTTATGGCTTTGCCTCAAACTGAACTACGACCTATGGGGAAACCCATATTAAGGAAAGAGG
ACCCACGATTAATCCGAGGTAAGGGTCGTTTTGTTGATGATATATTATTACCAAATATGTTACACTTATGTATTT
TAAGGTCCCCCTATGCTCACGCTAGGATACGACGTATCGATACCTCAAAAGCAGAGGCAGCTCCTGGCGTTAAAT
TAGTTCTTACTGGTGAAGATTTAGCTAAAATGAATCTTGCCTGGATGCCCACTTTGGCTGGCGATGTCCAAATGG
TCTTAGCCACAGGTAAGGTACTTTTTCAATACCAAGAAGTTGCAGCAGTAGTTGCTGAAACTAGAGCGCAGGCAG
AGGATGCTATTCAATTAATAGAAGTAGATTATGAACCTTTGCCTGTGGTAGTAGATCCCTTTAAAGCTCTTGAAC
CAGACGCTCCAATCTTACGTGAAGATAAAGAAAAAAAATCAAATCATATCTGGCATTGGGAGGCCGGTGATAGAG
AAGAAACAGATGCTATATTTCGAGAGGCCCCTGTGGTTGTAAAACAAGATGTACGATTTCAAAGAGTTCATCCCT
CCCCACTTGAACCTTGTGGATGTGTCGCTGATTACAATCCAGCTACTGGAAAACTTGTAGTATATGTTACGTCAC
AAGCGCCACATGTACATAGAACAGCAATTGCATTGACCACAGGATTTCCAGAACACATGATACAGGTTATTAGTC
CGGATGTAGGGGGTGGATTCGGAAATAAAGTTCCTCTTTATCCTGGTTATGTTGTGGCTATTGTAGCATCTTTAA
AATTAGGTGTTCCTGTTAAATGGATTGAGACCAGAACGGAAAATATTGCTTCTACACATTTTGCCAGAGACTATC
ACATGACCGCTGAAATTGCCGCTACGGAAGATGGTAAAATGTTAGCCCTTCGTGTTAAAACAATTGCTGATCATG
GTGCCTTTGACGCTACAGCTAATCCTACCAAATATCCTGCTGGACTTTACTCTATAGTTACAGGAAGTTACGACT
TTAAGGCAGCCTTTGTTGAAGTAGATGGTGTACACACTAACAAACCTCCGGGAGGCGTAGCCTACCGATGCTCCT
TTAGAGTTACAGAAGCGAGTTATTTGATAGAACGAGTGGTTGATGTCTTGGCTAGACGATTAAAAATGGACCCCG
CTGAATTAAGACTAAGGAACTTCATTCGTAAGGAGCAATTTCCTTATAGAAGTCCCACTGGCTGGGTATACGATT
CAGGTGATTATGAAAAAACGTTCAAATTAGCTCTTGAGAGAATAGGGTATGAAGAACTACGTAAAGAGCAAAAAG
AAAAATGGGCTAGAGGAGAATTTATGGGTATCGGCATCAGTACTTTTACAGAAATTGTGGGAGCAGGACCAGCCC
ATTCATTCGATATATTAGGGATAAAAATGTTCGATTCAGCAGAAATCAGAGTGCATCCTACCGGAAAGGTTATTG
CTCGTTTAGGTGTTAGACATCAGGGCCAAGGTCATGAGACAACTTTTGCACAAATTATTGCAGAAGAACTTGGCC
TTTCAGTTGATGATGTTGTAGTAGAGGAGGGTGATACGGATACAGCGCCTTATGGACTTGGAACCTATGCCTCTC
GAAGTACACCAACTGCCGGGGCAGCTGCGGCTTTGTGTGCTCGAAGAATTAGAGATAAAGCAAGAAAAATCGCAG
CTCATCTTCTTGAGGTAAACGAAGACGATGTAGTATGGGATGGCGCAGCTTTTTCTGTGAAAGGTTTACCAGGAC
GTTCTGTCACTATGAAGGATGTAGCATTTGCTGCCTATACCAATGTGCCAGATGGCATCGAACCGGGTCTAGAGG
CTAGTTATTATTATAATCCGCCAAACTTAACTTTTCCTTATGGTGCCTACATAGCAGTCGTTGACATTGATAAAG
GAACTGGAGCGGTTAAAGTACGAAGATTTTTAGCTGTAGATGATTGCGGAAATGTAATAAATCCGATGATAGTAG
AAGGACAAGTCCATGGGGGTTTAACAGAAGGTTTTGCAATAGCGTTTATGCAAGATATACCTTATGATGCAGATG
GGAACTGTCTAGCTCCTAATTGGATGGATTACCTTGTACCAACGGCATGGGATACTCCGCAATTAGAGACAGATA
GAACTGTGACCCCTAGTCCTCATCATCCTTTGGGAGCAAAAGGAGTTGGAGAGTCTCCCAATGTCGGATCTCCCG
CCGCATTCGTAAATGCTGTTCTAGATGCCCTATCTCCACTAGGTGTAGAACATATTGATATGCCTATTTATCCTT
GGAAAGTCTGGAAAATATTACGAGACACCGCCCTTCGTTCTGATTCTATGGCTATTCCAGCTTCTTTCCAAAGTG
CACGACGAGAGAAACCTGGCGGAGGTATTGCATCTGGACCCATTAAGTGGACTACATCTGGACGTCAACGAGGGA
GATGGATGAATGCTCGTTCTTTAACTTCTGGCTAATAGGATCGTTTATTTACAACGGAATGGTATACAAAGTCAA
CAGATCTCAAGCTAGC
SEQ ID NO: 26, DNA segment containing sdnS-sdnM-sdnO genes optimized for expression in
chloroplasts (designated as StNitF2)
GCTAGCAGGAGGTATTTATGAAAATTAGAGTAAAGGTTAACGGAACCTTATATGAAGCTGATGTTGAACCGCGTA
CCTTATTGGCTTATTTCTTACGTGAAGAACTTAAATTAACGGGCACTCATATTGGATGTGATACGACAACTTGCG
GGGCTTGTACTGTACTACTTGATGGAAAAGCGGTTAAATCTTGCACTGTACTAGCCGTACAAGCTAACGGCAGAG
AGGTTATGACAGTGGAAGGACTTGAAAAGGATGGTCAACTTCATCCTTTACAGGTTGCTTTTTGGGAGGAACATG
CCCTACATTGTGGATACTGTACACCCGGTATGTTGATGGCTAGTTATGCTTTGTTACAGGAAAATCCGATGCCGA
CCGAGGAAGAGATTAGATTCGGACTTTCAGGGAATGTTTGTCGATGTACTGGCTATATGAATATAGTCAAAGCTG
TACAATCAGCAGCAAGACGTCTTAGTGGAGCTTCTGGTGAAGCTGTTGGAGAGGTAGCAACTTCTGGCACTGCTG
CTGACTAATAGGATCGTTTATTTACAACGGAATGGTATACAAAGTCAACAGATCTCAAAGGAGGTATTTATGTTT
CCCAATGCCTTCAAATATGAGGCTCCAGCTTCAGTAGATGAAGCAGTACGTCTATTAGCCGAGTATGGATATGAT
GGTAAGGTTTTAGCTGGCGGTCAATCCTTGCTACCTATGATGAAACTACGAGTCGCTGCTCCTGCCGTACTTATT
GATATAAATGGTATTGATGCGTTACAAGGATGGCGTGAAGTTGATGGGAAATTACGTGTCGGAGCCATGACACGT
CATGCGGAATTAGAACATGCAAAAGAGCTTAGGGATACTTATCCTTTGTTCTTCCAAACTGCGCGTTGGATTGCT
GATCCGTTAATCCGAAATAGAGGAACAATTGGAGGAAGTCTAGCTCATGCTGATCCAGGGTCTGACTGGGGGGCA
GCAATGATTGCTTTACGAGCTGAGGTGGAAGCCCGTGGTCCTCAAGGGTCTCGTTTAATTCCCATTGACGAATTT
TTTGTTGATACTTTTGCCACCGCTTTAAATGAGGATGAATTGGCCGTTGCCGTACATGTACCGACACCTAAAGGG
CCTGCTGCATCACGATACATGAAACTAGAACGTCGAGCAGGTGATTTTGCTATAGCCGCTTTGGCAGTACATGTC
GCATTAGGTACAGATGGTCGTGTCTCTGAAGCTGGTATTGGGATATGTGCTTGTGGTCCCATTCCGCTAAGAGCC
GCCAAAGCTGAAGCGGCTTTGATCGGACGTCCCTTAACTGAAGAAGTAATAGTAGAAGCGTCTAGATTGGTTCCA
GAAGATGCTGAACCTGCCGATGACTTACGAGGTTCTGCCGAATATAAACGAGATGTACTTAGGGTATTCGCCGCC
CGAGCTTTAAGAGATATAGCAAAAGAACTTCAGGGCAAGGTTGGAATACAATAATAGGATCGTTTATTTACAACG
GAATGGTATACAAAGTCAACAGATCTCAAAGGAGGTATTTATGTTTGAATTACCACCTTTACCATATCCGTACGA
CGCTTTGGAACCGTATTTCGATGCAAAGACTATGGAAATTCATTATAATGGTCATCACGGTGCATACGTCAAGAA
TCTAAATGCTGCTTTAGAAAAGTATCCTGCCTGGCAAAATAAGCCCATTGAAGAATTATTGCAATCTTTAGATCA
GTTACCGGAAGATATTCGTACTGCTGTTCGAAATAACGGAGGCGGACATTATAACCATAGTTTTTGGTGGCCTAT
GTTGAAAAAGAATGAGGGGGGTCAACCTGTAGGAAAATTTGCCGAAGCTATAAATCGTGATTTTGGTAGTTTTGA
AGCGTTTAAGGATGCTTTTTCCAAAGCCGCAGCTGGGCGTTTTGGATCTGGCTGGGCTTGGGTTGTAGTTGAGCC
GGATGGAAAATTAACGGTCACCACAACTCCCAATCAAGATAATCCTGTTATGGAAGGGAAGACTGTAGTGTTTGG
TTTGGATGTTTGGGAACATGCTTATTATTTAAAATATCAAAATAGACGTCCGGAATACATACAGGCTTTTTGGAA
TGTCGTAAATTGGGATGTAGTAAATGAACGATATGAAGAAGCTCTAAAAAAATTCGGCCGTTAATAGGATCGTTT
ATTTACAACGGAATGGTATACAAAGTCAACAGATCTCAACATATG
SEQ ID NO: 27, pCTV-StNitrogenase vector
GTTTAAACCGGTCTTCGGGAACGCGGACACAGGTGGTGCATGGCTGTCGTCAGCTCGTGCCGTAAGGTGTTGGGT
TAAGTCCCGCAACGAGCGCAACCCTCGTGTTTAGTTGCCATCGTTGAGTTTGGAACCCTGAACAGACTGCCGGTG
ATAAGCCGGAGGAAGGTGAGGATGACGTCAAGTCATCATGCCCCTTATGCCCTGGGCGACACACGTGCTACAATG
GCCGGGACAAAGGGTCGCGATCCCGCGAGGGTGAGCTAACCCCAAAAACCCGTCCTCAGTTCGGATTGCAGGCTG
CAACTCGCCTGCATGAAGCCGGAATCGCTAGTAATCGCCGGTCAGCCATACGGCGGTGAATTCGTTCCCGGGCCT
TGTACACACCGCCCGTCACACTATGGGAGCTGGCCATGCCCGAAGTCGTTACCTTAACCGCAAGGAGGGGGATGC
CGAAGGCAGGGCTAGTGACTGGAGTGAAGTCGTAACAAGGTAGCCGTACTGGAAGGTGCGGCTGGATCACCTCCT
TTTCAGGGAGAGCTAATGCTTGTTGGGTATTTTGGTTTGACACTGCTTCACACCCCCAAAAAAAAGAAGGGAGCT
ACGTCTGAGTTAAACTTGGAGATGGAAGTCTTCTTTCCTTTCTCGACGGTGAAGTAAGACCAAGCTCATGAGCTT
ATTATCCTAGGTCGGAACAAGTTGATAGGACCCCCTTTTTTACGTCCCCATGTTCCCCCCGTGTGGCGACATGGG
GGCGAAAAAAGGAAAGAGAGGGATGGGGTTTCTCTCGCTTTTGGCATAGCGGGCCCCCAGTGGGAGGCTCGCACG
ACGGGCTATTAGCTCAGTGGTAGAGCGCGCCCCTGATAATTGCGTCGTTGTGCCTGGGCTGTGAGGGCTCTCAGC
CACATGGATAGTTCAATGTGCTCATCGGCGCCTGACCCTGAGATGTGGATCATCCAAGGCACATTAGCATGGCGT
ACTCCTCCTGTTCGAACCGGGGTTTGAAACCAAACTCCTCCTCAGGAGGATAGATGGGGCGATTCGGGTGAGATC
CAATGTAGATCCAACTTTCGATTCACTCGTGGGATCCGGGCGGTCCGGGGGGGACCACCACGGCTCCTCTCTTCT
CGAGAATCCATACATCCCTTATCAGTGTATGGACAGCTATCTCTCGAGCACAGGTTTAGCAATGGGAAAATAAAA
TGGAGCACCTAACAACGCATCTTCACAGACCAAGAACTACGAGATCGCCCCTTTCATTCTGGGGTGACGGAGGGA
TCGTACCATTCGAGCCGTTTTTTTCTTGACTCGAAATGGGAGCAGGTTTGAAAAAGGATCTTAGAGTGTCTAGGG
TTGGGCCAGGAGGGTCTCTTAACGCCTTCTTTTTTCTTCTCATCGGAGTTATTTCACAAAGACTTGCCAGGGTAA
GGAAGAAGGGGGGAACAAGCACACTTGGAGAGCGCAGTACAACGGAGAGTTGTATGCTGCGTTCGGGAAGGATGA
ATCGCTCCCGAAAAGGAATCTATTGATTCTCTCCCAATTGGTTGGACCGTAGGTGCGATGATTTACTTCACGGGC
GAGGTCTCTGGTTCAAGTCCAGGATGGCCCAGCTGCGCCAGGGAAAAGAATAGAAGAAGCATCTGGCGCGCCGCG
AAATTAATACGACTCACTATAGGGAGACCACGCCGTCGTTCAATGAGAATGGATAAGAGGCTCGTGGGATTGACG
TGAGGGGGCAGGGATGGCTATATTTCTGGGAGCGAACTCCGGGCGAATTTGAAGCGCTTGGATACGCATGCAGGA
GGTATTTATGGGGGAAGCGGTGATCGCCGAAGTATCGACTCAACTATCAGAGGTAGTTGGCGTCATCGAGCGCCA
TCTCGAACCGACGTTGCTGGCCGTACATTTGTACGGCTCCGCAGTGGATGGCGGCCTGAAGCCACACAGTGATAT
TGATTTGCTGGTTACGGTGACCGTAAGGCTTGATGAAACAACGCGGCGAGCTTTGATCAACGACCTTTTGGAAAC
TTCGGCTTCCCCTGGAGAGAGCGAGATTCTCCGCGCTGTAGAAGTCACCATTGTTGTGCACGACGACATCATTCC
GTGGCGTTATCCAGCTAAGCGCGAACTGCAATTTGGAGAATGGCAGCGCAATGACATTCTTGCAGGTATCTTCGA
GCCAGCCACGATCGACATTGATCTGGCTATCTTGCTGACAAAAGCAAGAGAACATAGCGTTGCCTTGGTAGGTCC
AGCGGCGGAGGAACTCTTTGATCCGGTTCCTGAACAGGATCTATTTGAGGCGCTAAATGAAACCTTAACGCTATG
GAACTCGCCGCCCGACTGGGCTGGCGATGAGCGAAATGTAGTGCTTACGTTGTCCCGCATTTGGTACAGCGCAGT
AACCGGCAAAATCGCGCCGAAGGATGTCGCTGCCGACTGGGCAATGGAGCGCCTGCCGGCCCAGTATCAGCCCGT
CATACTTGAAGCTAGACAGGCTTATCTTGGACAAGAAGAAGATCGCTTGGCCTCGCGCGCAGATCAGTTGGAAGA
ATTTGTCCACTACGTGAAAGGCGAGATCACCAAGGTAGTCGGCAAATAATAGGATCGTTTATTTACAACGGAATG
GTATACAAAGTCAACAGATCTCAACTCGAGACCTCAATGAATTCATTGGACCGCGGATCAAGGTACCAGGAGGTA
TTTATGGCTTTGCCTCAAACTGAACTACGACCTATGGGGAAACCCATATTAAGGAAAGAGGACCCACGATTAATC
CGAGGTAAGGGTCGTTTTGTTGATGATATATTATTACCAAATATGTTACACTTATGTATTTTAAGGTCCCCCTAT
GCTCACGCTAGGATACGACGTATCGATACCTCAAAAGCAGAGGCAGCTCCTGGCGTTAAATTAGTTCTTACTGGT
GAAGATTTAGCTAAAATGAATCTTGCCTGGATGCCCACTTTGGCTGGCGATGTCCAAATGGTCTTAGCCACAGGT
AAGGTACTTTTTCAATACCAAGAAGTTGCAGCAGTAGTTGCTGAAACTAGAGCGCAGGCAGAGGATGCTATTCAA
TTAATAGAAGTAGATTATGAACCTTTGCCTGTGGTAGTAGATCCCTTTAAAGCTCTTGAACCAGACGCTCCAATC
TTACGTGAAGATAAAGAAAAAAAATCAAATCATATCTGGCATTGGGAGGCCGGTGATAGAGAAGAAACAGATGCT
ATATTTCGAGAGGCCCCTGTGGTTGTAAAACAAGATGTACGATTTCAAAGAGTTCATCCCTCCCCACTTGAACCT
TGTGGATGTGTCGCTGATTACAATCCAGCTACTGGAAAACTTGTAGTATATGTTACGTCACAAGCGCCACATGTA
CATAGAACAGCAATTGCATTGACCACAGGATTTCCAGAACACATGATACAGGTTATTAGTCCGGATGTAGGGGGT
GGATTCGGAAATAAAGTTCCTCTTTATCCTGGTTATGTTGTGGCTATTGTAGCATCTTTAAAATTAGGTGTTCCT
GTTAAATGGATTGAGACCAGAACGGAAAATATTGCTTCTACACATTTTGCCAGAGACTATCACATGACCGCTGAA
ATTGCCGCTACGGAAGATGGTAAAATGTTAGCCCTTCGTGTTAAAACAATTGCTGATCATGGTGCCTTTGACGCT
ACAGCTAATCCTACCAAATATCCTGCTGGACTTTACTCTATAGTTACAGGAAGTTACGACTTTAAGGCAGCCTTT
GTTGAAGTAGATGGTGTACACACTAACAAACCTCCGGGAGGCGTAGCCTACCGATGCTCCTTTAGAGTTACAGAA
GCGAGTTATTTGATAGAACGAGTGGTTGATGTCTTGGCTAGACGATTAAAAATGGACCCCGCTGAATTAAGACTA
AGGAACTTCATTCGTAAGGAGCAATTTCCTTATAGAAGTCCCACTGGCTGGGTATACGATTCAGGTGATTATGAA
AAAACGTTCAAATTAGCTCTTGAGAGAATAGGGTATGAAGAACTACGTAAAGAGCAAAAAGAAAAATGGGCTAGA
GGAGAATTTATGGGTATCGGCATCAGTACTTTTACAGAAATTGTGGGAGCAGGACCAGCCCATTCATTCGATATA
TTAGGGATAAAAATGTTCGATTCAGCAGAAATCAGAGTGCATCCTACCGGAAAGGTTATTGCTCGTTTAGGTGTT
AGACATCAGGGCCAAGGTCATGAGACAACTTTTGCACAAATTATTGCAGAAGAACTTGGCCTTTCAGTTGATGAT
GTTGTAGTAGAGGAGGGTGATACGGATACAGCGCCTTATGGACTTGGAACCTATGCCTCTCGAAGTACACCAACT
GCCGGGGCAGCTGCGGCTTTGTGTGCTCGAAGAATTAGAGATAAAGCAAGAAAAATCGCAGCTCATCTTCTTGAG
GTAAACGAAGACGATGTAGTATGGGATGGCGCAGCTTTTTCTGTGAAAGGTTTACCAGGACGTTCTGTCACTATG
AAGGATGTAGCATTTGCTGCCTATACCAATGTGCCAGATGGCATCGAACCGGGTCTAGAGGCTAGTTATTATTAT
AATCCGCCAAACTTAACTTTTCCTTATGGTGCCTACATAGCAGTCGTTGACATTGATAAAGGAACTGGAGCGGTT
AAAGTACGAAGATTTTTAGCTGTAGATGATTGCGGAAATGTAATAAATCCGATGATAGTAGAAGGACAAGTCCAT
GGGGGTTTAACAGAAGGTTTTGCAATAGCGTTTATGCAAGATATACCTTATGATGCAGATGGGAACTGTCTAGCT
CCTAATTGGATGGATTACCTTGTACCAACGGCATGGGATACTCCGCAATTAGAGACAGATAGAACTGTGACCCCT
AGTCCTCATCATCCTTTGGGAGCAAAAGGAGTTGGAGAGTCTCCCAATGTCGGATCTCCCGCCGCATTCGTAAAT
GCTGTTCTAGATGCCCTATCTCCACTAGGTGTAGAACATATTGATATGCCTATTTATCCTTGGAAAGTCTGGAAA
ATATTACGAGACACCGCCCTTCGTTCTGATTCTATGGCTATTCCAGCTTCTTTCCAAAGTGCACGACGAGAGAAA
CCTGGCGGAGGTATTGCATCTGGACCCATTAAGTGGACTACATCTGGACGTCAACGAGGGAGATGGATGAATGCT
CGTTCTTTAACTTCTGGCTAATAGGATCGTTTATTTACAACGGAATGGTATACAAAGTCAACAGATCTCAAGCTA
GCAGGAGGTATTTATGAAAATTAGAGTAAAGGTTAACGGAACCTTATATGAAGCTGATGTTGAACCGCGTACCTT
ATTGGCTTATTTCTTACGTGAAGAACTTAAATTAACGGGCACTCATATTGGATGTGATACGACAACTTGCGGGGC
TTGTACTGTACTACTTGATGGAAAAGCGGTTAAATCTTGCACTGTACTAGCCGTACAAGCTAACGGCAGAGAGGT
TATGACAGTGGAAGGACTTGAAAAGGATGGTCAACTTCATCCTTTACAGGTTGCTTTTTGGGAGGAACATGCCCT
ACATTGTGGATACTGTACACCCGGTATGTTGATGGCTAGTTATGCTTTGTTACAGGAAAATCCGATGCCGACCGA
GGAAGAGATTAGATTCGGACTTTCAGGGAATGTTTGTCGATGTACTGGCTATATGAATATAGTCAAAGCTGTACA
ATCAGCAGCAAGACGTCTTAGTGGAGCTTCTGGTGAAGCTGTTGGAGAGGTAGCAACTTCTGGCACTGCTGCTGA
CTAATAGGATCGTTTATTTACAACGGAATGGTATACAAAGTCAACAGATCTCAAAGGAGGTATTTATGTTTCCCA
ATGCCTTCAAATATGAGGCTCCAGCTTCAGTAGATGAAGCAGTACGTCTATTAGCCGAGTATGGATATGATGGTA
AGGTTTTAGCTGGCGGTCAATCCTTGCTACCTATGATGAAACTACGAGTCGCTGCTCCTGCCGTACTTATTGATA
TAAATGGTATTGATGCGTTACAAGGATGGCGTGAAGTTGATGGGAAATTACGTGTCGGAGCCATGACACGTCATG
CGGAATTAGAACATGCAAAAGAGCTTAGGGATACTTATCCTTTGTTCTTCCAAACTGCGCGTTGGATTGCTGATC
CGTTAATCCGAAATAGAGGAACAATTGGAGGAAGTCTAGCTCATGCTGATCCAGGGTCTGACTGGGGGGCAGCAA
TGATTGCTTTACGAGCTGAGGTGGAAGCCCGTGGTCCTCAAGGGTCTCGTTTAATTCCCATTGACGAATTTTTTG
TTGATACTTTTGCCACCGCTTTAAATGAGGATGAATTGGCCGTTGCCGTACATGTACCGACACCTAAAGGGCCTG
CTGCATCACGATACATGAAACTAGAACGTCGAGCAGGTGATTTTGCTATAGCCGCTTTGGCAGTACATGTCGCAT
TAGGTACAGATGGTCGTGTCTCTGAAGCTGGTATTGGGATATGTGCTTGTGGTCCCATTCCGCTAAGAGCCGCCA
AAGCTGAAGCGGCTTTGATCGGACGTCCCTTAACTGAAGAAGTAATAGTAGAAGCGTCTAGATTGGTTCCAGAAG
ATGCTGAACCTGCCGATGACTTACGAGGTTCTGCCGAATATAAACGAGATGTACTTAGGGTATTCGCCGCCCGAG
CTTTAAGAGATATAGCAAAAGAACTTCAGGGCAAGGTTGGAATACAATAATAGGATCGTTTATTTACAACGGAAT
GGTATACAAAGTCAACAGATCTCAAAGGAGGTATTTATGTTTGAATTACCACCTTTACCATATCCGTACGACGCT
TTGGAACCGTATTTCGATGCAAAGACTATGGAAATTCATTATAATGGTCATCACGGTGCATACGTCAAGAATCTA
AATGCTGCTTTAGAAAAGTATCCTGCCTGGCAAAATAAGCCCATTGAAGAATTATTGCAATCTTTAGATCAGTTA
CCGGAAGATATTCGTACTGCTGTTCGAAATAACGGAGGCGGACATTATAACCATAGTTTTTGGTGGCCTATGTTG
AAAAAGAATGAGGGGGGTCAACCTGTAGGAAAATTTGCCGAAGCTATAAATCGTGATTTTGGTAGTTTTGAAGCG
TTTAAGGATGCTTTTTCCAAAGCCGCAGCTGGGCGTTTTGGATCTGGCTGGGCTTGGGTTGTAGTTGAGCCGGAT
GGAAAATTAACGGTCACCACAACTCCCAATCAAGATAATCCTGTTATGGAAGGGAAGACTGTAGTGTTTGGTTTG
GATGTTTGGGAACATGCTTATTATTTAAAATATCAAAATAGACGTCCGGAATACATACAGGCTTTTTGGAATGTC
GTAAATTGGGATGTAGTAAATGAACGATATGAAGAAGCTCTAAAAAAATTCGGCCGTTAATAGGATCGTTTATTT
ACAACGGAATGGTATACAAAGTCAACAGATCTCAACATATGACTGGAGGATCCACAAGGCCTATCAAGGCGCCAT
TAATTAAAGGCCGGCCAATTTAAATACAAGCTTGATCCTGGCCTAGTCTATAGGAGGTTTTGAAAAGAAAGGAGC
AATAATCATTTTCTTGTTCTATCAAGAGGGTGCTATTGCTCCTTTCTTTTTTTCTTTTTATTTATTTACTAGTAT
TTTACTTACATAGACTTTTTTGTTTACATTATAGAAAAAGAAGGAGAGGTTATTTTCTTGCATTTATTCATGATT
GAGTATTCTATTTTGATTTTGTATTTGTTTAAAATTGTAGAAATAGAACTTGTTTCTCTTCTTGCTAATGTTACT
ATATCTTTTTGATTTTTTTTTTCCAAAAAAAAAATCAAATTTTGACTTCTTCTTATCTCTTATCTTTGAATATCT
CTTATCTTTGAAATAATAATATCATTGAAATAAGAAAGAAGAGCTATATTCGACCTGCAGACTACTTCATGCATG
CTCCACTTGGCTCGGGGGGATATAGCTCAGTTGGTAGAGCTCCGCTCTTGCAATTGGGTCGTTGCGATTACGGGT
TGGATGTCTAATTGTCCAGGCGGTAATGATAGTATCTTGTACCTGAACCGGTGGCTCACTTTTTCTAAGTAATGG
GGAAGAGGACCGAAACGTGCCACTGAAAGACTCTACTGAGACAAAGATGGGCTGTCAAGAACGTAGAGGAGGTAG
GATGGGCAGTTGGTCAGATCTAGTATGGATCGTACATGGACGGTAGTTGGAGTCGGCGGCTCTCCCAGGGTTCCC
TCATCTGAGATCTCTGGGGAAGAGGATCAAGTTGGCCCTTGCGAACAGCTTGATGCACTATCTCCCTTCAACCCT
TTGAGCGAAATGCGGCAAAAGAAAAGGAAGGAAAATCCATGGACCGACCCCATCATCTCCACCCCGTAGGAACTA
CGAGATCACCCCAAGGACGCCTTCGGCATCCAGGGGTCACGGACCGACCATAGAACCCTGTTCAATAAGTGGAAC
GCATTAGCTGTCCGCTCTCAGGTTGGGCAGTCAGGGTCGGAGAAGGGCAATGACTCATTCTTAGTTAGAATGGGA
TTCCAACTCAGCACCTTTTGAGTGAGATTTTGAGAAGAGTTGCTCTTTGGAGAGCACAGTACGATGAAAGTTGTA
AGCTGTGTTCGGGGGGGAGTTATTGTCTATCGTTGGCCTCTATGGTAGAATCAGTCGGGGGACCTGAGAGGCGGT
GGTTTACCCTGCGGCGGATGTCAGCGGTTCGAGTCCGCTTATCTCCAACTCGTGAACTTAGCCGATACAAAGCTT
TATGATAGCACCCAATTTTTCCGATTCGGCGGTTCGATCTATGATTTATCATTCATGGACGTTGATAAGATCCAT
CCATTTAGCAGCACCTTAGGATGGCATAGCCTTAAAAGTGAAGGGCGAGGTTCAAACGAGGAAAGGCTTACGGTG
GATACCTAGGCACCCAGAGACGAGGAAGGGCGTAGTAATCGACGAAATGCTTCGGGGAGTTGAAAATAAGCATAG
ATCCGGAGATTCCCGAATAGGGCAACCTTTCGAACTGCTGCTGAATCCATGGGCAGGCAAGAGACAACCTGGCGA
ACTGAAACATCTTAGTAGCCAGAGGAAAAGAAAGCAAAAGCGATTCCCGTAGTAGCGGCGAGCGAAATGGGAGCA
GCCTAAACCGTGAAAACGGGGTTGTGGGAGAGCAATACAAGCGTCGTGCTGCTAGGCGAAGCAGCCCGAATGCTG
CACCCTAGATGGCGAAAGTCCAGTAGCCGAAAGCATCACTAGCTTATGCTCTGACCCGAGTAGCATGGGGCACGT
GGAATCCCGTGTGAATCAGCAAGGACCACCTTGCAAGGCTAAATACTCCTGGGTGACCGATAGCGAAGTAGTACC
GTGAGGGAAGGGTGAAAAGAACCCCCATCGGGGAGTGAAATAGAACATGAAACCGTAAGCTCCCAAGCAGTGGGA
GGAGCCAGGGCTCTGACCGCGTGCCTGTTGAAGAATGAGCCGGCGACTCATAGGCAGTGGCTTGGTTAAGGGAAC
CCACCGGAGCCGTAGCGAAAGCGAGTCTTCATAGGGCGGCCGCCCGGGTAATACGGTTATCCACAGAATCAGGGG
ATAACGCAGGAAAGAACATGTGAGCAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGCCTTATCCGGTA
ACTATCGTCTTGAGTCCAACCCGGTAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATGATACCGC
GAGACCCACGCTCACCGGCTCCAGATTTATCAGCAATAAACCAGCCAGCCGGAAGGGCCGAGCGCAGAAGTGGTC
CTGCAACTTTATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTTCATTCAGCT
CCGGTTCCCAACGATCAAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTGCATAATTCTCTTACTG
TCATGCCATCCGTAAGATGCTTTTCTGTGACTGGTGAGTACTCAACCAAGTCATTCTGAGAATAGTGTATGCGGC
GACCGAGTTGCTCTTGCCCGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACAGGAAGGCAAAATG
CCGCAAAAAAGGGAATAAGGGCGACACGGAAATGTTGAATACTCATACTCTTCCTTTTTCAATATTATTGAAGCA
TTTATCAGGGTTATTGTCTCATGAGCGGATACATATTTGAATGTATTTAGAAAAATAAACAAATAGGGGTTCCGC
GCACATTTCCCCGAAAAGTGCCACCTGACGTCTAAGAAACCATTATTATCATGAC
SEQ ID NO: 28, Primer P1
CAAAATAGAATACTCAATCATG
SEQ ID NO: 29, Primer P2
AGATATAGCAAAAGAACTTC
SEQ ID NO: 30, Primer P3
CGTGGTGATTGATGAAACTG

1-15. (canceled)

16. A plant cell, which is capable to fix atmospheric nitrogen, wherein said cell expresses nucleotide sequences encoding for Streptomyces thermoautotrophicus nitrogenase.

17. The plant cell of claim 16, wherein said Streptomyces thermoautotrophicus nitrogenase subunits' amino acid sequences comprise the amino acid sequences shown in SEQ ID NOs: 21-24.

18. The plant cell of claim 16, wherein one or more of said Streptomyces thermoautotrophicus nitrogenase amino acid subunit sequences has at least 80% sequence identity or similarity to the amino acid sequences shown in SEQ ID NOs: 21-24, and wherein said Streptomyces thermoautotrophicus nitrogenase exhibits from about 10% to about 200%, or more, of the nitrogen fixing activity of Streptomyces thermoautotrophicus nitrogenase comprising SEQ ID NOs: 21-24.

19. The plant cell of claim 17, wherein one or more of said Streptomyces thermoautotrophicus nitrogenase amino acid subunit sequences has at least 80% sequence identity or similarity to the amino acid sequences shown in SEQ ID NOs: 21-24, and wherein said Streptomyces thermoautotrophicus nitrogenase exhibits from about 10% to about 200%, or more, of the nitrogen fixing activity of Streptomyces thermoautotrophicus nitrogenase comprising SEQ ID NOs: 21-24.

20. A plant cell capable to fix atmospheric nitrogen, wherein said cell expresses Streptomyces thermoautotrophicus nitrogenase subunits from nuclear, plastid, or mitochondrial genomes, or as an episome, or in combinations of any of the foregoing.

21. The plant cell of claim 20, wherein one or more of said Streptomyces thermoautotrophicus nitrogenase subunits further comprises a plastid targeting sequence.

22. A plant cell of claim 20, wherein Streptomyces thermoautotrophicus nitrogenase subunits are optimized for expression or activity in plant cellular or organellar environment.

23. Progeny, derivatives or parts of plant or algae capable of nitrogen fixation and expressing Streptomyces thermoautotrophicus nitrogenase.

24. The progeny, derivatives or parts of claim 23, selected among clones, hybrids, samples, seeds, cells and harvested material thereof.

25. The progeny or derivatives of claim 23, produced sexually or asexually.

26. The plant part of claim 23, which is selected from a protoplast, a cell, a tissue, an organ, a cutting, an explant, a reproductive tissue, a vegetative tissue, an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an auxillary bud, a leaf, a stem, a root, a tuberous root, a rhizome, a tuber, a stolon, a corm, a bulb, an offset, a cell of said plant in culture, a tissue of said plant in culture, an organ of said plant in culture, a callus, a homogenate, propagation material, germplasm, cuttings, divisions and propagations.

27. A plant product obtained from the plant progeny, derivatives or parts of claim 23.
